(19) United States
(12) Patent Application Publication
(10) Pub. No.: US 2023/0001128 A1
(43) Pub. Date: Jan. 5, 2023

(54) AEROSOLIZATION SYSTEMS, METHODS, AND APPARATUSES

(71) Applicants: Gary Stephen SHUSTER, Vancouver (CA); Brian SHUSTER, Vancouver (CA)

(72) Inventors: Gary Stephen SHUSTER, Vancouver (CA); Brian SHUSTER, Vancouver (CA)

(21) Appl. No.: 17/779,542

(22) PCT Filed: Nov. 24, 2020
(86) PCT No.: PCT/US20/62096

§ 371 Date: May 24, 2022

Related U.S. Application Data
(60) Provisional application No. 62/940,101, filed on Nov. 25, 2019.

(51) Int. Cl. A61M 21/02; A61M 11/04; A24F 40/48; A24F 40/20; A47J 27/00

(52) U.S. Cl. CPC A61M 21/02; A61M 11/042; A24F 40/48; A24F 40/20; A47J 27/004; A61M 2202/0208; A61M 2202/0291; A61M 2205/7545

(57) ABSTRACT

Systems, methods and apparatuses for aerosolizing all or substantially all plant matter, medications, flavors, smells, liquid and/or other material to be aerosolizing are disclosed. Embodiments of the invention comprise an aerosolization chamber sealed except for two or more conduits, a first conduit coupled to a source of fully or almost fully oxygenated gas, a heating element capable of heating the aerosolization chamber to a temperature above a combustion temperature, a second conduit configured to transport aerosolized gases and elements out of the chamber and, in one implementation, at least one valve positioned in the second conduit preventing the flow of atmospheric air into the vaporization chamber. In some instances, the first gas substantially clears the vaporization chamber of atmospheric air prior to reaching combustion temperature. A second gas containing oxygen may be intermixed with the vaporization gases and vaporized elements proximal to the combustion chamber.

### CROSS-REFERENCE TO RELATED APPLICATION

Pursuant to 35 U.S.C. § 371, this application is a U.S. National Phase application of PCT/US2020/062096 filed Nov. 24, 2020, which claims priority pursuant to 35 U.S.C. § 119(e) to U.S. provisional patent application No. 62/940,101, filed Nov. 25, 2019, which applications are specifically incorporated herein, in their entireties, by reference.

### FIELD OF INVENTION

The present disclosure relates to vaporization and/or aerosolization of plant matter, pharmaceuticals, or other materials.

### DESCRIPTION OF RELATED ART

Vaporization, the conversion of a substance from the liquid or solid phase into a gaseous or vapor phase, has seen growing adoption as a way to inhale physiologically and/or psychologically active compounds without inhaling byproducts of combustion. Even biologically inactive substances may be vaporized for the flavor or experience. For example, tobacco leaves heated to 140-200° C. will aerosolize certain of the organic compounds in the tobacco leaves, allowing inhalation without also inhaling combustion byproducts. It is not unusual to heat plant matter to, or even slightly past, the boiling point of biological materials, such as nicotine, Cannabidiol (“CBD”), or Tetrahydrocannabinol (“THC”), yet some of the material does not vaporize even at that boiling point. This may be due to a variety of factors, such as the material being embedded within plant matter in a way that traps the substance. Uneven heating is another problem with vaporizers, in that a device set to 220 degrees may have hot spots at 240 degrees or higher and cool spots too low to fully boil off desirable materials. A common complaint among users of plant matter vaporizers is that they find materials that have been partially vaporized in this way still contain valuable molecules that have not been vaporized. An additional problem with this mechanism is that uneven heating may result in combustion, with the risk of combustion increasing with increased air flow.

Vaporizers are also used to release scents from plants, flavors and scents from spices, and for other purposes. One growing use of vaporizers is for the consumption of medical cannabis. Certain studies, such as a 2007 study by University of California, San Francisco and published in the Journal of the American Academy of Neurology, have found substantial safety benefits from the use of a vaporizer when compared to combustion. As that study states, “there was virtually no exposure to harmful combustion products using the vaporizing device.”

However, there are several problems with vaporizing. A primary problem is that plant matter heats unevenly in a vaporizer. Unlike a combustion device, a vaporizer may leave the structure of the plant matter intact (whereas combustion turns it to ash). As a result, it is important to grind the plant matter into small pieces in order to minimize the amount of organic matter trapped inside of plant structure and to allow more even heating. However, there is a maximum amount of grinding that is possible before the matter becomes small enough to pass through any filters or grates. Put another way, vaporizing will always leave some amount of organic matter non-aerosolized. Looking at the problem in different terms, surface area is a crucial factor in changing vaporizer efficacy. Using cannabis as an example, one may put cannabis into a coffee grinder and reduce it to a fine powder, maximizing surface area. However, the increase in surface area increases the likelihood of combustion in the event of uneven heating while at the same time increases the amount of plant matter that is aerosolized as a simple function of a small size (a small enough size, in many cases, to pass through filtering mechanisms). As a practical matter, loss of source material may occur with greater frequency as the particle size reduces, making the material more susceptible to being lost to simple air flow.

Another problem with vaporizers is that the heat source is frequently inconsistent. For example, some vaporizers use resistance heating, creating hot spots near the wires. Others use passive-convection, with differing amounts of heat passing through differing parts of the apparatus. One of the side effects of vaporizing is that liquid present in the plant matter evaporates, changing the susceptibility of the remaining plant matter to combustion. It is thus desirable to change vaporizing temperature as the matter dries out in order to avoid combustion of the matter—but at the same time, matter already dried out by heat is unlikely to efficiently boil off additional psychoactive material as the material subject to boiling at temperature X is likely to largely have already boiled off before being subjected to temperature (X minus Y). Regardless, it is common for vaporizing at higher temperatures to result in some amount of combustion. Further, while combustion is a rapid means of oxidation, it should be appreciated that certain organic compounds combine with oxygen at lower temperatures.

Approaches described in this section are approaches that could be pursued but are not necessarily approaches that have been previously conceived or pursued. Therefore, no admission is made, nor should it be assumed, that any of the approaches described in this section qualify as prior art merely by virtue of their inclusion.

### SUMMARY OF INVENTION

Combustion cannot take place when the oxygen content of the gas is below the limiting oxygen concentration (“LOC”). The LOC for paper, for example, in a Nitrogen/air mixture at average sea level air pressure (1013.25 mbar), is approximately 14.1%. However, it is important to understand that combustion is in essence a self-sustaining exothermic process. Heat is initially applied to material to initiate combustion, after which the combustion itself becomes the heat source to continue combustion so long as the oxygen remains above the LOC and fuel remains to burn. Simply avoiding combustion does not avoid all oxidation. Fire is the rapid oxidation of fuel in the exothermic combustion process. However, slower reduction-oxidation (or simply oxidation) is possible even below the LOC. A common example is rust. Any vaporizer device not utilizing the inventions described herein will release some amount of oxidation byproducts. Indeed, some oxidation may happen even at room temperature. It is desirable to avoid oxidation entirely in any situation where a living being breathes gas that includes oxidation byproducts. Byproducts of oxidation may include, for example, carbon dioxide and carbon monoxide. Using reaction gases other than oxygen may produce some reduction or other chemical interaction.

Embodiments of the invention utilize an atmosphere below the LOC to allow for the safe vaporization of a variety of materials. Embodiments further provide for a mechanism by which oils with a low smoke point may be utilized at high temperatures.

It is to be understood that both the foregoing general description and the following detailed description are exemplary, but not restrictive, of the invention. A more complete understanding of the improved vaporizer and the methods disclosed herein will be afforded to those skilled in the art.

### BRIEF DESCRIPTION OF THE DRAWINGS

FIG. 1 is a diagram of a device for vaporizing Vaporization Targets.

FIG. 2 is a diagram of a device for vaporizing Vaporization Targets, showing a sealant to prevent entry of atmospheric oxygen.

FIG. 3 is a diagram of a cartridge with an integral heating element.

FIG. 4 is a diagram of a device for vaporizing Vaporization Targets with two heating chambers.

FIG. 5 is a diagram of an extraction chamber.

FIG. 6 is a diagram of a cooking device.

FIG. 7 is a diagram of a self-contained medication vaporizer.

FIG. 8 is a diagram of a low pressure vaporizer.

### DETAILED DESCRIPTION OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications, and equivalents that may be included within the spirit and scope of the invention. Furthermore, in the following detailed description of the present invention, numerous specific details are set forth in order to provide a thorough understanding of the present invention. However, it will be readily apparent to one skilled in the art that the present invention may be practiced without these specific details. In other instances, well-known methods, procedures and components have not been described in detail so as not to unnecessarily obscure aspects of the present invention. These conventions are intended to make this document more easily understood by those practicing or improving on the inventions, and it should be appreciated that the level of detail provided should not be interpreted as an indication as to whether such instances, methods, procedures or components are known in the art, novel, or obvious.

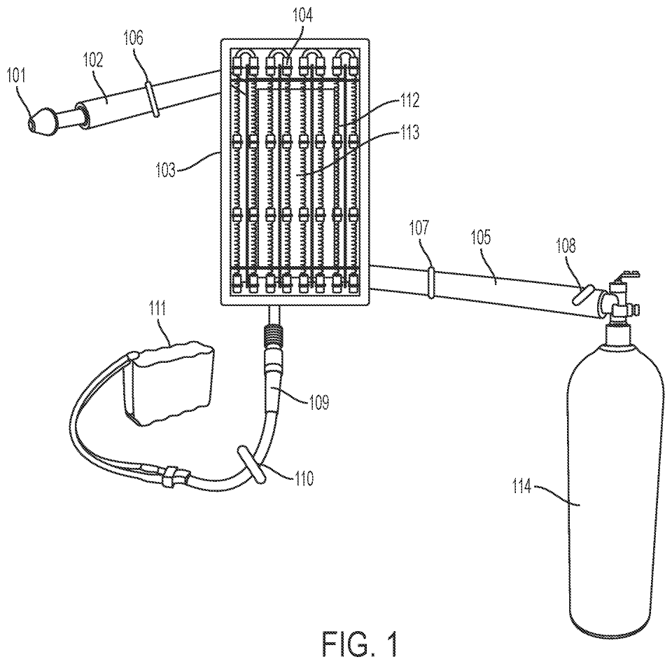

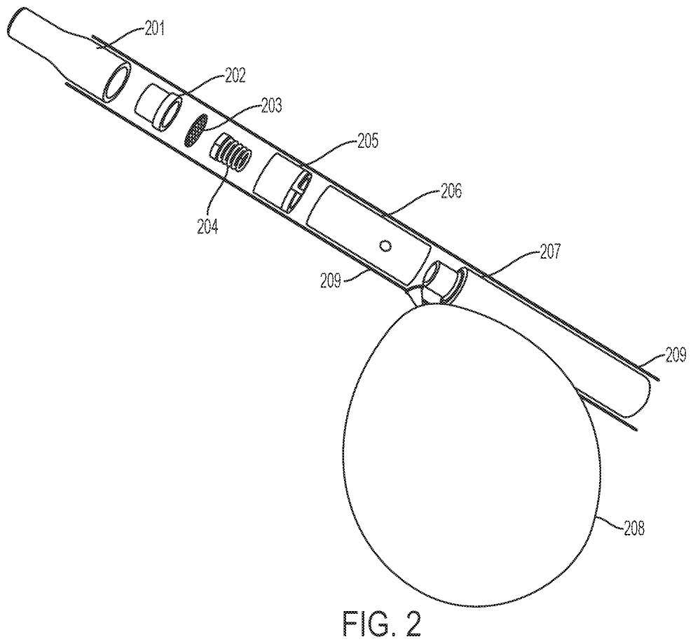

In understanding the instant inventions, it is useful to review CA3009402C, and pending US national phase counterpart US20190174833A1 (“Vaporizer Improvements”). In Vaporizer Improvements, the instant inventors teach, among other things, utilizing a non-oxygenated atmosphere as the gas within which plant matter, including cannabis or tobacco, may be heated to above the temperature at which combustion would normally take place. FIGS. 1 and 2 of the instant application are duplicates of FIGS. 1 and 2 of Vaporizer Improvements and are used in this document to provide context to the new inventions.

Turning to FIG. 1, we note the following elements:

101: A mouthpiece suitable for breathing through.

102: Hollow tube, which acts as a conduit between the heating chamber and the mouthpiece 101 to carry the vaporized material and the gas from the heating chamber to the consumer.

103: Vaporization chamber.

104: Heating element. If resistance heating, may utilize resistors such as Nichrome or KANTHAL®. It may also utilize a Peltier heating device, in which case cooling elements, such as those described in 106, may (but need not) be the cool side of the Peltier heating device.

105: Hollow tube, through which non-oxygenated gas or a gas mixture is delivered from gas source 114 to the vaporization chamber 103.

106: Connector between Hollow Tube 102 and Vaporization chamber 103. Connector may contain cooling elements or a valve preventing passage of gas exceeding a set temperature (although one or both of those elements may be placed at other points between the vaporization chamber 103 and the mouthpiece 101, or even integral with the mouthpiece).

107: Connector between hollow tube 105 and vaporization chamber 103. In one aspect, the connector (or valve 108 or other element between the gas container 114 and the vaporization chamber 103) may detect oxygen and warn or prevent the flow of gas.

108: Valve. Valve may be mechanical, electro-mechanical, or otherwise. In one aspect, Valve may be actuated by negative air pressure in the tube 105. In one aspect, the valve may be held closed with a spring.

109: Wiring connection (or wireless connection such as inductive charging) for power.

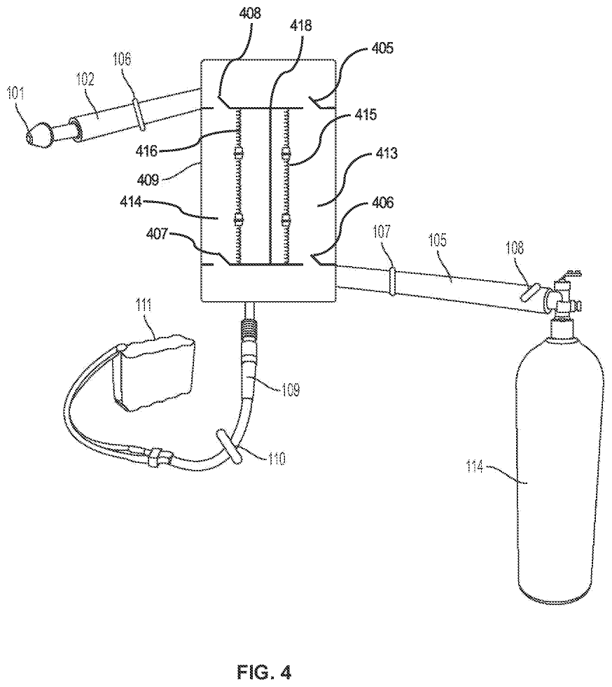

110: Switch (physical switch, digital switch, electro-mechanical or otherwise) to control power. May be controlled by microprocessor, thermistor or other temperature-sensitive switching system. May control amount of current to achieve target temperature in chamber 103 (or chamber 403, see FIG. 4 and related description beginning at para. [0088]).

111: Power source (direct and/or inductive).

112: (optional) pre-packed or packable container for holding plant matter (or other material for vaporization).

113: An airspace within the vaporization chamber 103 for plant matter (or other material for vaporization).

114: A gas source for providing either non-oxygenated gas (or set amount of oxygen, or minimal amount of oxygen), gas below the LOC or a gas mixture. In one aspect, the non-oxygenated gas may be helium. In another aspect, the gas source may be a balloon or a tank.

FIG. 2 utilizes the vaporizer structure described in the related art section as a starting point. Distinguishing from this structure are several elements, shown and not shown. Unlike existing vaporizers, a sealant 209 is utilized to prevent oxygenated gas from entering. A gas input 208 delivers a non-oxygenated (or minimally oxygenated) gas. The gas input may contain a valve. In one aspect, the valve may be actuated by negative air pressure in the chamber 206. The gas source 208 may be a balloon, a canister of gas, or another source.

Element 201 is the mouthpiece.

Element 202 is a first filter to prevent plant matter from entering the lungs.

Element 203 is a mesh filter to prevent plant matter from entering the lungs.

Element 204 is a spring.

Element 205 is a connector to couple the chamber to the filter and mouthpiece assembly. Within this element (or at any point between or including the heating chamber and the mouthpiece), a cooling element (such as water or a Peltier cooling element) and/or a valve that closes if the gas temperature is unsafe or too high, may be present.

Element 206 is the heating or vaporization chamber. In one aspect, it may be ceramic.

Element 207 is a power source.

Element 208 is a non-oxygenated gas source. A valve may be present at the connection of this element to the remainder of the system (or elsewhere between the source and the mouthpiece).

Element 209 is a sealant or sealing matter to prevent entry of atmospheric oxygen.

As Vaporization Improvements teaches, the devices in FIGS. 1 and 2 may be utilized to vaporize plant matter without combustion byproducts.

The instant inventions teach a variety of novel technologies.

When substituting an anoxic gas for atmospheric air, there are a variety of choices. In many cases, it is desirable to utilize a noble gas atmosphere, such as pure (or a mixture containing) Helium, Neon, Argon, Krypton, or Xenon to avoid (for pure gases) or minimize (for mixtures) oxidation and reduction. Noble gases with a heavier atomic weight, such as Radon, are subject to radioactive decay in a manner that largely renders them undesirable for inhalation, though they may be used despite the risk. Only a few atoms of Oganesson have ever been synthesized, and with an atomic number of 118, it is quite unstable (unlike the lighter noble gasses) and may be highly reactive (unlike the other zero-valence elements). In other cases, it is desirable to use inert or functionally or substantially inert gas molecules, such as nitrogen (N₂) (N₂ is referred to herein as “Nitrogen”).

A brief discussion of the use of nitrous oxide (N₂O) in the inventions is desirable at this point. N₂O is not stable at high temperatures and may result in the release of oxygen. Free oxygen within the heating chamber may result in combustion. Furthermore, while N₂O could be added to the gas after heating and aerosolization takes place, we respectfully teach that such a combination should only be attempted under the supervision of, and upon the orders of, a physician. We teach herein that N₂O may be utilized in combination with this invention; however, the preferred method of utilizing it is to add it to the stream of post-heated gas in an amount medically approved.

Xenon is known to have sedating properties. In one aspect, it is desirable to utilize a sedating product, such as Indica strains of cannabis (though all cannabis has sedating properties, so this is not limited to any particular strain or brand of cannabis, and may be used with other materials with sedating properties, such as benzodiazepines), in combination with a Xenon or partial Xenon atmosphere. In this way, the user is immediately partially sedated by the Xenon which, in combination with pharmaceuticals heated as described herein and/or plant matter heated as described herein, provides a sedating effect that is quite suitable for those with sleep related issues such as insomnia. In one aspect, Xenon is one of the gases in which the matter is heated. In one implementation, an amount of Xenon is used in the heating chamber and/or added to the output from the heating chamber, and/or in a final combination equal to a designated percentage depending on the desired sedative effect, which may be anywhere in the range of 0.1% to 100% (e.g., 0.1%, 0.2%, 0.7%, 1.2%, 1.3%, 1.9%, 2.0%, 2.1%, 2.4%, 2.5%, 3.3%, 3.6%, 4.5%, 4.6%, 5.0%, 5.9, 6.3%, 7.8%, 8.1%, 8.9%, 9.0%, 9.1%, 10.0%, 10.7%, 12.5%, 14.3%, 17.9%, 20.2%, 25.0%, 33.3%, 40.4%, 40.5%, 48.4%, 51.3%, 62.5%, 71.0%, 72.5%, 73.9%, 75.0%, 83.2%, 86.8%, 89.7%, 91.9%, 95.3%, 95.7%, 95.8%, 98.4%, 99.9%, 100%), or any percentage or range in between.

In one aspect, the system may be utilized to allow the boiling off of cannabinoids or other substances that are normally not made bioavailable because the boiling temperature exceeds the combustion temperature of cannabis. Cannabis has compounds that combust below 233 degrees Celsius (or Fahrenheit 451), a temperature well known as the combustion temperature of paper. THCV boils at 220 degrees C., meaning that much of it will be lost to combustion. CBDA boils between 316 and 531 degrees C.; Pulegone boils at 224 degrees C.; a-terpineol boils at 218 degrees C.; terpineol-4-ol boils at 209 degrees C.; quercetin boils at 250 degrees C.; there are other cannabis compounds that boil at a variety of temperatures, some well above combustion. Just as THCA must be decarboxylated in order to have a full psychoactive effect in humans, so too are there other compounds in cannabis that are believed to be altered at high temperatures to generate a greater psychoactive effect. By heating in a non-oxygenated atmosphere, these compounds can be released and/or altered in order to increase the amount of psychoactive (or other medically active) compounds released from the same amount of plant matter.

For reference, we provide a summary of a subset of cannabis-borne compounds in Table 1:

| | | Boiling Point |
|---|---|---|
| Compound name | Effect | in degrees C. |
| Δ9-Tetrahydrocannabinol (Δ9-THC) | Psychoactive | 155 to 160 |
| Δ8-Tetrahydrocannabinol (Δ8-THC) | Psychoactive | 175 to 178 |
| Tetrahydrocannabivarin (THCV) | Analgesic, euphoriant | Under 220 |
| Tetrahydrocannabinolic Acid | Δ9-THC precursor | Undetermined |
| (THCA) | | |
| Tetrahydrocannabivarin | Non-psychoactive | Undetermined |
| Carboxylic Acid (THCVA) | | |
| Cannabidiol (CBD) | Minimally or non- | 160 to 180 |
| | psychoactive; anti-convulsant; | |
| | anti-inflammatory; other | |
| | medicinal properties; can be | |
| | converted to Δ9-THC and Δ8- | |
| | THC with acidic treatment | |
| | such as cyclohexane. | |
| Cannabidiolic Acid | Minimally or non- | Undetermined |
| (CBDA) | psychoactive; generated from | |
| | cannabigerol acid (CBGA) | |
| | and is the carboxylated | |
| | precursor of cannabidiol | |
| | (CBD). At its boiling point, | |
| | CBDA begins to | |
| | decarboxylate into CBD. | |
| Cannabidivarin (CBDV) | Minimally or non- | Undetermined |
| | psychoactive; low oral | |
| | bioavailability (~6%). | |
| Cannabidivarin Acid | Minimally or non- | Undetermined |
| (CBDVA) | psychoactive; acidic form of | |
| | CBDV | |
| Cannabichromenic Acid | Minimally or non- | 435 to 525 |
| (CBCA) | psychoactive; decorboxildates | |
| | into CBC | |
| Cannabichromene (CBC) | Minimally or non- | 220 |
| | psychoactive; CBC may be a | |
| | by-product from the synthesis | |
| | of CBCA and Δ9-THC from | |
| | CBG; may be converted to | |
| | CBL. | |
| Cannabigerol (CBG) | Minimally or non- | Melts at 52 |
| | psychoactive; CBG can be | |
| | converted to CBD, THC, and | |
| | CBN. | |
| Cannabicyclol (CBL) | Minimally or non- | Undetermined |
| | psychoactive | |
| Cannabinol (CBN) | Minor psychoactivity; a | 185 |
| | crystalline alcohol that is a | |
| | minor by-product of Δ9-THC | |
| | oxidation | |
| Cannabinolic Acid | Minimally or non- | 478 to 579 |
| (CBNA) | psychoactive; acidic precursor | |
| | to CBN; a metabolite of | |
| | THCA oxidation; may be | |
| | synthesized from THCA. | |
| α-Pinene | Thought to function as a | 155 to 156 |
| | bronchodilator, thought to | |
| | have anti-inflammatory, | |
| | stimulant, antibiotic, | |
| | antineoplastic properties; | |
| | possible AChE inhibitor | |
| Limonene | Research into anti-cancer and | 176-177 |
| | anti-depressive properties | |
| | underway. | |
| Myrcene | May boost THC effects | 168 |
| Beta-caryophyllene | Research into anti-anxiety, | 119 to 160 |
| | anti-inflammatory, and tumor | |
| | reduction underway. | |
| Linalool | Thought to aid sleep, reduce | 198 |
| | psychosis, epilepsy and | |
| | anxiety. | |
| β-myrcene | Thought to have analgesic, | 166 to 168 |
| | anti-inflammatory, antibiotic, | |
| | and antimutagenic properties | |
| 1,8-cineole (eucalyptol) | Thought to increase cerebral | 176 |
| | blood flow, may inhibit AChE, | |
| | may have stimulant; antibiotic, | |
| | antiviral, anti-inflammatory, | |
| | antinociceptive properties. | |

As can be seen from the chart above, research into bioavailable substances from cannabis tends to end at or about the combustion point, presumably because the substances with higher boiling points are destroyed by combustion prior to becoming bioavailable. With the instant inventions, such substances may be released, synthesized, used and/or studied.

Yet another problem with both combustion and vaporization technology is that the odor of heated plant matter is often quite noticeable. By preventing the plant from oxidizing, the smell generated is quite different than that most people associate with the plant matter burning or being vaporized.

It should be noted that one approach to optimal vaporization/aerosolization is to use a lower initial temperature, optionally clear the heating chamber before the lower temperature reaches an upper limit, and repeat the process. In this way, aerosolized materials are not subjected to higher heat than necessary, reducing anaerobic pyrolysis or heat-related reactions. In another aspect, a vibratory motor or other movement or kinetic energy source may be utilized to move the particles of matter being heated, improving efficiency.

With regard specifically to vaporization of cannabis, it should be understood that Tetrahydrocannabinolic acid (THCA) is a biosynthetic precursor to the psychoactive THC. THCA has no known psychoactive effects on humans, although there is research showing significant medical impacts of THCA, such as anti-inflammatory and antiemetic properties. Heating THCA causes substantial conversion to THC, whereas ingestion of THCA leads to a highly limited amount of in vivo conversion to THC. A failure to sufficiently heat the THCA may lead to incomplete conversion of the totality of THCA within the plant matter. Further, one of the reasons why cannabis needs to be dried prior to use in prior art systems such as a bong or joint is to further ease the conversion of THCA to THC.

Fire is an exothermic chemical process of combustion leading to rapid oxidation of a material. A popular additive to e-cigarettes is propylene glycol/glycerin. Propylene glycol may degrade in the presence of oxygen. Any combustion process will lead to undesirable chemical byproducts. Indeed, even a hypothetical fully efficient combustion process will result in at least the creation of carbon dioxide, a substance that is undesirable for inhalation. It should be noted that oxidation and combustion refer to largely the same end result—a chemical reaction between a substance and oxygen. While we use the terms oxidation and combustion by themselves herein, unless the context clearly requires, each use of one term should be understood to refer to oxidation, by itself, combustion, by itself, and/or the combination of the two.

Returning to the problem of uneven distribution of heat in vaporization, a workaround that is only partially effective is to break the plant matter into very small pieces. Similarly, with liquid vaporization, thermally conductive materials may be used. However, even a slightly uneven heat distribution may lead to some areas of material being vaporized, others not vaporized, and yet other areas being burned or combusted. In addition, substances prepared for vaporization in a non-naturally occurring form, such as in an oil suspension, are frequently extracted utilizing undesirable chemicals, and may additionally contain substances that are undesirable to inhale. As carbon dioxide, carbon monoxide and other undesirable and/or harmful combustion byproducts combine atmospheric oxygen with product intended for vaporization, there is an upper temperature limit for vaporization in order to avoid combustion. However, at this upper limit, there is still insufficiently even heating to efficiently vaporize all of the material.

Existing vaporization technology is insufficient to address these issues. One exemplary existing vaporizer is the Bronze Sherlock VG Vaporizer (http://vaporgenie.com/bronze-sherlock-vaporizer). It is described as working as follows: “The VaporGenie pipe has a ceramic filter in the sphere above the bowl. The ceramic filter is designed to thoroughly mix heat from flame with cold, ambient air, producing an air stream with a temperature of about 275-350° F. [135-176.7° C.]. This air stream is hot enough to vaporize plant essences, but not hot enough to burn.” As noted above, this temperature will only vaporize a portion of the desired “plant essences”.

Other vaporizers use internal heating elements; examples may be found at http://www.mrniceguyheadshops.com/blog/2014/12/22/vaping-what-is-it-best-vaporizers.

Additionally, a popular vaporizer is the “Volcano”, which may be found at http://www.volcanovape.net/. It should be noted that U.S. Pat. No. 6,513,524B1 (expired) appears to describe the Volcano.

The Volcano vaporizer utilizes a system that pumps in atmospheric air, which is heated to temperatures needed for vaporization. Hot air passes over aromatic blends or essential oils to create vapor, filling the Volcano balloon by method of forced air. Once the balloon is filled with vapor, it is ready to be detached and used (see also: http://www.vapeworld.com/volcano-vaporizer-blazing).

It should be understood that for clarity, it was necessary to describe previously existing art (such as the Volcano) to illustrate improvements in the existing inventions. As a result, approaches described in this section and in the related art section are approaches that could be pursued but are not necessarily approaches that have been previously conceived or pursued. Therefore, no admission is made, nor should it be assumed, that any of the approaches described in this section or the related art section qualify as prior art merely by virtue of their inclusion in either section.

It is desirable to provide a system, method, and apparatus for vaporizing all or substantially all plant matter and/or liquid and/or other material to be vaporized (this grouping of all vaporization targets is referenced herein as “Vaporization Targets” and include each of the foregoing unless the context requires otherwise). It is further desirable that such system, method or apparatus avoid oxidation and/or reduction to the extent possible.

For ease of discussion, the portion of the device between the vaporization chamber and the mouthpiece is sometimes referenced as the “proximal” portion of the device, while the portion of the device between the vaporization chamber and the non-oxygenated gas source is sometimes referenced as the “distal” portion of the device.

The instant inventions utilize one or more of, among other things, a heating element, a heating chamber, a power source, a source of gas other than atmospheric gas or other gas containing any (or more than a threshold amount of) oxygen or O2 (for example, helium, argon, or nitrogen), a cooling mechanism (which may simply be piping, an intermix with atmospheric air, O2, or an O2 mixture, or may be more complex) and a delivery system (whether direct delivery, such as a mouthpiece, or delayed delivery, such as storing the output in a balloon, bag or sealed container).

In one aspect, it should be noted that it is desirable to utilize a gas (or combination of gasses) that is lighter than air, as there is a risk of asphyxiation if one inhales a gas that is significantly heavier than air. There are potential radiation risks to utilizing Radon. Argon, which is heavier than air, is not sufficiently heavier than air to pose such a risk.

Xenon, or Xe, has an atomic number of 54 and is generally non-reactive. Although the precise mechanism of action of Xenon is unclear, Xenon functions as a general anesthetic when used on certain animals, including humans.

In one aspect, Xenon is utilized as all or part of the non-oxygenated (and/or non-reductive) atmosphere pumped into the vaporization chamber. Particularly with regard to certain strains of cannabis (such as certain strains in the Indica group), cannabis may be sedating. For medical use, it may be desirable to utilize Xenon to cause initial sedation while the sedating effects of the cannabis take effect. By the time the Xenon sedation abates, in many cases the patient will be asleep, and the sleep maintained by the sedating effects of the cannabis.

Thus, in certain aspects, an apparatus for inducing sleep may comprise (i) a vaporization chamber for placement of one or more sedating substances capable of becoming at least partially vaporized, the vaporization chamber sealed except for two or more conduits, (ii) a first conduit of the two or more conduits operably coupled to a first source of a first gas that is comprised of Xenon, (iii) a heating element capable of heating the vaporization chamber; and (iv) a second conduit of the two or more conduits, the second conduit configured to transport vaporization gases and vaporized elements of the one or more sedating substances out of the vaporization chamber.

The vaporization chamber may contain an amount of oxygen below a limiting oxygen concentration (LOC) for combustion of materials within the vaporization chamber, and the one or more sedating substances may comprise cannabis or a benzodiazepine.

In another aspect, it may be desirable to cause slight hypoxia. This may be done to make the “high” come on faster, to sedate the user (particularly in the case of sleep issues), or for other reasons. In such a case, it would be desirable that the gas or gas mixture inhaled by the user contains less than a set amount of oxygen. In one aspect, the first several breaths may have less oxygen than subsequent breaths. It should be noted that oxygen may be introduced proximal to the vaporization chamber, such as at position 106.

The medication administered in combination with cannabis need not be Xenon, as there are many other medications. For certain medications, it is possible to include them in the gas that transits the vaporization chamber. Indeed, even solid or liquid medications or other biologically active elements (such as caffeine) may be vaporized along with the plant matter (or alone or in combination with other biologically active elements) in order to provide a more rapid absorption of the biologically active elements. In other situations, it may be desirable to add the medication (which would often be a gas or inhalable powder) in the proximal portion of the apparatus, avoiding the heating process.

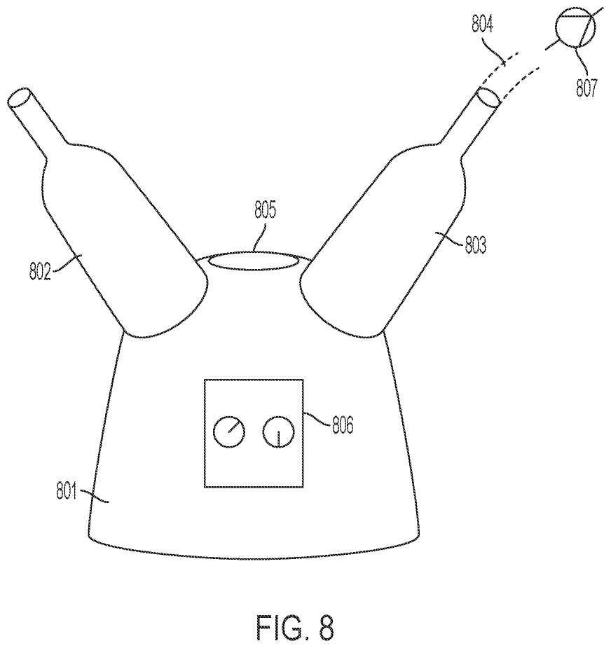

In another aspect, we teach what we call Cold Vaporization. FIG. 8 describes a cold vaporizer:

801 is a vaporization chamber.

802 is an optional breathing or output tube.

803 is a port operably connected via 804 to a vacuum device (pump) 807 capable of reducing the pressure within the chamber 801. In some aspects 802 and 803 are substantially similar in shape.

804 is a conduit through which gasses may be evacuated from the vaporization chamber 801.

805 is an optional access port through which materials may be added to the vaporization chamber 801.

806 is an optional set of mechanical or digital gauges or data providers; in one aspect it measures one or both of temperature or pressure.

807 is a vacuum device (pump).

Not shown in FIG. 8 is an optional heat source. The heat source is optional where the boiling point, at the lower pressure within the chamber 801 of the matter to be vaporized is lower than or equal to the ambient temperature.

A vaporization chamber 801 is utilized. The atmospheric pressure in that chamber is reduced by evacuating gas from the chamber 801 via a port 803 connected to an optional conduit 804, which is connected to a vacuum provider (pump) 807. By reducing the atmospheric pressure, boiling points of various substances are also reduced. With some substances, mere reduction in pressure may be enough to initiate boiling off without the addition of non-ambient heat. In other cases, a combination of heat and lower pressure may be utilized.

Varying the combination of one or more of pressure, gas and temperature may be utilized to alter the vaporization process. For example, it may be desirable to increase the boiling temperature for certain compounds so that changes that occur at higher temperatures may take place prior to boiling off. It may also be desirable to vaporize materials using atmospheric air without risking combustion by lowering the air pressure within the chamber, thereby reducing the amount of oxygen within the chamber to below the pressure-altered LOC.

When altering pressure within the chamber (and/or in conjunction with the various inventions described herein), it may be desirable to confirm that the concentration of particulates that have been vaporized meets or exceeds a target. In one respect, this solves the problem of having to repeatedly alter the pressure within a chamber, heat up the contents, restore ambient pressure (after reducing the temperature to below combustion for that pressure and oxygen concentration), inhale or otherwise dispose of the vaporized material, and then repeat the process while plant matter remains incompletely vaporized. By measuring the particulate matter in the atmosphere within the chamber (such as by measuring the dimming of a laser, measuring opacity, etc.), the vaporization process may continue until a desired concentration has been reached.

Thus, an apparatus for vaporizing vaporization targets, including but not limited to plant matter may comprise (i) a vaporization chamber, (ii) a pump for removal of an amount of gas from the chamber, wherein the apparatus is calibrated to remove enough gas to lower the boiling point of one or more substances within the chamber to a temperature at or below a set temperature and wherein the apparatus, upon actuation, lowers a pressure to match the temperature that the vaporization chamber is at or will be heated to, the apparatus equalizing the pressure after boiling to substantially match ambient atmospheric pressure; and (iii) a gas-bearing connector capable of connecting to the vaporization chamber and allowing the gas to be inhaled.

The apparatus may further comprise a heating element capable of raising the temperature within the vaporization chamber. The apparatus may also comprise a computer programmed to calculate an optimum combination of temperature and pressure for vaporization of a given substance, and to actuate pressure reduction and/or heating elements.

In some aspects, the gas remaining in the chamber may be non-oxygenated, while in other aspects, the gas remaining in the chamber is below a LOC for the pressure within the chamber.

We turn now to FIG. 4, a dual-temperature vaporizer. We note that while we use two temperatures, it is easily expanded to three or more temperature areas if needed.

101, 102, 105-111 and 114 are as described for FIG. 1 above (see paragraphs [0021], [0022], [0025]-[0031] and [0034] respectively).

403 is a dual heating chamber having a secondary vaporization chamber 413 and a primary vaporization chamber 414. Note that while both temperature areas are shown within 403, they may also be built separately and connected via a gas conduit.

405, 406, 407 and 408 are optional backflow prevention valves.

413 and 414 are, respectively, secondary and primary vaporization chambers.

415 and 416 are heating elements, one in each heating chamber.

418 is a wall separating the two zones of the heating chamber.

Although what follows is not a comprehensive list, the list of medications following this paragraph may be inserted into the process, either proximally, distally, or otherwise. For compounds that are created by heat, it may be desirable to have a secondary heating chamber and/or to include the components that must be combined for such creation. For medications subject to anaerobic pyrolysis at a level that would impair efficacy and/or create undesired byproducts, it is likely preferable to insert them into the air stream proximal to the vaporization chamber 403. In one aspect, there may be a secondary, lower-temperature vaporization chamber 413 that may vaporize the medication without passing the medication through the higher temperature chamber. In another aspect, the matter materially susceptible to pyrolysis may be largely vaporized in the primary vaporization chamber 414 at a lower temperature, and after a sufficient time period to permit the desired amount of vaporization of medication, the temperature may be increased to vaporize plant matter or pharmaceuticals (if such matter is present). The medications (including the classes of medications that the medications listed are members of) that this may be used for including, without limitation:

TABLE OF MEDICATIONS

| | |
|---|---|
| # | (Morpholinothio)Benzothiazole; 2-Phenylbenzimidazole-5-Sulfonic Acid Tea |
| | Slt; 3-(Trimethoxysilyl)Propyl Dimethyl Octadecyl Ammonium Chloride |
| A | A/New Caledonia/20/99(H1n1)-Like Strain; A/Panama/2007/99 (H3n2) -Like |
| | Strain; Abacavir (Abacavir Sulfate); Abacavir Sulfate; Abacavir Sulfate: |
| | Dolutegravir Sodium: Lamivudine; Abacavir Sulfate: Lamivudine; Abacavir |
| | Sulfate: Lamivudine: Zidovudine; Abaloparatide; Abamectin; Abatacept; |
| | Abciximab; Abemaciclib; Abies Nigra; Abiraterone Acetate; |
| | Abobotulinumtoxina; Absinthium; Acacia; Acalypha Indica; Acamprosate |
| | Calcium; Acarbose; Acebutolol (Acebutolol Hydrochloride); Acebutolol |
| | Hydrochloride; Acellular Pertussis Vaccine; Acenocoumarol; Acepromazine |
| | (Acepromazine Maleate); Acepromazine Maleate; Acetaldehyde; |
| | Acetaminophen; Acetaminophen: Benzhydrocodone Hydrochloride; |
| | Acetaminophen: Butalbital: Caffeine: Codeine Phosphate; Acetaminophen: |
| | Oxycodone Hydrochloride; Acetaminophen: Propoxyphene Napsylate; |
| | Acetaminophen: Tramadol Hydrochloride; Acetanilide; Acetarsol; Acetate |
| | (Sodium Acetate); Acetazolamide; Acetazolamide (Acetazolamide Sodium); |
| | Acetic Acid; Acetohexamide; Acetone; Acetyl-L-Tyrosine; Acetylcholine |
| | Chloride; Acetylcysteine; Acetylene; Acetylsalicylic Acid; Achillea Millefolium |
| | L.; Achyranthes Bidentata; Achyranthes Calea; Acitretin; Aclidinium Bromide; |
| | Aconite; Aconitinum; Aconitum Ferox; Aconitum Lycoctonum; Aconitum |
| | Radix; Acriflavine; Acriflavinium Chloride; Actaea Spicata; Acth; Acyclovir; |
| | Acyclovir (Acyclovir Sodium); Adalimumab; Adalimumab-Adbm; |
| | Adalimumab-Atto; Adamas; Adapalene; Adefovir Dipivoxil; Adenine; |
| | Adenophora; Adenosine; Adenovirus Vaccine Live Oral Type-4; Adenovirus |
| | Vaccine Live Oral Type-7; Adipic Acid; Adonis Vernalis; Adrenal Cortical |
| | Extract; Adrenal Tissue; Aesculus Carnea; Aesculus Glabra; Aesculus |
| | Hippocastanum; Aesculus Hippocastanum Flos; Aesculus Indica; Aethusa |
| | Cynapium; Afatinib (Afatinib Dimaleate); Aflibercept; Afoxolaner; Agalsidase |
| | Alfa; Agalsidase Beta; Agar; Agaricinum; Agaricus; Agaricus Campanulatus; |
| | Agaricus Campestris; Agaricus Emeticus; Agaricus Muscarius; Agaricus |
| | Pantherinus; Agaricus Phalloides; Agaricus Procerus; Agastache Rugosa; Agave |
| | Americana; Agnus Castus; Agraphis Nutans; Agrimonia Odorata Flos; |
| | Agrimony; Agrostemma Githago; Air; Alanine; Albendazole; Albiglutide; |
| | Albumen; Albumin; Albumin (Albumin (Human)); Albumin (Human); Albumin |
| | Radioiodinated I-125; Albumin Tannate; Albutrepenonacog Alfa; Alchemilla |
| | Vulgaris; Alcloxa; Alcohol Anhydrous; Alcohol Denatured; Alder; Alder |
| | Buckthorn; Aldesleukin; Aldioxa; Alectinib (Alectinib Hydrochloride); |
| | Alefacept; Alemtuzumab; Alendronate Sodium; Alendronate Sodium: |
| | Cholecalciferol; Alendronic Acid (Alendronate Sodium Trihydrate); Alendronic |
| | Acid (Alendronate Sodium); Aletris Farinosa; Alexidine Dihydrochloride; Alfa |
| | 1-Proteinase Inhibitor (Human); Alfacalcidol; Alfalfa; Alfaxalone; Alfentanil |
| | (Alfentanil Hydrochloride); Alfuzosin Hydrochloride; Alginic Acid; |
| | Alglucosidase Alfa; Alirocumab; Aliskiren (Aliskiren Fumarate); Alisma |
| | Plantago; Alitretinoin; Alkoxypolyethoxyethanol Iodized Complex; Alkyl C9- |
| | C15 Tolyl Methyl Trimethyl Ammonium Chlori; Alkyl Dimethyl Benzyl |
| | Ammonium Saccharinate; Alkyl Dimethyl Ethyl Ammonium Bromide; Alkyl |
| | Dimethyl Ethylbenzyl Ammonium Chloride; Alkyl N-Ethyl Morpholinium |
| | Ethylsulfates; Alkyl Phenol Polyglycol Ether Iodine Complex; Allantoin; |
| | Allergen Extract - Alum Precipitated; Allergen Extract - Diagnostic; Allergenic |
| | Extract Non-Pollen; Allergenic Extracts; Allergenic Extracts Pollens; |
| | Allopurinol; Allopurinol: Lesinurad; Alloxanum; Allylisothiocyanate; Almond; |
| | Almotriptan (Almotriptan Malate); Alnus Glutinosa; Aloe; Aloe (Aloe Ferox); |
| | Aloe Vera; Alogliptin (Alogliptin Benzoate); Alogliptin Benzoate; Alogliptin |
| | Benzoate: Metformin Hydrochloride; Alogliptin Benzoate: Pioglitazone |
| | Hydrochloride; Aloin; Aloin (Aloe); Alosetron Hydrochloride; Alpha Linoleic |
| | Acid (Flaxseed); Alpha-Galactosidase; Alpha-Globulin; Alpha-Linolenic Acid; |
| | Alpha-Linolenic Acid (Evening Primrose Oil); Alpha-Linolenic Acid (Flaxseed); |
| | Alpha-Linolenic Acid (Linseed Oil); Alpha-Linolenic Acid (Linum |
| | Usitatissimum); Alpha-Linolenic Acid (Soybean Oil); Alprazolam; Alprostadil; |
| | Alstonia Constricta; Alstonia Scholaris; Alteplase; Althaea Officinalis; |
| | Altrenogest; Altretamine; Aluminum; Aluminum Acetate; Aluminum |
| | Ammonium Sulfate; Aluminum Ammonium Sulfate Dodecahydrate; Aluminum |
| | Chloride; Aluminum Chlorohydrate; Aluminum Diacetate; Aluminum |
| | Gluconate; Aluminum Hydroxide; Aluminum Hydroxide (Dried Aluminum |
| | Hydroxide Gel); Aluminum Hydroxide-Magnesium Carbonate-Co Dried Gel; |
| | Aluminum Hydroxide-Sucrose Dried Gel; Aluminum Oxide; Aluminum |
| | Potassium Sulfate; Aluminum Sesquichlorohydrate; Aluminum Silicate; |
| | Aluminum Sodium Sulfate; Aluminum Sulfate; Aluminum Zirconium |
| | Octachlorohydrex Gly; Aluminum Zirconium Pentachlorohydrex-Glycine; |
| | Aluminum Zirconium Tetrachlorohydrex Gly; Aluminum Zirconium |
| | Trichlorohydrex Glycine; Aluminum Zirconyl Hydroxy Chloride; Aluminum |
| | Zirconyl Hydroxychloride Complex; Amantadine Hydrochloride; Amber; Ambra |
| | Grisea; Ambrisentan; Amcinonide; Amifampridine; Amifampridine Phosphate; |
| | Amifostine; Amikacin (Amikacin Sulfate); Amikacin Sulfate; Amiloride |
| | Hydrochloride; Aminacrine Hydrochloride; Aminobenzoic Acid; |
| | Aminoglutethimide; Aminohippurate Sodium; Aminolevulinic Acid |
| | Hydrochloride; Aminophylline; Amiodarone Hydrochloride; Amitraz; |
| | Amitriptyline (Amitriptyline Hydrochloride); Amitriptyline Hydrochloride; |
| | Amitriptyline Hydrochloride: Chlordiazepoxide; Amitriptyline Pamoate; |
| | Amlexanox; Amlodipine (Amlodipine Besylate); Amlodipine (Amlodipine |
| | Mesylate); Ammi Visnaga; Ammonia; Ammoniacum; Ammoniacum Gummi; |
| | Ammonium Acetate; Ammonium Benzoicum; Ammonium Bicarbonate; |
| | Ammonium Bromide; Ammonium Carbonate; Ammonium Chloride; |
| | Ammonium Citrate; Ammonium Hydroxide; Ammonium Iodide; Ammonium |
| | Lauryl Sulfate Iodine Complex; Ammonium Meta Vanadate; Ammonium |
| | Nitrate; Ammonium Phosphate; Ammonium Phosphate Dibasic; Ammonium |
| | Picricum; Ammonium Tartrate; Ammonium Valerate; Amobarbital Sodium; |
| | Amoxapine; Amoxicillin; Amoxicillin (Amoxicillin Trihydrate); Amoxicillin |
| | (Amoxicillin); Amoxicillin Trihydrate; Ampelopsis Quinquefolia; |
| | Amphetamine; Amphetamine Aspartate: Amphetamine Sulfate: |
| | Dextroamphetamine Saccharate: Dextroamphetamine Sulfate; Amphetamine |
| | Sulfate; Amphotericin B; Ampicillin; Ampicillin (Ampicillin Sodium); |
| | Ampicillin (Ampicillin Trihydrate); Ampicillin Sodium; Amprenavir; |
| | Amprolium; Amsacrine; Amygdala Amara; Amygdalae Amarae Oleum; |
| | Amygdalus Persica; Amyl Cinnamal; Amyl Nitrite; Amylase; Amylase |
| | (Aspergillus Niger); Amylase (Aspergillus Oryzae); Amylmetacresol; |
| | Amylocaine Hydrochloride; Amylopsin; Anacardium Occidentale; Anacardium |
| | Orientale; Anagallis Arvensis; Anagrelide (Anagrelide Hydrochloride |
| | Monohydrate); Anagrelide (Anagrelide Hydrochloride); Anakinra; Anastrozole; |
| | Anatherum Muricatum; Ancestim; Anchusa Officinalis; Ancrod; Anemarrhena |
| | Rhizome; Anemone Nemorosa; Anemopsis Californica; Anethole; Anethole |
| | Trithione; Angelica; Angelica Root; Angelicae Sinensis Radix; Angustura Vera; |
| | Anidulafungin; Anileridine (Anileridine Hydrochloride); Anileridine |
| | (Anileridine Phosphate); Aniline; Animal Ext Homeopathic; Anise; Anise |
| | (Pimpinella Anisum); Anistreplase; Antazoline Phosphate; Antazoline Sulfate; |
| | Anti-D (Rh) Immunoglobulin (Human); Anti-Inhibitor Coagulant Complex; |
| | Anti-Thymocyte Globulin (Rabbit); Antihemophilic Factor (Human); |
| | Antihemophilic Factor (Recombinant; Antihemophilic Factor (Recombinant |
| | Bdd); Antihemophilic Factor (Recombinant); Antihemophilic Factor |
| | (Recombinant) Porcine Sequence; Antimony; Antimony Pentasulfide; Antimony |
| | Potassium Tartrate; Antimony Trichloride; Antimony Triiodide; Antimony |
| | Trisulfide; Antipyrine; Antithrombin Iii (Human); Antivenin Crotaline |
| | Polyvalent; Apalutamide; Apiol; Apis Mellifera Venom Protein Extract; Apis |
| | Mellifica; Apis Venenum Purum; Apixaban; Apocynum Androsaemifolium; |
| | Apocynum Cannabinum; Apomorphine; Apomorphine Hydrochloride; |
| | Apraclonidine (Apraclonidine Hydrochloride); Apramycin (Apramycin Sulfate); |
| | Apremilast; Aprepitant; Apricot; Aprotinin; Aprotinin (Bovine); Aprotinin |
| | (Synthetic); Aquilaria Agallocha; Aquilegia Vulgaris; Arachidonic Acid; Aralia |
| | Racemosa; Arbutin; Arbutus Andrachne; Arcitumomab; Areca Catechu; |
| | Arecoline Hydrobromide; Arformoterol Tartrate; Argatroban; Argemone |
| | Mexicana; Argentum Iodatum; Argentum Muriaticum; Argentum Oxydatum; |
| | Arginine; Arginine Hydrochloride; Aripiprazole; Aripiprazole Lauroxil; |
| | Aristolochia Milhomens; Aristolochia Serpentaria; Armodafinil; Arnica; Arnica |
| | Montana; Arsanilic Acid; Arsenic Trioxide; Arsonoacetic Acid; Artemisia |
| | Abrotanum; Articaine Hydrochloride; Arum Dracontium; Arum Italicum; Arum |
| | Triphyllum; Arundo Mauritanica; Asafetida; Asari Cum Radice; Asarum; |
| | Asarum Europaeum; Asbestos; Asclepias Incarnata; Asclepias Syriaca; |
| | Asclepias Tuberosa; Asclepias Vincetoxicum; Ascorbic Acid: Polyethylene |
| | Glycol 3350: Potassium Chloride: Sodium Ascorbate: Sodium Chloride: Sodium |
| | Sulfate; Ascorbyl Palmitate; Asenapine (Asenapine Maleate); Asfotase Alfa; |
| | Asparaginase; Asparagus Officinalis; Aspergillus Niger; Aspergillus Oryzae; |
| | Asperula Odorata; Aspidium; Aspidosperma; Aspirin: Butalbital: Caffeine: |
| | Codeine Phosphate; Aspirin: Caffeine: Dihydrocodeine Bitartrate; Aspirin: |
| | Omeprazole; Aspirin: Oxycodone Hydrochloride; Aspirin: Oxycodone |
| | Hydrochloride: Oxycodone Terephthalate; Astacus Fluviatilis; Astemizole; |
| | Aster; Asterias Rubens (Homeo); Astragalus; Asunaprevir; Atazanavir |
| | (Atazanavir Sulfate); Atenolol; Atezolizumab; Atipamezole Hydrochloride; |
| | Atomoxetine (Atomoxetine Hydrochloride); Atomoxetine Hydrochloride; |
| | Atorvastatin (Atorvastatin Calcium Propylene Glycol Solvate); Atorvastatin |
| | (Atorvastatin Calcium); Atovaquone; Atractylodes Macrocephala; Atracurium |
| | Besylate; Atropine; Atropine Methylnitrate; Atropine Sulfate; Attapulgite |
| | (Activated); Auranofin; Aurothioglucose; Avelumab; Avilamycin; Avobenzone; |
| | Axicabtagene Ciloleucel; Axitinib; Azacitidine; Azadirachta Indica; Azaperone; |
| | Azatadine Maleate; Azathioprine; Azathioprine (Azathioprine Sodium); Azelaic |
| | Acid; Azelastine Hydrochloride; Azilsartan Medoxomil (Azilsartan Medoxomil |
| | Potassium); Azithromycin; Azithromycin (Azithromycin Dihydrate); |
| | Azithromycin (Azithromycin Hydrogen Citrate); Azithromycin (Azithromycin |
| | Isopropanolate Monohydrate); Azithromycin (Azithromycin Monohydrate |
| | Hemiethanolate); Azithromycin (Azithromycin Monohydrate); Aztreonam |
| B | B/Hong Kong/1434/2002-Like Strain; Bacampicillin Hydrochloride; Bacillus |
| | Calmette-Guerin (Bcg) Strain Tice; Bacillus Calmette-Guerin (Bcg) Substrain |
| | Connaught; Bacillus Calmette-Guerin (Bcg) Substrain Montreal; Bacillus |
| | Calmette-Guerin Bcg; Bacitracin; Bacitracin (Bacitracin Methylene |
| | Disalicylate); Bacitracin (Bacitracin Zinc); Bacitracin Zinc; Baclofen; Bacterials; |
| | Badiaga; Baja; Balloon Flower; Ballota Nigra; Balm Oil; Balmony; Balsam |
| | Mecca; Bambermycin; Baptisia Tinctoria; Barberry Root; Baricitinib; Barium |
| | Carbonate; Barium Chloride; Barium Sulfate; Barley; Baryta Acetica; Baryta |
| | Iodata; Basil (Ocimum Basilicum); Basiliximab; Bayberry; Bazedoxifene |
| | (Bazedoxifene Acetate); Bear's Breech; Becaplermin; Becaplermin (Rhpdgf-Bb); |
| | Beclomethasone Dipropionate; Bedaquiline Fumarate; Beechwood Creosote; |
| | Beef Extract Powder; Beeswax; Beet; Belatacept; Belimumab; Belladonna; |
| | Belladonna Radix; Bellis Perennis; Bemotrizinol; Benazepril Hydrochloride; |
| | Bendamustine Hydrochloride; Bendroflumethiazide; Benoxinate Hydrochloride; |
| | Benralizumab; Benserazide (Benserazide Hydrochloride); Bentonite; |
| | Benzalkonium Chloride; Benzene; Benzethonium Chloride; Benzinum |
| | Dinitricum; Benzocaine; Benzoic Acid; Benzoin; Benzoin Odoriferum; |
| | Benzophenone; Benzoyl Peroxide; Benztropine Mesylate; Benzydamine |
| | Hydrochloride; Benzyl Alcohol; Benzyl Benzoate; Benzyl Di-Hydroxyethyl- |
| | Cocoalkyl-Ammonium Chl; Benzyl Paraben; Benzylpenicilloyl-Polylysine; |
| | Bepotastine Besilate; Beractant; Berberis Aquifolium; Berberis Vulgaris Fructus; |
| | Beryllium Metallicum; Besifloxacin (Besifloxacin Hydrochloride); Beta |
| | Globulin; Beta-Carotene; Beta-Carotene (Beta-Carotene); Beta-Carotene |
| | (Dunaliella Salina); Beta-Carotene (Provitamin A); Beta-Carotene (Vitamin A); |
| | Beta-Galactosidase; Betaglucanase; Betahistine Hydrochloride; Betaine; Betaine |
| | Hydrochloride; Betamethasone; Betamethasone (Betamethasone 21-Disodium |
| | Phosphate); Betamethasone (Betamethasone Dipropionate); Betamethasone |
| | (Betamethasone Sodium Phosphate); Betamethasone (Betamethasone Valerate); |
| | Betamethasone Acetate; Betamethasone Benzoate; Betamethasone Dipropionate; |
| | Betamethasone Sodium Phosphate; Betamethasone Valerate; Betanaphthol; |
| | Betaxolol (Betaxolol Hydrochloride); Bethanechol Chloride; Betiatide; |
| | Betrixaban; Betula Pendula; Betula Pendula (Roth); Betula Pendula Folia; |
| | Bevacizumab; Bezafibrate; Bicalutamide; Bicarbonate (Sodium Bicarbonate); |
| | Bicisate Dihydrochloride; Bictegravir (Bictegravir Sodium); Bifidobacterium |
| | Bifidum; Bifidobacterium Longum; Bilastine; Bile Salts; Bimatoprost; |
| | Bioallethrin; Bioflavonoids; Biotin; Biotin (D-Biotin); Biotin (Yeast); Biperiden |
| | Hydrochloride; Birthwort; Bis(1-Piperidylthiocarbonyl)Disulfide; Bisacodyl; |
| | Bisacodyl: Polyethylene Glycol 3350: Potassium Chloride: Sodium Bicarbonate: |
| | Sodium Chloride; Bismuth; Bismuth Camphocarbonate; Bismuth |
| | Dipropylacetate; Bismuth Oxide (Biskalcitrate Potassium); Bismuth |
| | Subcarbonate (Oxycarbonate); Bismuth Subgallate; Bismuth Subnitrate; Bismuth |
| | Subsalicylate; Bismuth Tribromophenate; Bisoctrizole; Bisoprolol Fumarate; |
| | Bitter Orange; Bitter Orange (Citrus Aurantium L.); Bivalirudin; Black Alder; |
| | Black Currant; Black Pepper; Black Widow Spider Antivenin; Blackberry Leaf; |
| | Blackroot; Blatta Americana; Blatta Orientalis; Bleomycin (Bleomycin Sulfate); |
| | Bleomycin Sulfate; Blessed Thistle; Blessed Thistle (Cnicus Benedictus); |
| | Blinatumomab; Boceprevir; Boldenone Undecylenate; Boldo; Boldo Alkaloid; |
| | Boletus Luridus; Boletus Satanus; Bombyx Processionea; Bone Marrow; Bone |
| | Meal; Borago Officinalis; Boric Acid; Borneol; Bortezomib (Bortezomib |
| | Mannitol Boronic Ester); Bosentan; Bosentan (Bosentan Monohydrate); |
| | Bosutinib (Bosutinib Monohydrate); Boswellia Serrata; Botulinum Antitoxin; |
| | Botulinum Toxin Type B; Bovine Ovaries; Bovine Plasma; Bovista; Box Elder; |
| | Brain; Brassica Napus; Braunite; Brayera; Brentuximab Vedotin; Bretylium |
| | Tosylate; Brexanolone; Brexpiprazole; Brigatinib; Brilliant Green; Brimonidine |
| | (Brimonidine Tartrate); Brimonidine Tartrate; Brinzolamide; Brivaracetam; |
| | Brodalumab; Bromazepam; Bromelain; Bromelain (Pineapple Enzyme); |
| | Bromelain (Pineapple); Bromfenac (Bromfenac Sodium Sesquihydrate); |
| | Bromine; Bromine-Phenol-Benzethonium Chloride Complex; Bromocriptine |
| | (Bromocriptine Mesylate); Bromodiphenhydramine Hydrochloride; |
| | Brompheniramine Maleate; Bromus Ramosus; Bronopol; Brucine; Bryonia; |
| | Buchu; Buckthorn; Buckwheat; Budesonide; Budesonide: Formoterol Fumarate |
| | Dihydrate; Bufexamac; Buffer Solution; Bufo Vulgaris; Bumetanide; |
| | Bunamidine Hydrochloride; Bupivacaine Hydrochloride; Bupleurum Chinense |
| | Dc; Buprenorphine; Buprenorphine (Buprenorphine Hydrochloride); |
| | Buprenorphine Hydrochloride; Buprenorphine Hydrochloride: Naloxone |
| | Hydrochloride; Bupropion Hydrobromide; Bupropion Hydrochloride; Bupropion |
| | Hydrochloride: Naltrexone Hydrochloride; Burdock; Burnet; Burosumab; |
| | Burweed; Buserelin (Buserelin Acetate); Buserelin Acetate; Buspirone |
| | Hydrochloride; Busulfan; Butabarbital Sodium; Butacaine Sulfate; Butalbital; |
| | Butamben; Butanediol; Butcher's Broom; Butenafine Hydrochloride; Buthus |
| | Australis; Butoconazole Nitrate; Butorphanol (Butorphanol Tartrate); |
| | Butorphanol Tartrate; Butoxy Polyoxypropylene Polyoxyethylene Gly. Iodine; |
| | Butoxypolypropylene Glycol; Butyl Alcohol; Butyl Carbitol; Butylparaben; |
| | Butyric Acid; Buxus Sempervirens |
| C | C1 Esterase Inhibitor (Human); Cabazitaxel; Cabergoline; Cabozantinib |
| | (Cabozantinib Malate); Cactus Grandiflorus; Cadmium Bromide; Cadmium |
| | Iodatum; Cadmium Metallicum; Cadmium Oxide; Cadmium Sulfide; Cadmium |
| | Sulphuricum; Caffeine; Caffeine (Caffeine Citrate); Caffeine Citrate; Cahinca; |
| | Calabar Bean; Caladium Seguinum; Calamine; Calamus; Calcifediol; |
| | Calcipotriol; Calcipotriol (Calcipotriol Monohydrate); Calcitonin (Salmon |
| | Synthetic); Calcitriol; Calcium; Calcium (Bone Meal; Calcium (Bone Meal); |
| | Calcium (Calcium Acetate); Calcium (Calcium Amino Acid Chelate); Calcium |
| | (Calcium Ascorbate; Calcium (Calcium Ascorbate); Calcium (Calcium |
| | Borogluconate; Calcium (Calcium Borogluconate); Calcium (Calcium |
| | Carbonate; Calcium (Calcium Carbonate); Calcium (Calcium Chloride); Calcium |
| | (Calcium Citrate; Calcium (Calcium Citrate); Calcium (Calcium Fumarate; |
| | Calcium (Calcium Glucoheptonate; Calcium (Calcium Gluconate; Calcium |
| | (Calcium Gluconate); Calcium (Calcium Glycerophosphate); Calcium (Calcium |
| | Hvp Chelate; Calcium (Calcium Hvp Chelate); Calcium (Calcium |
| | Hydroxyapatite; Calcium (Calcium Hydroxyapatite); Calcium (Calcium Lactate; |
| | Calcium (Calcium Lactate); Calcium (Calcium Lactobionate; Calcium (Calcium |
| | Maleate; Calcium (Calcium Organic Complex); Calcium (Calcium Phosphate; |
| | Calcium (Calcium Phosphate (Dibasic); Calcium (Calcium Phosphate (Dibasic)); |
| | Calcium (Calcium Phosphate (Tribasic); Calcium (Calcium Phosphate |
| | (Tribasic)); Calcium (Calcium Phosphate Monobasic; Calcium (Calcium |
| | Phosphate Monobasic); Calcium (Calcium Phosphate); Calcium (Calcium |
| | Proteinate; Calcium (Calcium Proteinate); Calcium (Dicalcium Orthophosphate); |
| | Calcium (Dolomite; Calcium (Dolomite); Calcium (Durapatite); Calcium (Egg; |
| | Calcium (Egg Shell); Calcium (Hydroxyapatite); Calcium (Leucovorin |
| | Calcium); Calcium (Oyster Shells; Calcium (Oyster Shells); Calcium (Oyster); |
| | Calcium Acetate; Calcium Alginate; Calcium Amino Acid Chelate; Calcium |
| | Ascorbate; Calcium Aspartate; Calcium Bisdioctylsulfosuccinate; Calcium |
| | Borogluconate; Calcium Carbimide; Calcium Carbonate; Calcium Carbonate |
| | (Dolomite); Calcium Carbonate (Oyster Shells); Calcium Carbonate: Risedronate |
| | Sodium; Calcium Caseinate; Calcium Chloride; Calcium Chloride (Calcium |
| | Chloride); Calcium Citrate; Calcium D-Pantothenate; Calcium D-Pantothenate |
| | (Menadione Sodium Bisulfite); Calcium Fluoride; Calcium Glucoheptonate; |
| | Calcium Gluconate; Calcium Gluconate Monohydrate; Calcium Gluconolactate; |
| | Calcium Glycerophosphate; Calcium Hexafluorosilicate; Calcium Hvp Chelate; |
| | Calcium Hvp Chelate (Bone Meal); Calcium Hydroxide; Calcium Hypochlorite; |
| | Calcium Hypophosphite; Calcium Iodate; Calcium Iodide; Calcium Lactate; |
| | Calcium Lactobionate; Calcium Lactophosphate; Calcium Levulinate; Calcium |
| | Magnesium Phytate; Calcium Oxalate; Calcium Phosphate (Dibasic); Calcium |
| | Phosphate (Tribasic); Calcium Picrate; Calcium Polycarbophil; Calcium |
| | Polystyrene Sulfonate; Calcium Propionate; Calcium Proteinate; Calcium |
| | Sennosides A & B; Calcium Silicate; Calcium Sulfate; Calcium Sulfide Crude; |
| | Calcium Undecylenate; Calendula; Calluna Vulgaris; Calotropis Gigantea; |
| | Caltha Palustris; Calumba; Camphene; Camphor; Camphor Monobromated; |
| | Camphoricum Acidum; Canada Balsam; Canagliflozin; Canagliflozin: |
| | Metformin Hydrochloride; Canakinumab; Canchalagua; Candesartan Cilexetil; |
| | Candida Albicans Antigen; Candida Parapsilosis; Cannabidiol; Cannabidiol |
| | (Cannabis Sativa Extract); Cantharides; Cantharidin; Capecitabine; Capreomycin |
| | (Capreomycin Sulfate); Capromab Pendetide; Caprylic/Capric Acid Monoesters; |
| | Caprylycollagenic Acid; Capsaicin; Capsaicin (Capsicum Oleoresin); Capsaicin |
| | (Capsicum); Capsicum; Capsicum Oleoresin; Captopril; Caraway; Carbachol; |
| | Carbadox; Carbamazepine; Carbaryl; Carbetapentane Citrate; Carbetocin; |
| | Carbidopa; Carbidopa (Carbidopa Monohydrate); Carbo Animalis; |
| | Carbohydrate; Carbomer; Carbon Black; Carbon Dioxide; Carbon Disulfide; |
| | Carbon Monoxide; Carbon Tetrachloride; Carboneum Hydrogenisatum; |
| | Carboplatin; Carboprost (Carboprost Tromethamine); Carboxymethylcellulose |
| | Sodium; Carfentanil (Carfentanil Citrate); Carfilzomib; Carglumic Acid; |
| | Cariprazine Hydrochloride; Carisoprodol; Carmustine; Carnidazole; Carpinus |
| | Betulus; Carprofen; Carrot; Carvacrol; Carvedilol; Carya Alba; Casanthranol; |
| | Cascara; Cascara Sagrada; Cascarilla; Casein; Casein Hydrolysate; Caspofungin |
| | (Caspofungin Acetate); Cassia Alata; Cassia Fistula; Castanea Sativa; Castanea |
| | Vesca; Castor Equi; Castor Oil; Castoreum; Cat (Felis Domesticus) Pelt And Cat |
| | Dander; Cat Fur; Cat Thyme; Catalpa Bignonioides; Catnep; Catridecacog; |
| | Catumaxomab; Caulophyllum Thalictroides; Cedron; Cedrus Libani; Cefaclor; |
| | Cefadroxil; Cefadroxil (Cefadroxil Monohydrate); Cefamandole (Cefamandole |
| | Nafate); Cefamandole Nafate; Cefazolin (Cefazolin Sodium); Cefazolin Sodium; |
| | Cefepime (Cefepime Hydrochloride); Cefixime; Cefotaxime (Cefotaxime |
| | Sodium); Cefotaxime Sodium; Cefotetan (Cefotetan Disodium); Cefotetan |
| | Di sodium; Cefovecin (Cefovecin Sodium); Cefoxitin (Cefoxitin Sodium); |
| | Cefpodoxime (Cefpodoxime Proxetil); Cefprozil; Cefprozil (Cefprozil |
| | Monohydrate); Ceftazidime; Ceftazidime (Ceftazidime Pentahydrate); Ceftiofur |
| | (Ceftiofur Crystalline Free Acid); Ceftiofur (Ceftiofur Hydrochloride); Ceftiofur |
| | (Ceftiofur Sodium); Ceftizoxime Sodium; Ceftobiprole (Ceftobiprole Medocaril |
| | Sodium); Ceftobiprole (Ceftobiprole Medocaril); Ceftolozane (Ceftolozane |
| | Sulfate); Ceftriaxone (Ceftriaxone Sodium); Cefuroxime (Cefuroxime Axetil); |
| | Cefuroxime (Cefuroxime Sodium); Cefuroxime Sodium; Celandine; Celecoxib; |
| | Celery; Celestite; Cell Wall Skeleton; Cellulase; Cellulase (Aspergillus Oryzae); |
| | Cemiplimab; Cenegermin; Centaurea Cyanus; Centaurium Umbellatum; |
| | Centella; Centipeda Minima; Cephalexin; Cephalexin (Cephalexin |
| | Monohydrate); Cephalothin (Cephalothin Sodium); Cephalothin Sodium; |
| | Cephapirin (Cephapirin Benzathine); Cephapirin (Cephapirin Sodium); |
| | Cephapirin Benzathine; Ceratostigma Willmottianum; Cereus Bonplandii; |
| | Ceritinib; Cerivastatin Sodium; Cerliponase Alfa; Cerous Oxalate; Certolizumab |
| | Pegol; Cetalkonium Chloride; Cetirizine Hydrochloride; Cetomacrogol 1000; |
| | Cetostearyl Alcohol; Cetraria Islandica; Cetrimide; Cetrimonium Bromide; |
| | Cetrorelix (Cetrorelix Acetate); Cetuximab; Cetylpyridinium Chloride; |
| | Chalcopyrite; Chamomile; Chamomilla Vulgaris; Charcoal Activated; |
| | Chelidonium; Chemicals Homeopathic; Chenopodium Ambrosioides; |
| | Chenopodium Vulvaria; Chickweed; Chimaphila Umbellata; Chinese Pagoda; |
| | Chinese Quince Fruit; Chinese Tinospora Stem; Chininum Salicylicum; Chloral |
| | Hydrate; Chlorambucil; Chloramine T.; Chloramphenicol; Chloramphenicol |
| | (Chloramphenicol Palmitate); Chloramphenicol (Chloramphenicol Sodium |
| | Succinate); Chloramphenicol Palmitate; Chlordan; Chlordiazepoxide |
| | Hydrochloride; Chlordiazepoxide Hydrochloride: Clidinium Bromide; |
| | Chlorhexidine; Chlorhexidine (Chlorhexidine Acetate); Chlorhexidine |
| | (Chlorhexidine Digluconate); Chlorhexidine Acetate; Chlorhexidine Gluconate; |
| | Chlorhexidine Hydrochloride; Chloride (Magnesium Chloride); Chloride |
| | (Potassium Chloride); Chloride (Sodium Chloride); Chlorine; Chlorine |
| | (Potassium Chloride); Chlorine (Sodium Dichloro-S-Triazinetrione); Chlorine |
| | (Sodium Hypochlorite); Chlorine (Trisodium Phosphate Chlorinated); Chlorine |
| | Dioxide; Chloro-M-Cresol; Chlorobutanol; Chloroform; Chlorophyll; |
| | Chlorophyllins; Chloroprocaine Hydrochloride; Chloroquinaldol; Chloroquine |
| | Diphosphate; Chlorothiazide; Chloroxylenol; Chlorphenesin; Chlorpheniramine; |
| | Chlorpheniramine Maleate; Chlorpheniramine Maleate: Codeine Phosphate; |
| | Chlorpheniramine Maleate: Hydrocodone Bitartrate; Chlorpheniramine Maleate: |
| | Hydrocodone Bitartrate: Pseudoephedrine Hydrochloride; Chlorpheniramine |
| | Polistirex: Codeine Polistirex; Chlorpheniramine Polistirex: Hydrocodone |
| | Polistirex; Chlorpromazine; Chlorpromazine (Chlorpromazine Hydrochloride); |
| | Chlorpromazine Hydrochloride; Chlorpropamide; Chlortetracycline; |
| | Chlortetracycline (Chlortetracycline Hydrochloride); Chlortetracycline |
| | Hydrochloride; Chlortetracycline Hydrochloride (Chlortetracycline Calcium |
| | Complex); Chlortetracycline Hydrochloride (Chlortetracycline Calcium); |
| | Chlortetracycline Sulfate; Chlorthalidone; Chlorzoxazone; Cholecystokinin; |
| | Cholera Vaccine; Cholesterol; Cholestyramine Resin; Choline; Choline (Choline |
| | Bitartrate; Choline (Choline Bitartrate); Choline (Choline Chloride); Choline |
| | (Choline Citrate); Choline (Phosphatidyl Choline); Choline Bitartrate; Choline |
| | Bitartrate (Yeast); Choline Chloride; Choline Citrate; Choline Dihydrogen |
| | Citrate; Choline Dihydrogen Citrate (Choline Bitartrate); Choline Salicylate; |
| | Choriogonadotropin Alfa; Chorionic Gonadotropin; Chromic Chloride; Chromic |
| | Glutamate; Chromic Phosphate P32; Chromic Sulfate; Chromium; Chromium |
| | (Chromic Chloride; Chromium (Chromic Chloride); Chromium (Chromic |
| | Glutamate); Chromium (Chromic Oxide); Chromium (Chromic Sulfate); |
| | Chromium (Chromium Amino Acid Chelate); Chromium (Chromium Chloride |
| | Cr51); Chromium (Chromium Citrate; Chromium (Chromium Citrate); |
| | Chromium (Chromium Glutarate); Chromium (Chromium Hvp Chelate); |
| | Chromium (Chromium Malate); Chromium (Chromium Organic Complex); |
| | Chromium (Chromium Proteinate); Chromium (Chromium Sulfate); Chromium |
| | (Chromium Trioxide); Chromium (Chromium Yeast); Chromium (Kelp; |
| | Chromium (Yeast); Chromium Aspartate; Chromium Chloride Cr51; Chromium |
| | Citrate; Chromium Hvp Chelate; Chromium Hvp Chelate (Chromic Oxide); |
| | Chromium Proteinate; Chromium Sulfate; Chromium Trioxide; Chrysanthemum |
| | Morifolia; Chrysarobin; Chymopapain; Chymotrypsin; Cicer Arietinum; |
| | Cichorium Intybus; Ciclesonide; Ciclopirox; Ciclopirox Olamine; Cicuta |
| | Maculata; Cicuta Virosa; Cidofovir (Cidofovir Dihydrate); Cilastatin (Cilastatin |
| | Sodium); Cilazapril; Cilazapril (Cilazapril Monohydrate); Cimetidine; |
| | Cimetidine (Cimetidine Hydrochloride); Cimetidine Hydrochloride; Cimex |
| | Lectularius; Cimicifuga Racemosa; Cina; Cinacalcet (Cinacalcet Hydrochloride); |
| | Cinchona Officinalis; Cinchona Succirubra; Cinnamaldehyde; Cinnamedrine; |
| | Cinnamomum Zeylanicum; Cinnamomum Zeylanicum (Cinnamomum Verum); |
| | Cinnamon Oil; Cinnamyl Alcohol; Cinoxate; Ciprocinonide; Ciprofloxacin; |
| | Ciprofloxacin (Ciprofloxacin Hydrochloride; Ciprofloxacin (Ciprofloxacin |
| | Hydrochloride Monohydrate); Ciprofloxacin (Ciprofloxacin Hydrochloride); |
| | Ciprofloxacin (Ciprofloxacin Lactate); Ciprofloxacin Hydrochloride; |
| | Ciprofloxacin: Ciprofloxacin Hydrochloride; Cis-Linoleic Acid; Cis-Linoleic |
| | Acid (Borage Oil; Cis-Linoleic Acid (Borage Oil); Cis-Linoleic Acid (Evening |
| | Primrose Oil); Cis-Linoleic Acid (Soybean Oil); Cisapride (Cisapride |
| | Monohydrate); Cisapride Monohydrate; Cisatracurium (Cisatracurium Besylate); |
| | Cisplatin; Cistus Canadensis; Citalopram (Citalopram Hydrobromide); |
| | Citalopram Hydrobromide; Citric Acid; Citric Acid Monohydrate; Citric Acid: |
| | Magnesium Oxide: Sodium Picosulfate; Citrullus Colocynthis; Citrus; Citrus |
| | Reticulata; Cladribine; Clam; Clarithromycin; Clavulanic Acid (Clavulanate |
| | Potassium); Clavulanic Acid (Potassium Clavulanate); Clemastine (Clemastine |
| | Hydrogen Fumarate); Clemastine Hydrogen Fumarate; Clematis; Clematis |
| | Erecta; Clematis Virginiana; Clematis Vitalba; Clenbuterol Hydrochloride; |
| | Clevidipine; Clidinium Bromide; Clindamycin; Clindamycin (Clindamycin 2- |
| | Palmitate Hydrochloride); Clindamycin (Clindamycin Hydrochloride); |
| | Clindamycin (Clindamycin Phosphate); Clindamycin Phosphate; Clioquinol; |
| | Clobazam; Clobetasol Propionate; Clobetasone Butyrate; Clodronate Disodium; |
| | Clodronate Disodium (Clodronate Disodium Tetrahydrate); Clofarabine; |
| | Clofedanol Hydrochloride; Clofibrate; Clomiphene Citrate; Clomipramine |
| | Hydrochloride; Clonazepam; Clonidine Hydrochloride; Clopidogrel |
| | (Clopidogrel Bisulfate); Clopidogrel Bisulfate; Clopidol; Cloprostenol |
| | (Cloprostenol Sodium); Clorazepate Dipotassium; Clorophene; Clorophene |
| | Sodium; Closantel (Closantel Sodium); Clotrimazole; Clove; Clove Oil; |
| | Cloxacillin (Cloxacillin Benzathine); Cloxacillin (Cloxacillin Sodium); |
| | Cloxacillin Benzathine; Cloxacillin Sodium; Clozapine; Cnidium; Coagulation |
| | Factor Ii (Human); Coagulation Factor Ix (Human); Coagulation Factor Ix |
| | (Recombinant); Coagulation Factor Vii (Human); Coagulation Factor X |
| | (Human); Coal Tar; Cobalt; Cobalt (Cobalt Carbonate (Basic)); Cobalt (Cobalt |
| | Gluconate); Cobalt (Cobalt Sulfate); Cobalt (Cobaltous Chloride); Cobalt |
| | (Cyanocobalamin); Cobalt Carbonate (Basic); Cobalt Gluconate; Cobalt Sulfate; |
| | Cobaltous Chloride; Cobaltous Sulfate Heptahydrate; Cobaltum Nitricum; |
| | Cobamamide; Cobicistat; Cobimetinib (Cobimetinib Fumarate); Cocaine |
| | Hydrochloride; Coccinella Septempunctata; Cocculus Indicus; Cochineal; |
| | Cocillana; Cocklebur; Cocoa; Cocoa Butter; Coconut Oil; Coconut Oil |
| | Ethanolamide; Cod Liver Oil; Codeine; Codeine (Codeine Monohydrate; |
| | Codeine (Codeine Phosphate); Codeine Phosphate; Codeine Phosphate: |
| | Phenylephrine Hydrochloride: Promethazine Hydrochloride; Codeine Phosphate: |
| | Promethazine Hydrochloride; Codeine Sulfate; Codonopsitis; Coffea Tosta; |
| | Coffee; Colchicine; Colchicum; Colesevelam Hydrochloride; Colestipol |
| | Hydrochloride; Colfosceril Palmitate; Colistin (Colistimethate Sodium); |
| | Collagen; Collagenase; Collagenase Clostridium Histolyticum; Collinsonia |
| | Canadensis; Colocasia Esculenta; Colostrum; Coltsfoot; Combretum; Comfrey; |
| | Common Arum; Common Bean Garden; Common Sagebrush; Comocladia |
| | Dentata; Conchiolinum; Condurango; Coniine; Conium Maculatum; Conjugated |
| | Estrogens; Convallaria Majalis; Copaiva Officinalis; Copper; Copper (Basic |
| | Copper Carbonate); Copper (Copper Amino Acid Chelate); Copper (Copper |
| | Citrate; Copper (Copper Citrate); Copper (Copper Diammonium Edta); Copper |
| | (Copper Fumarate); Copper (Copper Gluconate; Copper (Copper Gluconate); |
| | Copper (Copper Glutarate); Copper (Copper Hvp Chelate; Copper (Copper Hvp |
| | Chelate); Copper (Copper Malate; Copper (Copper Maleate); Copper (Copper |
| | Organic Complex); Copper (Copper Oxide); Copper (Copper Proteinate; Copper |
| | (Copper Proteinate); Copper (Copper Succinate); Copper (Copper Sulfate); |
| | Copper (Cupric Carbonate Basic); Copper (Cupric Citrate); Copper (Cupric |
| | Gluconate); Copper (Cupric Oxide); Copper (Cupric Sulfate); Copper Fumarate |
| | (Copper Fumarate); Copper Gluconate; Copper Hvp Chelate; Copper Hvp |
| | Chelate (Copper Gluconate); Copper Naphthenate; Copper Proteinate; Copper |
| | Sulfate; Coral-Bean Bark; Corallium Rubrum; Corbadrine; Corbadrine |
| | (Corbadrine Hydrochloride); Corbadrine Hydrochloride; Coriander; Coriander |
| | (Coriandrum Sativum); Corn Poppy; Cornus Alternifolia; Cornus Circinata; |
| | Cortex Albizziae Julibrissin; Cortex Magnoliae Officinalis; Cortisone Acetate; |
| | Corylus Avellana; Corynebacterium Diphtheriae Crm-197 Protein; Cosmos; |
| | Cotton Plant; Cotton Seed Oil Ext.; Cotyledon Umbilicus; Couch Grass; |
| | Coumarin; Cowslip; Cresol; Cresyl Acetate; Crisaborole; Crizotinib; Cromolyn |
| | Sodium; Crotamiton; Croton Oil; Cryofluorane; Cubeba Officinalis; Cucurbita |
| | Pepo; Cucurbita Pepo Semen; Cummin; Cupressus Lawsoniana; Cupressus |
| | Sempervirens; Cupric Acetate; Cupric Carbonate Basic; Cupric Chloride; Cupric |
| | Oxide; Cupric Sulfate Basic; Curare; Curculigo Orchioides; Cusparia Trifoliata; |
| | Cyanocobalamin Co 57; Cyclamen Europaeum L.; Cyclandelate; Cyclizine |
| | Hydrochloride; Cyclizine Lactate; Cyclobenzaprine Hydrochloride; |
| | Cyclopentamine Hydrochloride; Cyclopentolate Hydrochloride; |
| | Cyclophosphamide; Cycloserine; Cyclosporine; Cynara Scolymus; Cyperus |
| | Rotundus; Cypress; Cyproheptadine Hydrochloride; Cyproterone Acetate; |
| | Cysteamine (Cysteamine Bitartrate); Cysteamine (Cysteamine Hydrochloride); |
| | Cystine; Cytarabine; Cythioate; Cytisus Scoparius; Cytomegalovirus |
| | Immunolobulin (Human); |
| D | D&C Red No. 28; D-Alpha Tocopherol; D-Alpha Tocopheryl Acetate; D-Alpha |
| | Tocopheryl Acid Succinate; D-Pantothenic Acid; D-Pantothenic Acid (Calcium |
| | D-Pantothenate; D-Pantothenic Acid (Calcium D-Pantothenate); D-Pantothenic |
| | Acid (Dexpanthenol); D-Pantothenic Acid (Panthenol); D-Pantothenic Acid |
| | (Yeast); D-Penicillamine; Dabigatran Etexilate (Dabigatran Etexilate Mesilate); |
| | Dabigatran Etexilate Mesylate; Dabrafenib (Dabrafenib Mesylate); Dabrafenib |
| | Mesylate; Dacarbazine; Daclatasvir (Daclatasvir Dihydrochloride); Daclizumab; |
| | Daclizumab Beta; Dacomitinib (Dacomitinib Monohydrate); Dactinomycin; |
| | Dalbavancin (Dalbavancin Hydrochloride); Dalfampridine; Dalfopristin; |
| | Dalteparin Sodium; Damiana; Danaparoid Sodium; Danazol; Danofloxacin |
| | (Danofloxacin Mesylate); Danthron; Dantrolene Sodium; Dapagliflozin; |
| | Dapagliflozin (Dapagliflozin Propanediol Monohydrate); Dapagliflozin: |
| | Metformin Hydrochloride; Dapagliflozin: Metformin Hydrochloride: Saxagliptin |
| | Hydrochloride; Dapagliflozin: Saxagliptin Hydrochloride; Daphne Indica; |
| | Dapsone; Daptomycin; Daratumumab; Darbepoetin Alfa; Darifenacin |
| | (Darifenacin Hydrobromide); Darunavir (Darunavir Ethanolate); Dasabuvir |
| | (Dasabuvir Sodium Monohydrate); Dasabuvir Sodium: Ombitasvir: Paritaprevir: |
| | Ritonavir; Dasabuvir Sodium: Ombitasvir: Paritaprevir: Ritonavir; Dasatinib |
| | (Dasatinib Monohydrate); Daunorubicin; Daunorubicin (Daunorubicin |
| | Hydrochloride); Dea-Methoxycinnamate; Dead Nettle; Decitabine; Decoquinate; |
| | Deet; Deferasirox; Deferiprone; Deferoxamine Mesylate; Defibrotide; Degarelix |
| | (Degarelix Acetate); Dehydrocholic Acid; Delafloxacin Meglumine; Delavirdine |
| | Mesylate; Delphininum; Delphinium Staphisagria; Delta-9- |
| | Tetrahydrocannabinol (Cannabis Sativa Extract); Dembrexine (Dembrexine |
| | Hydrochloride); Demeclocycline Hydrochloride; Denosumab; Deoxycholic |
| | Acid; Deoxyribonuclease Pancreatic; Deoxyribonucleic Acid; Dequalinium |
| | Chloride; Deracoxib; Dermatophagoides Farinae; Dermatophagoides |
| | Pteronyssinus; Derquantel; Desflurane; Desipramine (Desipramine |
| | Hydrochloride); Desipramine Hydrochloride; Desloratadine; Deslorelin |
| | (Deslorelin Acetate); Desmopressin (Desmopressin Acetate); Desmopressin |
| | Acetate; Desmopressin Acetate (Desmopressin Acetate Trihydrate); |
| | Desmopressin Acetate Trihydrate; Desogestrel; Desonide; Desoximetasone; |
| | Desoxycortone Pivalate; Desvenlafaxine; Desvenlafaxine (Desvenlafaxine |
| | Succinate); Desvenlafaxine Fumarate; Desvenlafaxine Succinate; Detomidine |
| | Hydrochloride; Deutetrabenazine; Dexamethasone; Dexamethasone |
| | (Dexamethasone Acetate); Dexamethasone (Dexamethasone Sodium |
| | Metasulphobenzoate); Dexamethasone (Dexamethasone Sodium Phosphate); |
| | Dexamethasone Acetate; Dexamethasone Phosphate; Dexamethasone Phosphate |
| | (Dexamethasone Sodium Phosphate); Dexamethasone Sodium |
| | Metasulphobenzoate; Dexamethasone Sodium Phosphate; Dexbrompheniramine |
| | Maleate; Dexchlorpheniramine Maleate; Dexlansoprazole; Dexmedetomidine |
| | (Dexmedetomidine Hydrochloride); Dexmedetomidine Hydrochloride; |
| | Dexmethylphenidate Hydrochloride; Dexpanthenol; Dexpanthenol (Calcium D- |
| | Pantothenate); Dexpanthenol (Panthenol); Dexrazoxane; Dextran; Dextran 40; |
| | Dextran 70; Dextroamphetamine Sulfate; Dextromethorphan (Dextromethorphan |
| | Hydrobromide); Dextromethorphan Hydrobromide; Dextromethorphan |
| | Hydrobromide (Dextromethorphan Polistirex); Dextropropoxyphene |
| | Hydrochloride; Dextropropoxyphene Napsylate; Dextrose; Dextrose (Dextrose |
| | Monohydrate); Dextrose Anhydrous; Dextrose Anhydrous (Dextrose |
| | Monohydrate); Dextrothyroxine Sodium; Di-Alkyl Dimethyl Ammonium |
| | Chloride (Mixture); Diadem Spider; Diamorphine Hydrochloride; Diastase; |
| | Diatrizoate Meglumine; Diatrizoate Sodium; Diazepam; Diazoxide; Dibotermin |
| | Alfa; Dibromsalan; Dibucaine; Dibucaine Hydrochloride; Dibunate Sodium; |
| | Dicentra Canadensis; Dichloracetic Acid; Dichlorobenzene; Dichlorobenzyl |
| | Alcohol; Dichlorodifluoromethane; Dichlorophene; Dichlorvos (D.D.V.P.); |
| | Diclazuril; Diclofenac; Diclofenac Diethylamine; Diclofenac Epolamine; |
| | Diclofenac Potassium; Diclofenac Sodium; Diclofenac Sodium: Misoprostol; |
| | Dictamnus Albus; Dicyclomine Hydrochloride; Didanosine; Didecyl Dimethyl |
| | Ammonium Bicarbonate; Didecyl Dimethyl Ammonium Carbonate; Didecyl |
| | Dimethyl Ammonium Chloride; Dienestrol; Dienogest; Diethanolamine |
| | Fusidate; Diethylamine Salicylate; Diethylcarbamazine Citrate; Diethylpropion |
| | Hydrochloride; Diethylstilbestrol; Diethylstilbestrol Diphosphate Sodium; |
| | Diflorasone Diacetate; Difloxacin (Difloxacin Hydrochloride); Diflucortolone |
| | Valerate; Diflunisal; Difluoro Silane; Difluprednate; Digitalis; Digitoxin; |
| | Digoxin; Digoxin Immune Fab (Ovine); Dihydroergotamine Mesylate; |
| | Dihydrostreptomycin (Dihydrostreptomycin Sulfate); Dihydrostreptomycin |
| | Sulfate; Dihydrotachysterol; Diiodohydroxyquin; Dill; Diltiazem Hydrochloride; |
| | Diluent; Dimenhydrinate; Dimercaprol; Dimercaptosuccinic Acid; Dimethicone; |
| | Dimethoxane; Dimethyl Fumarate; Dimethyl Glutarate; Dimethyl Sebacate; |
| | Dimethyl Sulfoxide; Dimethylbenzyl Ammonium Chloride; Dimetridazole; |
| | Dinitolmide; Dinoprost (Dinoprost Tromethamine); Dinoprostone; Dinutuximab; |
| | Dioctyl Dimethyl Ammonium Chloride; Dioscorea Opposita; Dioscorea Villosa; |
| | Dioxybenzone; Diphemanil Methylsulfate; Diphenhydramine; Diphenhydramine |
| | Citrate; Diphenhydramine Hydrochloride; Diphenoxylate Hydrochloride; |
| | Diphenylguanidine; Diphenylpyraline Hydrochloride; Diphtheria Antitoxin; |
| | Diphtheria Toxoid; Diphtheria Toxoid Adsorbed; Diphtheria Toxoid Antigen; |
| | Dipivefrin; Dipivefrin Hydrochloride; Diprenorphine Hydrochloride; |
| | Dipyridamole; Dipyrone; Dipyrone Monohydrate; Dirca Palustris; Dirlotapide; |
| | Disodium Dodecyl Diphenyloxide Disulfonate; Disodium Edetate; Disodium |
| | Lauroamphodiacetate Conc.; Disodium Oxidronate; Disofenin; Disopyramide; |
| | Disopyramide (Disopyramide Phosphate); Disulfiram; Dithiobis |
| | (Benzothiazole); Dithranol; Divalproex Sodium; Dl-Alpha Tocopheryl Acetate; |
| | Dl-Alpha Tocopheryl Acetate (Vitamin E); Dl-Alpha Tocopheryl Acid |
| | Succinate; Dl-Isoleucine; Dl-Lysine Hydrochloride; Dl-Methionine; Dl- |
| | Tryptophane; Dl-Valine; Dobutamine (Dobutamine Hydrochloride); Docetaxel; |
| | Dock; Docosahexaenoic Acid; Docosahexaenoic Acid (Fish Oil); Docosanol; |
| | Docusate Calcium; Docusate Sodium; Dodecyl Dimethyl Benzyl Ammonium |
| | Chloride; Dodecylisoquinolinium Bromide; Dodicin; Dodicin Hydrochloride; |
| | Dofetilide; Dog Hair; Dog Rose; Dog's Mercury; Dolasetron Mesylate; Dolichos |
| | Pruriens; Dolichovespula Arenaria Venom Protein Extract; Dolichovespula |
| | Maculata Venom Protein Extract; Dolutegravir (Dolutegravir Sodium); |
| | Domiphen Bromide; Domperidone (Domperidone Maleate); Donepezil |
| | Hydrochloride; Donepezil Hydrochloride (Donepezil Hydrochloride |
| | Monohydrate); Dopamine Hydrochloride; Doramectin; Doravirine; Doripenem |
| | (Doripenem Monohydrate); Dornase Alfa; Doryphora Decemlineata; |
| | Dorzolamide (Dorzolamide Hydrochloride); Doxacurium (Doxacurium |
| | Chloride); Doxapram Hydrochloride; Doxazosin (Doxazosin Mesylate); Doxepin |
| | (Doxepin Hydrochloride); Doxepin Hydrochloride; Doxercalciferol; |
| | Doxorubicin Hydrochloride; Doxycycline; Doxycycline (Doxycycline Hyclate); |
| | Doxycycline (Doxycycline Monohydrate); Doxycycline Hyclate; Doxylamine |
| | Succinate; Dried Yeast; Drometrizole Trisiloxane; Dronabinol; Dronedarone |
| | (Dronedarone Hydrochloride); Dronedarone Hydrochloride; Droperidol; Drosera |
| | Rotundifolia; Drospirenone; Drotrecogin Alfa (Activated); Duboisia |
| | Myoporoides; Dulaglutide; Dulcamara; Duloxetine (Duloxetine Hydrochloride); |
| | Duloxetine Hydrochloride; Dulse; Dupilumab; Durvalumab; Dust Concentrate; |
| | Dutasteride; Dwarf Mallow; Dwarf Pine; Dyclonine Hydrochloride; Dyphylline |
| E | Ecallantide; Echinacea; Echinacea (Echinacea Angustifolia; Echinacea |
| | (Echinacea Angustifolia); Echinacea (Echinacea Pallida); Echinacea (Echinacea |
| | Purpurea; Echinacea (Echinacea Purpurea); Echinacea Angustifolia; Echinacea |
| | Angustifolia (Echinacea); Echinacea Purpurea; Echinacea Purpurea (Echinacea); |
| | Echinacea Purpurea Aerial Parts; Echinacea Purpurea Roots; Echothiophate |
| | Iodide; Econazole Nitrate; Eculizumab; Edaravone; Edetate Sodium; Edetic |
| | Acid; Edoxaban (Edoxaban Tosylate); Edoxaban Tosylate; Edoxudine; |
| | Edrophonium Chloride; Efalizumab; Efavirenz; Efinaconazole; Eflomithine |
| | Hydrochloride; Egg Phospholipids; Eicosapentaenoic Acid; Eicosapentaenoic |
| | Acid (Fish Oil); Elaeis Guineensis; Elagolix (Elagolix Sodium); Elaterium; |
| | Elbasvir; Eletriptan (Eletriptan Hydrobromide); Eliglustat (Eliglustat Tartrate); |
| | Eliglustat Tartrate; Elm Bark; Elosulfase Alfa; Elotuzumab; Eltrombopag |
| | (Eltrombopag Olamine); Eltrombopag Olamine; Eluxadoline; Elvitegravir; |
| | Emamectin Benzoate; Embutramide; Emedastine (Emedastine Difumarate); |
| | Emetine Hydrochloride; Emicizumab; Emodepside; Empagliflozin; |
| | Empagliflozin: Linagliptin; Empagliflozin: Metformin Hydrochloride; |
| | Emtricitabine; Emtricitabine: Tenofovir Disoproxil Fumarate; Enalapril Maleate; |
| | Enalapril Sodium; Enalapril Sodium (Enalapril Maleate); Enalaprilat; Enasidenib |
| | (Enasidenib Mesylate); Enasidenib Mesylate; Encorafenib; Endive; Enflurane; |
| | Enfuvirtide; Enilconazole; Enoxaparin Sodium; Enrofloxacin; Ensulizole; |
| | Entacapone; Entecavir; Entecavir (Entecavir Monohydrate); Entsufon Sodium; |
| | Enzacamene; Enzalutamide; Ephedra; Ephedra (Ma Huang); Ephedrine; |
| | Ephedrine (Ephedra); Ephedrine As Resin Complex; Ephedrine Hydrochloride; |
| | Ephedrine Racemic (Ephedra; Ephedrine Racemic (Ephedra Sinica); Ephedrine |
| | Racemic (Ephedra); Ephedrine Racemic (Ephedrine Hydrochloride); Ephedrine |
| | Racemic (Ma Huang); Ephedrine Sulfate; Epigaea Repens; Epilobium Palustre; |
| | Epinephrine; Epinephrine (Epinephrine Bitartrate); Epinephrine (Epinephrine |
| | Hydrochloride); Epinephrine (Racemic Epinephrine Hydrochloride); |
| | Epinephrine Bitartrate; Epinephrine Hydrochloride; Epinephrine Racemic; |
| | Epinephrine Racemic (Racemic Epinephrine Hydrochloride); Epiphegus |
| | Virginiana; Epirubicin Hydrochloride; Epithelial Tissue; Eplerenone; Epoetin |
| | Alfa; Epoetin Alfa-Epbx; Epoprostenol (Epoprostenol Sodium); Epoxy Resin; |
| | Eprinomectin; Eprofloxacin; Eprosartan (Eprosartan Mesylate); Epsilon |
| | Aminocaproic Acid; Epsiprantel; Eptacog Alfa (Activated); Eptifibatide; |
| | Equisetum Arvense; Eranthis Hyemalis; Erechtites Hieracifolia; Erenumab; |
| | Ergoloid Mesylates; Ergonovine Maleate; Ergot; Ergotamine (Ergotamine |
| | Tartrate); Ergotamine Tartrate; Eribulin Mesylate; Erica; Erigeron Canadensis L.; |
| | Eriobotrya Japonica; Eriodictyon; Erlotinib (Erlotinib Hydrochloride); Erodium; |
| | Ertapenem (Ertapenem Sodium); Ertugliflozin; Ertugliflozin: Metformin |
| | Hydrochloride; Ertugliflozin: Sitagliptin Phosphate; Erythromycin; |
| | Erythromycin (Erythromycin Estolate); Erythromycin (Erythromycin |
| | Ethylsuccinate); Erythromycin (Erythromycin Glucoheptonate); Erythromycin |
| | (Erythromycin Lactobionate); Erythromycin (Erythromycin Phosphate); |
| | Erythromycin (Erythromycin Stearate); Erythromycin Glucoheptonate; |
| | Erythromycin Stearate; Erythromycin Thiocyanate; Erythrosine; Eschscholtzia |
| | Californica; Escitalopram; Escitalopram (Escitalopram Oxalate); Escitalopram |
| | Oxalate; Esculin; Esketamine Hydrochloride; Eslicarbazepine Acetate; Esmolol |
| | Hydrochloride; Esomeprazole (Esomeprazole Magnesium Dihydrate); |
| | Esomeprazole (Esomeprazole Magnesium Trihydrate); Esomeprazole |
| | (Esomeprazole Magnesium); Esomeprazole Magnesium; Esomeprazole |
| | Magnesium: Naproxen; Esomeprazole Strontium; Estazolam; Esterified |
| | Estrogens; Estradiol; Estradiol (Estradiol Hemihydrate); Estradiol Benzoate; |
| | Estradiol Cypionate; Estradiol Dienanthate; Estradiol Enanthate; Estradiol |
| | Valerate; Estramustine Phosphate Sodium; Estrone; Estropipate; Eszopiclone; |
| | Etafedrin Hydrochloride; Etanercept; Etanercept-Ykro; Ethacridine Lactate; |
| | Ethacrynic Acid; Ethacrynic Acid (Ethacrynate Sodium); Ethambutol |
| | Hydrochloride; Ethanolamine Oleate; Ethchlorvynol; Ethinyl Estradiol; |
| | Ethisterone; Ethoheptazine Citrate; Ethopropazine (Ethopropazine |
| | Hydrochloride); Ethosuximide; Ethotoin; Ethoxyquin; Ethyl Acetate; Ethyl |
| | Cellulose; Ethyl Chloride; Ethyl Ether; Ethyl Paraben; Ethyl Salicylate; Ethyl |
| | Sulfide; Ethylene Oxide; Ethylenediamine Dihydrochloride; Ethylenediamine |
| | Dihydroiodide; Ethynodiol Diacetate; Etidronate Disodium; Etodolac; |
| | Etonogestrel; Etoposide; Etorphine Hydrochloride; Etravirine; Eucalyptol; |
| | Eucalyptus; Eucalyptus Gum; Eucalyptus Oil; Eucommiae Ulmoidis; Eugenia |
| | Jambolana; Eugenia Jambosa; Eugenol; Euonymus Atropurpureus; Euonymus |
| | Europaeus; Eupatorium Aromaticum; Eupatorium Cannabinum; Eupatorium |
| | Perfoliatum; Eupatorium Purpureum; Euphorbia; Euphorbia Amygdaloides; |
| | Euphorbia Corollata; Euphorbia Cyparissias; Euphorbia Hypericifolia; |
| | Euphorbia Lathyris; Euphorbia Pilulifera; Euphorbium Officinarum; Euphrasia |
| | Officinalis; Eupion; Evening Primrose Oil; Everolimus; Evolocumab; |
| | Exametazime; Exemestane; Exenatide; Exenatide Synthetic; Exocarpium Citri; |
| | Eye Tissue Extract; Ezetimibe; Ezogabine |
| F | Factor Ix Complex Human; Factor Xiii; Fagus Sylvatica; Famciclovir; |
| | Famotidine; Famotidine: Ibuprofen; Fampridine; Fatty Acids; Fatty Acids |
| | (Borage Oil); Fatty Acids Unsaturated; Feathers; Febantel; Febuxostat; |
| | Febuxostat (Febuxostat Hemihydrate); Fel Tauri; Felbamate; Felodipine; |
| | Fenbendazole; Fenfluramine Hydrochloride; Fennel; Fennel Oil; Fenofibrate; |
| | Fenoprofen (Fenoprofen Calcium); Fenoprofen Calcium; Fenoterol |
| | Hydrobromide; Fenprostalene; Fentanyl; Fentanyl (Fentanyl Citrate); Fentanyl |
| | Citrate; Fenugreek; Ferric Ammonium Citrate; Ferric Chloride; Ferric Citrate; |
| | Ferric Ferrocyanide; Ferric Hydroxide; Ferric Hypophosphite; Ferric Nitrate; |
| | Ferric Phosphate; Ferric Pyrophosphate; Ferric Sulfate; Ferrous Bromide; |
| | Ferrous Carbonate Saccharated; Ferrous Fumarate; Ferrous Gluconate; Ferrous |
| | Iodide; Ferrous Lactate; Ferrous Phosphate; Ferrous Sulfate; Ferrous Tartrate; |
| | Ferrum Aceticum; Ferrum Phosphoricum; Ferrum Picricum; Ferrum Sidereum; |
| | Ferula Glauca; Fesoterodine Fumarate; Feverfew; Feverfew (Tanacetum |
| | Parthenium); Fexofenadine Hydrochloride; Fiber; Fibrinogen (Human); |
| | Fibrinolysin; Ficus Religiosa; Fidaxomicin; Field Mint Leaf; Fig; Figwort; |
| | Filamentous Haemagglutinin; Filamentous Haemagglutinin Adsorbed; |
| | Filgrastim; Filipendula Ulmaria; Fimbriae; Fimbriae Types 2 And 3 (Fim); |
| | Finafloxacin; Finasteride; Fingolimod (Fingolimod Hydrochloride); Fingolimod |
| | Hydrochloride; Fir; Firocoxib; Fish Liver Oil; Fish Oil; Flavoxate Hydrochloride; |
| | Flecainide Acetate; Flibanserin; Floctafenine; Florbetaben (18f); Florfenicol; |
| | Flos Lonicerae; Floxacillin (Floxacillin Sodium); Fluconazole; Fludarabine |
| | Phosphate; Fludarabine Phosphate (Fludarabine Phosphate Sodium); |
| | Fludeoxyglucose 18f; Fludrocortisone 21-Acetate; Flumazenil; Flumethasone; |
| | Flumethasone Pivalate; Flunarizine (Flunarizine Hydrochloride); Flunarizine |
| | Hydrochloride; Flunisolide; Flunixin (Flunixin Meglumine); Fluocinolone |
| | Acetonide; Fluocinonide; Fluorescein (Fluorescein Sodium); Fluorescein |
| | Sodium; Fluoride (Potassium Fluoride); Fluoride (Sodium Fluoride; Fluoride |
| | (Sodium Fluoride); Fluorine; Fluorine (Calcium Fluoride); Fluorine (Potassium |
| | Fluoride); Fluorine (Sodium Fluoride); Fluorometholone; Fluorometholone |
| | Acetate; Fluorouracil; Fluorouracil (Fluorouracil Sodium); Fluoxetine |
| | (Fluoxetine Hydrochloride); Fluoxetine Hydrochloride; Fluoxetine |
| | Hydrochloride: Olanzapine; Fluoxymesterone; Flupentixol (Flupentixol |
| | Dihydrochloride); Flupentixol Decanoate; Flupentixol Dihydrochloride; |
| | Fluphenazine Decanoate; Fluphenazine Enanthate; Fluphenazine Hydrochloride; |
| | Fluralaner; Flurandrenolide; Flurazepam Hydrochloride; Flurbiprofen; |
| | Flurbiprofen Sodium; Fluspirilene; Flutamide; Fluticasone Furoate; Fluticasone |
| | Furoate: Umeclidinium Bromide: Vilanterol Trifenatate; Fluticasone Propionate; |
| | Fluvastatin (Fluvastatin Sodium); Fluvoxamine Maleate; Foeniculum Vulgaris; |
| | Folic Acid; Folic Acid (Cyanocobalamin); Folic Acid (Liver); Folic Acid |
| | (Sodium Folate); Folic Acid (Yeast); Follicle Stimulating Hormone (Fsh); |
| | Follicle Stimulating Hormone (Fsh)-Porcine; Follitropin Alfa; Follitropin Beta; |
| | Follitropin Delta; Fomepizole; Fondaparinux Sodium; Formaldehyde; |
| | Formestane; Formic Acid; Formica; Formica Rufa; Formocresol; Formoterol |
| | Fumarate; Formoterol Fumarate Dihydrate; Formoterol Fumarate: |
| | Glycopyrrolate; Forsythia Suspensa; Fosamprenavir (Fosamprenavir Calcium); |
| | Fosaprepitant (Fosaprepitant Dimeglumine); Fosfomycin (Fosfomycin Sodium); |
| | Fosfomycin (Fosfomycin Tromethamine); Fosinopril Sodium; Fosphenytoin |
| | Sodium; Framycetin (Framycetin Sulfate); Framycetin Sulfate; Franciscea |
| | Uniflora Homeo; Fraxinus Americana; Fraxinus Excelsior; Fringe Tree; |
| | Fritillaria Cirrhosa; Frovatriptan (Frovatriptan Succinate Monohydrate); |
| | Frovatriptan (Frovatriptan Succinate); Fructus Gardeniae Jasminoidis; Fructus |
| | Oryzae Germinatus; Fucus Vesiculosus; Fulvestrant; Fumagillin; Fumagillin |
| | (Fumagillin Dicyclohexylamine); Fumagillin Dicyclohexylamine; Fumaria |
| | Officinalis; Furazolidone; Furfural; Furosemide; Fusidate Sodium; Fusidic Acid |
| G | Gabapentin; Gabapentin Enacarbil; Gadobenate Dimeglumine; Gadobutrol; |
| | Gadodiamide; Gadofosveset Trisodium; Gadopentetate Dimeglumine; |
| | Gadoterate Meglumine; Gadoteridol; Gadoversetamide; Gadoxetate Disodium; |
| | Galactose; Galangal Root; Galantamine (Galantamine Hydrobromide); |
| | Galanthus Nivalis; Galega Officinalis; Gallamine Triethiodide; Gallbladder; |
| | Gallic Acid; Gallium Citrate Ga 67; Galphimia Glauca; Galsulfase; Gambir; |
| | Gamboge; Gamithromycin; Gamma-Linolenic Acid; Gamma-Linolenic Acid |
| | (Black Currant); Gamma-Linolenic Acid (Borage Oil; Gamma-Linolenic Acid |
| | (Borage Oil); Gamma-Linolenic Acid (Borago Officinalis); Gamma-Linolenic |
| | Acid (Evening Primrose Oil; Gamma-Linolenic Acid (Evening Primrose Oil); |
| | Ganciclovir; Ganciclovir (Ganciclovir Sodium); Ganciclovir Sodium; Ganirelix |
| | (Ganirelix Acetate); Ganoderma Lucidum; Garlic; Gastrodiae Rhizoma; |
| | Gatifloxacin; Gatifloxacin (Gatifloxacin Hemihydrate); Gaultheria Procumbens; |
| | Gefitinib; Gelatin; Gelsemium Sempervirens; Gemcitabine (Gemcitabine |
| | Hydrochloride); Gemfibrozil; Gemifloxacin (Gemifloxacin Mesylate); |
| | Gemifloxacin Mesylate; Genista; Gentamicin (Gentamicin Sulfate); Gentamicin |
| | (Gentamycin Sulfate); Gentamicin Sulfate; Gentian Violet; Gentiana Cruciata; |
| | Gentiana Lutea; Gentiana Quinqueflora; Gentianella Amarella; Geraniol; |
| | Geranium Maculatum; Geranium Robertianum; German Chamomile; German |
| | Chamomile (Matricaria Chamomilla); Gilsonite; Gilteritinib Fumarate; Ginger; |
| | Ginger (Zingiber Officinale); Ginkgo Biloba; Ginseng; Glandular Extract |
| | Homeopathic; Glatiramer Acetate; Glecaprevir; Gleptoferron (Iron Dextran |
| | Complex); Gliclazide; Glimepiride; Glimepiride: Pioglitazone Hydrochloride; |
| | Glimepiride: Rosiglitazone Maleate; Glucagon (Glucagon Hydrochloride); |
| | Glucagon (Recombinant Dna Origin); Glucagon (Recombinant Dna Origin) |
| | (Glucagon Hydrochloride); Gluconic Acid; Glutamic Acid; Glutamic Acid |
| | Hydrochloride; Glutamine; Glutaral; Glutathione; Glutathione Disulfide; |
| | Glyburide; Glycerine; Glycerol Monolaurate; Glycerol Phenylbutyrate; |
| | Glycerophosphate Potassium; Glyceryl Mono-Oleate; Glycine; Glycol |
| | Salicylate; Glycolic Acid; Glycopyrrolate; Glycopyrronium (Glycopyrronium |
| | Bromide); Glycosaminoglycan Polysulfate; Glycyrrhiza Glabra; Glycyrrhiza |
| | Uralensis; Glycyrrhizin; Glyoxal; Gnaphalium Leontopodium; Gnaphalium |
| | Polycephalum; Goats Beard's Herb; Gold; Gold Sodium Thiomalate; Gold |
| | Trichloride; Goldenrod; Golimumab; Gonadorelin (Gonadorelin Acetate); |
| | Gonadorelin (Gonadorelin Decapeptide (Gnrh)); Gonadorelin (Gonadorelin |
| | Hydrochloride); Gonadorelin Acetate; Gonadorelin Diacetate Tetrahydrate; |
| | Gonadorelin Hydrochloride; Gonadotrophin Pituitary; Gonadotrophin Serum |
| | (Pmsg); Gonadotropin-Releasing Factor Analog-Diphtheria Toxoid Conjugate; |
| | Goose Grass; Goserelin (Goserelin Acetate); Gramicidin; Granisetron; |
| | Granisetron (Granisetron Hydrochloride); Grape; Graphite; Grapiprant; Grass |
| | Pollen Allergen Extract; Gratiola Officinalis; Grazoprevir; Grepafloxacin |
| | (Grepafloxacin Hydrochloride); Griseofulvin; Ground Ivy; Groundsel; Guaiac; |
| | Guaiacol; Guaiacol Carbonate; Guaiacol Oleate; Guaiacum; Guaifenesin; |
| | Guaifenesin: Hydrocodone Bitartrate; Guanethidine Sulfate; Guanfacine |
| | (Guanfacine Hydrochloride); Guarana; Guinea Pepper; Gumweed; Gunpowder; |
| | Guselkumab; Gymnocladus Canadensis |
| H | Haemagglutinin From A/California/7/2009 (H1n1)V-Like Strain (X-179a); |
| | Haemagglutinin-Strain A(H1n1); Haemagglutinin-Strain A(H3n2); |
| | Haemagglutinin-Strain A(H5n1); Haemagglutinin-Strain B; Haemagglutinin- |
| | Strain B (Victoria); Haemagglutinin-Strain B (Yamagata); Haemophilus |
| | Influenzae Type B-Prp; Haemophilus Influenzae Type B-Prp And Tetanus |
| | Toxoid Conjugate (Prp-T); Haemophilus Type B Oligosaccharide; Hahnemann's |
| | Causticum; Halcinonide; Halethazole Tartrate; Halloysite; Halobetasol |
| | Propionate; Halofantrine Hydrochloride; Halofuginone (Halofuginone |
| | Hydrobromide); Halofuginone (Halofuginone Lactate); Halofuginone |
| | Hydrobromide; Haloperidol; Haloperidol (Haloperidol Decanoate); Haloperidol |
| | Decanoate; Halothane; Hamamelis Virginiana; Hamamelis Virginiana Water; |
| | Haptens; Hard Fat; Harpagophytum Procumbens; Hawthorn; Hazel Tree; Hecla |
| | Lava; Helianthemum Nummularium; Heliotropium Peruvianum; Helium; |
| | Helleborus; Helleborus Niger; Heloderma; Helonias; Hemicellulase; Hemin; |
| | Hemlock Spruce; Hemoglobin (Crystals Etc.); Hemophilus Influenzae; Heparin |
| | (Heparin Sodium); Heparin Calcium; Heparin Sodium; Hepatitis A Vaccine; |
| | Hepatitis B Immunoglobulin (Human); Hepatitis B Surface Antigen |
| | (Recombinant); Heracleum Sphondylium; Herba Menthae Haplocalycis; Herbals |
| | Homeopathic; Herniaria; Hesperidin; Hetastarch; Hexachlorophene; Hexamidine |
| | Isethionate; Hexaminolevulinate Hydrochloride; Hexetidine; Hexylresorcinol; |
| | Hieracium Pilosella; Hippuric Acid; Hirudin; Histamine; Histamine (Histamine |
| | Dihydrochloride); Histamine Dihydrochloride; Histamine Phosphate; Histidine; |
| | Histidine Monohydrochloride; Histrelin Acetate; Hoang Nan; Hogfennel Root; |
| | Hoitzia Coccinea; Holarrhena Antidysenterica; Homarus; Homatropine |
| | Hydrobromide; Homatropine Methylbromide; Homatropine Methylbromide: |
| | Hydrocodone Bitartrate; Homosalate; Honey; Honey Bee Venom Protein; Hops; |
| | Hops (Humulus Lupulus); Horehound; Horse Hair; Horse Radish; Hottonia |
| | Palustris; Human Immunoglobulins (Anti-D (Rh) Immunoglobulin (Human)); |
| | Human Immunoglobulins (Tetanus Immunoglobulin (Human)); Human Plasma; |
| | Human Plasma Proteins; Human-Bovine Rotavirus Reassortant Type G1; |
| | Human-Bovine Rotavirus Reassortant Type G2; Human-Bovine Rotavirus |
| | Reassortant Type G3; Human-Bovine Rotavirus Reassortant Type G4; Human- |
| | Bovine Rotavirus Reassortant Type P1; Hura Brasiliensis; Hura Crepitans; |
| | Hyaluronate Sodium; Hyaluronidase; Hydralazine Hydrochloride; Hydrangea |
| | Arborescens; Hydrastine Hydrochloride; Hydrastis Canadensis; Hydrochloric |
| | Acid; Hydrochlorothiazide; Hydrocodone; Hydrocodone Bitartrate; |
| | Hydrocodone Bitartrate: Ibuprofen; Hydrocodone Bitartrate: Pseudoephedrine |
| | Hydrochloride; Hydrocortisone; Hydrocortisone (Adrenal Cortical Extract); |
| | Hydrocortisone (Hydrocortisone Acetate); Hydrocortisone (Hydrocortisone |
| | Sodium Succinate); Hydrocortisone Aceponate; Hydrocortisone Acetate; |
| | Hydrocortisone Sodium Succinate; Hydrocortisone Valerate; Hydrocotyle; |
| | Hydrocyanic Acid; Hydrogen Fluoride; Hydrogen Peroxide; Hydrogen Sulfide; |
| | Hydromorphone Hydrochloride; Hydrophyllum Virginianum; Hydroquinone; |
| | Hydroxocobalamin; Hydroxychloroquine Sulfate; Hydroxycitronellal; |
| | Hydroxyethyl Starch 130/0.4; Hydroxyethylcellulose; Hydroxyurea; |
| | Hydroxyzine Hydrochloride; Hygromycin (Hygromycin B); Hygromycin B; |
| | Hyoscine Butylbromide; Hyoscyamine; Hyoscyamine Hydrobromide; |
| | Hyoscyamine Sulfate; Hyoscyamus Niger; Hypericum Perforatum; |
| | Hypochlorous Acid; Hypothalamus; Hyprolose; Hypromellose; Hyssop; |
| I | Ibandronate Sodium; Ibandronic Acid (Ibandronate Sodium); Iberis Amara; |
| | Ibritumomab Tiuxetan; Ibrutinib; Ibuprofen; Ibuprofen (Ibuprofen); Ibuprofen |
| | (Ibuprofen Sodium Dihydrate); Ibuprofen: Oxycodone Hydrochloride; Ibutilide |
| | Fumarate; Icatibant (Icatibant Acetate); Ichthammol; Icodextrin; Idarubicin |
| | Hydrochloride; Idarucizumab; Idebenone; Idelalisib; Idoxuridine; Idursulfase; |
| | Ifosfamide; Ilex Aquifolium; Imatinib (Imatinib Mesylate); Imidacloprid; |
| | Imidapril Hydrochloride; Imiglucerase; Imipenem; Imipramine Hydrochloride; |
| | Imipramine Pamoate; Imiquimod; Immunoglobulin (Human); Immunoglobulin |
| | G (Human); Impatiens Glandulifera; Inact. Japanese Encephal Virus(Attenuat. |
| | Strain Sa14-14-2 Produced In Vero Cells); Inactivated Poliomyelitis Vaccine |
| | (D.C.O.) Type 1 Mahoney; Inactivated Poliomyelitis Vaccine (D.C.O.) Type 2 |
| | Mef1; Inactivated Poliomyelitis Vaccine (D.C.O.) Type 3 Saukett; Inactivated |
| | Poliomyelitis Vaccine (V.C.O.) Type 1 Mahoney; Inactivated Poliomyelitis |
| | Vaccine (V.C.O.) Type 2 Mef1; Inactivated Poliomyelitis Vaccine (V.C.O.) Type |
| | 3 Saukett; Inactivated Poliovirus Type I; Inactivated Poliovirus Type Ii; |
| | Inactivated Poliovirus Type Iii; Inamrinone (Amrinone Lactate); |
| | Incobotulinumtoxina; Indacaterol (Indacaterol Maleate); Indacaterol Maleate; |
| | Indapamide; Indigo; Indigo Carmine; Indinavir (Indinavir Sulfate); Indium In |
| | 111 Chloride; Indium Metallicum; Indium Oxine; Indocyanine Green; Indolum; |
| | Indomethacin; Indomethacin (Sodium Indomethacin); Infliximab; Infliximab- |
| | Abda; Infliximab-Dyyb; Infliximab-Qbtx; Influenza Virus Type A (H1n1); |
| | Influenza Virus Type A (H3n2); Influenza Virus Type B; Influenza Virus Type |
| | B (Victoria); Influenza Virus Type B (Yamagata); Influenza Virus Vaccine; |
| | Ingenol Mebutate; Inosine Pranobex; Inositol; Inositol (Calcium D- |
| | Pantothenate); Inositol (Calcium Magnesium Phytate); Inositol (Calcium |
| | Phosphate (Dibasic); Inositol (Choline Citrate); Inositol (Chromium Hvp |
| | Chelate); Inositol (Inositol Hexanicotinate); Inositol (Inositol Niacinate); Inositol |
| | (Inositol Phosphatides); Inositol (Potassium Chloride); Inositol (Vegetables; |
| | Inositol (Yeast); Inotersen (Inotersen Sodium); Inotersen Sodium; Inotuzumab |
| | Ozogamicin; Insulin; Insulin (Human); Insulin (Pork); Insulin Aspart; Insulin |
| | Aspart Protamine; Insulin Biosynthetic Human Br; Insulin Degludec; Insulin |
| | Degludec: Liraglutide; Insulin Detemir; Insulin Glargine; Insulin Glargine: |
| | Lixisenatide; Insulin Glulisine (Recombinant Dna Origin); Insulin Injection |
| | Human Biosynthetic; Insulin Injection Human Semi-Synthetic; Insulin Injection |
| | Pork; Insulin Isophane (Nph); Insulin Isophane Human Biosynthetic; Insulin |
| | Isophane Injection Pork; Insulin Isophane Nph Pork; Insulin Lispro; Insulin |
| | Lispro Protamine Suspension; Insulin Nph Human Dna Origin; Insulin |
| | Recombinant Human; Insulin Semi Synthetic Human; Insulin Sulphated Beef; |
| | Insulin Zinc; Insulin Zinc Human Biosynthetic; Insulin Zinc Pork; Insulin Zinc |
| | Sus-Medium Human Biosynthetic; Insulin Zinc Sus-Prolonged Human |
| | Biosynthetic; Interferon Alfa-2b; Interferon Alfacon-1; Interferon Alpha-2a; |
| | Interferon Alpha-N1 (Ins); Interferon Beta-1a; Interferon Beta-1b; Intrinsic |
| | Factor Conc; Inula Helenium; Invert Sugar; Invertase; Iobenguane 123 I; |
| | Iobenguane 131i; Iodide (Potassium Iodide); Iodide (Sodium Iodide); Iodinated |
| | Casein; Iodine; Iodine (Alkoxypolyethoxyethanol Iodized Complex); Iodine |
| | (Alkyl Alcohol Ethoxylated Iodine Complex); Iodine (Alkyl Poly Ethoxy Ethanol - |
| | Iodine Complex); Iodine (Calcium Iodate); Iodine (Ethiodized Oil); Iodine |
| | (Ethylenediamine Dihydroiodide); Iodine (Hydriodic Acid - Iodine Complex); |
| | Iodine (Iodinated Casein); Iodine (Iodinated Protein Hydrolysate); Iodine (Iodine |
| | Hvp Chelate); Iodine (Iodine); Iodine (Iodophor); Iodine (Iohexol); Iodine |
| | (Iopamidol); Iodine (Iotrolan); Iodine (Ioversol); Iodine (Kelp; Iodine (Kelp); |
| | Iodine (Nonyl Phenoxy Polyoxyethylene Ethanol-Iodine Complex); Iodine |
| | (Nonyl Phenoxypoly Ethoxy Ethanol-Iodine); Iodine (Nonyl |
| | Phenoxypolyethoxyethanol-Iodine Complex); Iodine |
| | (Nonylphenoxypoly(Ethyleneoxy)Ethanol Iodine Complex); Iodine |
| | (Nonylphenyl-Omega-Hydroxypoly Oxyethylene - Iodine Complex); Iodine |
| | (Poloxamer Iodine Complex); Iodine (Polyalkylene Glycol Ether Iodophor - |
| | Complex); Iodine (Polyalkylene Glycol Ether Iodophor); Iodine (Polyethoxy |
| | Polypropoxy Polyethoxy Ethanol - Iodine Complex); Iodine (Polyoxyethylene- |
| | Polyoxypropylene Block Copolymer-Iodine Complex); Iodine |
| | (Polyoxypropylene-Polyoxyethylene Block Copolymer-Iodine Complex); Iodine |
| | (Potassium Iodide; Iodine (Potassium Iodide); Iodine (Povidone-Iodine); Iodine |
| | (Sodium Iodide); Iodine Ethoxylate; Iodixanol; Iodoform; Iodophor; Ioflupane |
| | (123i); Iohexol; Iopamidol; Iopanoic Acid; Iopromide; Iopydol; Iopydone; |
| | Iothalamate Meglumine; Iothalamate Sodium; Ioversol; Ioxaglate Meglumine; |
| | Ioxaglate Sodium; Ipecac; Ipilimumab; Ipodate Sodium; Ipomoea Stans; |
| | Ipratropium Bromide; Ipratropium Bromide (Ipratropium Bromide |
| | Monohydrate); Irbesartan; Iridium Metallicum; Irinotecan Hydrochloride; |
| | Irinotecan Hydrochloride (Irinotecan Hydrochloride Trihydrate); Irinotecan |
| | Hydrochloride (Irinotecan Sucrose Octasulfate); Iris Fiorentina; Iris |
| | Foetidissima; Iris Germanica; Iris Tenax; Iris Versicolor; Iron; Iron (Ferric |
| | Ammonium Citrate); Iron (Ferric Citrate); Iron (Ferric Glycerophosphate); Iron |
| | (Ferric Hydroxide Dextran Complex); Iron (Ferric Hydroxide Sucrose Complex); |
| | Iron (Ferronyl); Iron (Ferrous Ascorbate); Iron (Ferrous Carbonate Saccharated); |
| | Iron (Ferrous Citrate; Iron (Ferrous Fumarate; Iron (Ferrous Fumarate); Iron |
| | (Ferrous Gluconate; Iron (Ferrous Gluconate); Iron (Ferrous Lactate); Iron |
| | (Ferrous Phosphate); Iron (Ferrous Succinate); Iron (Ferrous Sulfate); Iron |
| | (Ferumoxytol); Iron (Gleptoferron); Iron (Iron Amino Acid Chelate); Iron (Iron |
| | Citrate; Iron (Iron Citrate); Iron (Iron Dextran Complex); Iron (Iron Dextran); |
| | Iron (Iron Glutarate); Iron (Iron Glycerophosphate); Iron (Iron Hvp Chelate; Iron |
| | (Iron Hvp Chelate); Iron (Iron Isomaltoside 1000); Iron (Iron Malate); Iron (Iron |
| | Organic Complex); Iron (Iron Oxide); Iron (Iron Peptonized); Iron (Iron |
| | Proteinate; Iron (Iron Proteinate); Iron (Iron Reduced); Iron (Iron Sucrose); Iron |
| | (Polysaccharide-Iron Complex); Iron (Sodium Ferric Gluconate Complex In |
| | Sucrose Injection); Iron (Sucroferric Oxyhydroxide); Iron Amino Acid Chelate; |
| | Iron Dextran Complex; Iron Hvp Chelate; Iron Hvp Chelate (Ferrous Gluconate); |
| | Iron Oxide; Iron Peptonized; Iron Proteinate; Iron Reduced; Iron Sorbitex; Iron |
| | Subsulfate; Isatis Tinctoria; Isavuconazole (Isavuconazonium Sulfate); |
| | Isocarboxazid; Isoeugenol; Isoflupredone Acetate; Isoflurane; Isoleucine; |
| | Isoniazid; Isopropamide (Isopropamide Iodide); Isopropamide Iodide; Isopropyl |
| | Alcohol; Isopropyl Myristate; Isoproterenol Hydrochloride; Isosorbide Dinitrate; |
| | Isosorbide-5-Mononitrate; Isosulfan Blue; Isotretinoin; Italian Rye Grass; |
| | Itraconazole; Ivabradine; Ivabradine (Ivabradine Hydrochloride); Ivabradine |
| | Hydrochloride; Ivacaftor; Ivermectin; Ivermectin B1a; Ivosidenib; Ivy; Ixazomib |
| | (Ixazomib Citrate); Ixekizumab |
| J | Jacaranda Caroba; Jalap; Jalap Resin; Jamaica Dogwood; Japanese Encephalitis |
| | Virus Vaccine; Jatropha Curcas; Jequirity; Jonesia Asoca; Juglans Cinerea; |
| | Juglans Nigra; Juglans Pulv; Juglans Regia; Juniper Berries; Juniper Tar; |
| | Juniperus Virginiana |
| K | Kali Cyanatum; Kali Ferrocyanatum; Kali Picricum; Kali Telluricum; Kalmia |
| | Latifolia; Kaolin; Kava; Kelp; Kerosine; Ketamine (Ketamine Hydrochloride); |
| | Ketanserin (Ketanserin Tartrate); Ketazolam; Ketoconazole; Ketoprofen; |
| | Ketorolac Tromethamine; Ketotifen (Ketotifen Fumarate); Ketotifen Fumarate; |
| | Kidney; Klebsiella Pneumoniae; Knot Grass; Kola; Krameria Triandra |
| L | L-Alanine; L-Arginine; L-Aspartic Acid; L-Cysteine; L-Cysteine (L-Cysteine |
| | Hydrochloride); L-Cysteine Hydrochloride; L-Ephedrine Hydrochloride; L- |
| | Ephedrine Hydrochloride (Ephedra); L-Isoleucine; L-Leucine; L-Leucine |
| | Hydrochloride; L-Lysine; L-Lysine (L-Lysine Acetate); L-Lysine (L-Lysine |
| | Hydrochloride); L-Lysine (Lysine Acetate); L-Lysine Hydrochloride; L- |
| | Menthone; L-Ornithine; L-Ornithine Dihydrochloride; L-Phenylalanine; L- |
| | Phenylalanine Hydrochloride; L-Proline; L-Threonine; L-Tyrosine; L-Valine; |
| | Labetalol Hydrochloride; Laburnum Anagyroides; Lac Caninum; Lac |
| | Defloratum; Lac Felinum; Lac Vaccinum; Lacerta Agilis; Lachesis; Lachnanthes |
| | Tinctoria (Homeo); Lacosamide; Lactase; Lactase (Aspergillus Oryzae); Lactic |
| | Acid; Lactic Acid (Sodium Lactate); Lactin; Lactobacillus Acidophilus; |
| | Lactobacillus Bulgaricus; Lactobacillus Casei; Lactobacillus Rhamnosus; |
| | Lactose; Lactulose; Ladyslipper; Lamb's Quarters; Lamivudine; Lamivudine: |
| | Nevirapine: Zidovudine; Lamotrigine; Lanadelumab; Lanolin; Lanolin - |
| | Anhydrous; Lanreotide (Lanreotide Acetate); Lansoprazole; Lansoprazole: |
| | Naproxen; Lanthanum (Lanthanum Carbonate Hydrate); Lanthanum Carbonate; |
| | Lapatinib (Lapatinib Ditosylate Monohydrate); Lapis Albus; Larch Agaric; Lard; |
| | Larix Decidua; Larkspur; Laronidase; Lasalocid Sodium; Latanoprost; |
| | Latanoprostene Bunod; Lathyrus Sativus; Latrodectus Katipo; Latrodectus |
| | Mactans; Laureth 4; Lavandula Stoechas; Lavender; Lavender Oil; Lead Acetate; |
| | Lead Iodide; Lead Monoxide; Ledipasvir; Ledum Palustre; Leek; Leflunomide; |
| | Lemna Minor; Lemon; Lenalidomide; Lentil; Lenvatinib (Lenvatinib Mesylate); |
| | Lepidium Bonariense; Lepirudin; Lesinurad; Lespedeza Capitata; Letermovir; |
| | Letrozole; Lettuce; Leucine; Leucovorin (Leucovorin Calcium); Leuprolide |
| | Acetate; Levamisole; Levamisole (Levamisole Hydrochloride); Levamisole |
| | (Levamisole Resinate); Levamisole Hydrochloride; Levamisole Hydrochloride |
| | (Levamisole Phosphate); Levamisole Phosphate; Levamisole Resinate; |
| | Levetiracetam; Levobunolol Hydrochloride; Levocabastine (Levocabastine |
| | Hydrochloride); Levocamitine; Levodopa; Levofloxacin; Levofloxacin |
| | (Levofloxacin Hemihydrate); Levomefolate Calcium; Levomenthol; |
| | Levomilnacipran (Levomilnacipran Hydrochloride); Levomilnacipran |
| | Hydrochloride; Levonorgestrel; Levothyroxine Sodium; Liatris Spicata; |
| | Lidocaine; Lidocaine (Lidocaine Hydrochloride Monohydrate); Lidocaine |
| | (Lidocaine Hydrochloride); Lidocaine Hydrochloride; Lidocaine Hydrochloride |
| | (Lidocaine Hydrochloride Monohydrate); Lidocaine Hydrochloride (Povidone- |
| | Iodine); Lifitegrast; Ligustrum Lucidum Fruit; Lilac; Lime Tree Flower; |
| | Limulus; Linaclotide; Linagliptin; Linagliptin: Metformin Hydrochloride; |
| | Linaria Canadensis; Lincomycin; Lincomycin (Lincomycin Hydrochloride); |
| | Lincomycin Hydrochloride; Lindane; Linear Dodecyl Benzene Sulfonic Acid; |
| | Linezolid; Linoleic Acid; Linoleic Acid (Borage Oil); Linoleic Acid (Cod Liver |
| | Oil); Linoleic Acid (Evening Primrose Oil); Linolenic Acid; Linolenic Acid (Cod |
| | Liver Oil); Linum Usitatissimum; Lion's Foot (Paw); Liothyronine (Liothyronine |
| | Sodium); Liothyronine Sodium; Lipase; Lipase (Aspergillus Niger); Lipase |
| | (Rhizopus Niveus); Lipolytic Enzyme; Lipoprotein - Ospa Antigen |
| | Recombinant; Liraglutide; Liraglutide Recombinant; Lisdexamfetamine |
| | Dimesylate; Lisinopril; Lisinopril (Lisinopril Dihydrate); Lithium (Lithium |
| | Citrate); Lithium Carbonate; Lithium Citrate; Lithospermum Officinale; Liver |
| | (Liver Extract); Liver Extract; Liver Soluble Powder; Liverwort; Lixisenatide; |
| | Lobelia Cardinalis; Lobelia Inflata; Lodoxamide (Lodoxamide Tromethamine); |
| | Lolium Temulentum; Lomitapide (Lomitapide Mesylate); Lomitapide Mesylate; |
| | Lomustine; Lonicera Caprifolium; Lonoctocog Alfa; Lopatherum Gracile |
| | Brongniart; Loperamide Hydrochloride; Lophophytum Leandri; Lopinavir; |
| | Lopinavir: Ritonavir; Loratadine; Lorazepam; Lorlatinib; Losartan Potassium; |
| | Loteprednol Etabonate; Lotilaner; Lotus Comiculatus; Lovage; Lovastatin; |
| | Loxapine; Loxapine (Loxapine Hydrochloride); Loxapine (Loxapine Succinate); |
| | Loxapine Succinate; Lubabegron (Lubabegron Fumarate); Lubiprostone; |
| | Lufenuron; Luffa Operculata; Lumacaftor; Lupulus; Lurasidone Hydrochloride; |
| | Luteinizing Hormone; Luteinizing Hormone (Porcine Pituitary Lutropin |
| | Extract); Lutetium (177lu) Oxodotreotide; Lutropin Alfa; Lycopodium; |
| | Lycopodium Clavatum; Lycopus Europaeus; Lycopus Virginicus; Lymphocyte |
| | Immunoglobulin Anti-Thymocyte Globulin (Equine); Lysimachia Nummularia; |
| | Lysine; Lysine (L-Lysine Acetate); Lysine (L-Lysine Hydrochloride); Lysine |
| | (Lysine Acetate); Lysine (Lysine Monohydrochloride); Lysine Acetate |
| M | Macitentan; Macrogol; Maduramicin Ammonium; Mafenide (Mafenide |
| | Acetate); Magaldrate; Magnesium; Magnesium (Dolomite; Magnesium |
| | (Dolomite); Magnesium (Magnesium Amino Acid Chelate); Magnesium |
| | (Magnesium Ascorbate); Magnesium (Magnesium Borogluconate); Magnesium |
| | (Magnesium Carbonate; Magnesium (Magnesium Carbonate); Magnesium |
| | (Magnesium Chloride); Magnesium (Magnesium Citrate; Magnesium |
| | (Magnesium Citrate); Magnesium (Magnesium Fumarate; Magnesium |
| | (Magnesium Glucoheptonate); Magnesium (Magnesium Gluconate); Magnesium |
| | (Magnesium Glycerophosphate); Magnesium (Magnesium Hvp Amino Acid |
| | Chelate); Magnesium (Magnesium Hvp Chelate; Magnesium (Magnesium Hvp |
| | Chelate); Magnesium (Magnesium Hydroxide); Magnesium (Magnesium |
| | Hypophosphite); Magnesium (Magnesium Lactate); Magnesium (Magnesium |
| | Malate); Magnesium (Magnesium Organic Complex); Magnesium (Magnesium |
| | Oxide; Magnesium (Magnesium Oxide); Magnesium (Magnesium Phosphate |
| | Dibasic); Magnesium (Magnesium Proteinate; Magnesium (Magnesium |
| | Proteinate); Magnesium (Magnesium Sulfate); Magnesium Acetate; Magnesium |
| | Alginate; Magnesium Amino Acid Chelate; Magnesium Ascorbate; Magnesium |
| | Aspartate; Magnesium Bicarbonate; Magnesium Borogluconate; Magnesium |
| | Carbonate; Magnesium Carbonate (Dolomite); Magnesium Chloride; |
| | Magnesium Chloride (Magnesium Chloride Hexahydrate); Magnesium Chloride |
| | (Magnesium Chloride); Magnesium Chloride Anhydrous; Magnesium Citrate; |
| | Magnesium Gluconate; Magnesium Glycerophosphate; Magnesium Hvp |
| | Chelate; Magnesium Hvp Chelate (Magnesium Hvp Chelate); Magnesium Hvp |
| | Chelate (Magnesium Oxide); Magnesium Hydroxide; Magnesium Hydroxide |
| | (Dried Aluminum Hydroxide Gel); Magnesium Hypophosphite; Magnesium |
| | Malate; Magnesium Oxide; Magnesium Oxide (Magnesium Citrate); Magnesium |
| | Oxide (Magnesium Oxide); Magnesium Pemoline; Magnesium Phosphate |
| | Dibasic; Magnesium Pidolate; Magnesium Proteinate; Magnesium Salicylate; |
| | Magnesium Silicate; Magnesium Stearate; Magnesium Sulfate; Magnesium |
| | Sulfate: Polyethylene Glycol 3350: Potassium Chloride: Potassium Sulfate: |
| | Sodium Bicarbonate: Sodium Chloride: Sodium Sulfate; Magnesium Sulfate: |
| | Potassium Sulfate: Sodium Sulfate; Magnesium Trisilicate; Magnolia |
| | Grandiflora; Malabar Nut; Malachite Green; Malic Acid; Mallow; Malt; Malt |
| | Extract; Maltase; Malus Pumila; Mamma; Mancinella; Mandragora Officinarum; |
| | Mangafodipir Trisodium; Manganese; Manganese (Manganese Amino Acid |
| | Chelate); Manganese (Manganese Carbonate); Manganese (Manganese Chloride; |
| | Manganese (Manganese Chloride); Manganese (Manganese Citrate; Manganese |
| | (Manganese Citrate); Manganese (Manganese Fumarate; Manganese |
| | (Manganese Fumarate); Manganese (Manganese Gluconate; Manganese |
| | (Manganese Gluconate); Manganese (Manganese Glutarate); Manganese |
| | (Manganese Glycerophosphate); Manganese (Manganese Hvp Amino Acid |
| | Chelate); Manganese (Manganese Hvp Chelate; Manganese (Manganese Hvp |
| | Chelate); Manganese (Manganese Malate; Manganese (Manganese Maleate); |
| | Manganese (Manganese Organic Complex); Manganese (Manganese Oxide); |
| | Manganese (Manganese Proteinate); Manganese (Manganese Succinate); |
| | Manganese (Manganese Sulfate; Manganese (Manganese Sulfate); Manganese |
| | (Sea Proteinate); Manganese Acetate; Manganese Aspartate; Manganese |
| | Carbonate; Manganese Chloride; Manganese Citrate; Manganese Dioxide; |
| | Manganese Gluconate; Manganese Glycerophosphate; Manganese Hvp Chelate; |
| | Manganese Hvp Chelate (Manganese Gluconate); Manganese Hypophosphite; |
| | Manganese Oxide; Manganese Phytate; Manganese Proteinate; Manganese |
| | Sulfate; Manganese Sulfate (Manganese Sulfate); Mango; Manna; Mannitol; |
| | Maple; Maprotiline Hydrochloride; Maraviroc; Marbofloxacin; Maropitant |
| | (Maropitant Citrate); Marsdenia Cundurango; Marsh Elder; Massa Fermentata |
| | Medicinalis; Masterwort; Mate; Mazindol; Measles Virus Vaccine Live |
| | Attenuated; Measles Virus Vaccine Live Attenuated (Edmonston B Strain); |
| | Mebendazole; Mebezonium Iodide; Mebrofenin; Mechlorethamine |
| | Hydrochloride; Meclizine Hydrochloride; Meclofenamic Acid; Medetomidine |
| | Hydrochloride; Medium Chain Triglycerides (Mct); Medorrhinum; |
| | Medrogestone; Medronate Sodium; Medronic Acid; Medroxyprogesterone |
| | Acetate; Medrysone; Medusa; Mefenamic Acid; Mefloquine (Mefloquine |
| | Hydrochloride); Mefloquine Hydrochloride; Megestrol Acetate; Meglumine |
| | Iodipamide; Meglumine Ioxitalamate; Melanoma Lysate; Melarsomine |
| | Dihydrochloride; Melatonin; Melengestrol Acetate; Melilot; Melilotus Alba; |
| | Melissa Officinalis; Meloxicam; Melphalan; Melphalan (Melphalan |
| | Hydrochloride); Memantine Hydrochloride; Menadione; Menadione (Menadione |
| | Sodium Bisulfite); Menadione Sodium Bisulfite; Menadione Sodium Bisulfite |
| | (Vitamin K3); Meningococcal Group A Oligosaccharide; Meningococcal Group |
| | C Oligosaccharide; Meningococcal Group W-135 Oligosaccharide; |
| | Meningococcal Group Y Oligosaccharide; Meningococcal Polysaccharide |
| | Antigen Group A; Meningococcal Polysaccharide Antigen Group C; |
| | Meningococcal Polysaccharide Antigen Group W-135; Meningococcal |
| | Polysaccharide Antigen Group Y; Meningococcal Polysaccharide Vaccine Grp |
| | C; Meningococcal Polysaccharide-Diphtheria Toxoid Conjugate; Menotropins; |
| | Mentha; Mentha Arvensis; Mentha Arvensis Leaf Oil; Mentha Pulegium; |
| | Menthol; Menthol (Peppermint Oil); Menthyl Salicylate; Menyanthes; |
| | Meperidine Hydrochloride; Mephenesin; Mephentermine (Mephentermine |
| | Sulfate); Mephitis Mephitica; Mepivacaine Hydrochloride; Mepolizumab; |
| | Meprobamate; Mequinol; Meradimate; Merbromin; Mercaptobenzothiazole; |
| | Mercaptopurine; Mercuric Chloride; Mercuric Iodide; Mercuric Sulfide Red; |
| | Mercurous Chloride; Mercury; Meropenem; Meropenem (Meropenem |
| | Trihydrate); Mesalazine; Mesna; Mesoridazine (Mesoridazine Besylate); |
| | Mestranol; Metallum Album Homeo Chemical; Metformin Hydrochloride; |
| | Metformin Hydrochloride: Pioglitazone Hydrochloride; Metformin |
| | Hydrochloride: Rosiglitazone Maleate; Metformin Hydrochloride: Saxagliptin |
| | Hydrochloride; Metformin Hydrochloride: Sitagliptin Phosphate; Methacholine |
| | Chloride; Methadone Hydrochloride; Methamphetamine Hydrochloride; |
| | Methandrostenolone; Methanol; Methapyrilene Hydrochloride; Methazolamide; |
| | Methenamine; Methenamine Hippurate; Methenamine Mandelate; Methionine; |
| | Methocarbamol; Methohexital Sodium; Methotrexate; Methotrexate |
| | (Methotrexate Disodium); Methotrexate (Methotrexate Sodium); Methotrexate |
| | Sodium; Methotrimeprazine (Methotrimeprazine Hydrochloride); |
| | Methotrimeprazine (Methotrimeprazine Maleate); Methoxamine Hydrochloride; |
| | Methoxsalen; Methoxy Polyethylene Glycol-Epoetin Beta; Methoxyflurane; |
| | Methscopolamine Bromide; Methsuximide; Methyclothiazide; Methyl |
| | Aminolevulinate (Methyl Aminolevulinate Hydrochloride); Methyl Dodecyl |
| | Benzyl Trimethyl Ammonium Chloride; Methyl Heptyl Colamino Formylmethyl |
| | Pyridinium Chl; Methyl Nicotinate; Methyl Salicylate; Methyl Violet; |
| | Methylbenzethonium Chloride; Methylcellulose; Methylchloroisothiazolinone; |
| | Methyldopa; Methyldopate Hydrochloride; Methylene Blue; Methylnaltrexone |
| | Bromide; Methylparaben; Methylphenidate; Methylphenidate Hydrochloride; |
| | Methylprednisolone; Methylprednisolone (Methylprednisolone Sodium |
| | Succinate); Methylprednisolone Acetate; Methylprednisolone Sodium Succinate; |
| | Methyltestosterone; Methysergide Maleate; Metoclopramide Hydrochloride; |
| | Metocurine Iodide; Metolazone; Metomidate Hydrochloride; Metoprolol |
| | Tartrate; Metreleptin; Metronidazole; Mexiletine Hydrochloride; Mezereum; |
| | Mica; Micafungin Sodium; Miconazole (Miconazole Nitrate); Miconazole |
| | Nitrate; Midazolam; Midazolam (Midazolam Hydrochloride); Midodrine |
| | Hydrochloride; Midostaurin; Mifepristone; Migalastat (Migalastat |
| | Hydrochloride); Miglustat; Milbemycin Oxime; Milk Thistle; Milk Whole; |
| | Milnacipran Hydrochloride; Milrinone (Milrinone Lactate); Mimulus Guttatus; |
| | Mineral Oil; Mineral Oil Light; Minerals Homeopathic; Minocycline |
| | (Minocycline Hydrochloride); Minocycline Hydrochloride; Minoxidil; |
| | Mipomersen Sodium; Mirabegron; Miristalkonium Chloride; Mirtazapine; |
| | Misoprostol; Mistletoe; Mitchella Repens; Mitomycin; Mitotane; Mitoxantrone |
| | (Mitoxantrone Hydrochloride); Mitoxantrone Hydrochloride; Mivacurium |
| | (Mivacurium Chloride); Mixed Salts Amphetamine; Mixed Tocopherols; |
| | Moclobemide; Modafinil; Modified Grass Tyrosine Adsorbate; Modified |
| | Ragweed Tyrosine Adsorbate; Modified Tree Tyrosine Adsorbate; Modified |
| | Vaccinia Virus (Ankara-Bavarian Nordic); Molybdenum; Molybdenum (Kelp; |
| | Molybdenum (Molybdenum Aspartate); Molybdenum (Molybdenum Citrate; |
| | Molybdenum (Molybdenum Citrate); Molybdenum (Molybdenum Fumarate); |
| | Molybdenum (Molybdenum Hvp Chelate); Molybdenum (Molybdenum Organic |
| | Complex); Molybdenum (Molybdenum Proteinate); Molybdenum (Molybdenum |
| | Succinate); Molybdenum (Molybdenum Trioxide); Molybdenum (Molybdenum |
| | Yeast); Molybdenum (Sodium Molybdate); Molybdenum (Yeast); Molybdenum |
| | Hvp Chelate; Molybdenum Proteinate; Molybdenum Trioxide; Mometasone |
| | Furoate; Mometasone Furoate (Mometasone Furoate Monohydrate); Momordica |
| | Balsamina; Monensin; Monensin (Monensin Sodium); Monensin Sodium; |
| | Monobenzone; Monophosphoryl Lipid A; Monosodium Edetate; Montelukast |
| | (Montelukast Sodium); Morantel Tartrate; Moroctocog Alfa; Morphine (Opium); |
| | Morphine Hydrochloride; Morphine Sulfate; Morphine Sulfate: Naltrexone |
| | Hydrochloride; Mosquito; Motherwort Common; Mountain Avens; |
| | Moxetumomab Pasudotox-Tdfk; Moxidectin; Moxifloxacin (Moxifloxacin |
| | Hydrochloride); Moxifloxacin Hydrochloride; Mucilage; Mugwort; Mulberry; |
| | Mumps Vaccine Inactivated; Mumps Virus Vaccine Live Attenuated (Jeryl Lynn |
| | Strain); Mupirocin; Mupirocin (Mupirocin Calcium); Murex Purpurea; |
| | Muromonab-Cd3; Muscle (Extract); Mushroom; Musk; Mustard; Mustard Black; |
| | Mycophenolate Mofetil; Mycophenolate Mofetil (Mycophenolate Mofetil |
| | Hydrochloride); Mycophenolate Mofetil Hydrochloride; Mycophenolic Acid; |
| | Mycophenolic Acid (Mycophenolate Sodium); Mygale; Myocarde; Myosotis |
| | Arvensis; Myristamidopropyl Dimethylamine; Myristica (Nutmeg); Myristica |
| | Oil; Myroxylon Balsamum; Myrrh; Myrtus Communis |
| N | N; N-Acetyl-L-Tyrosine; N-Cyclohexyl-2-Benzothiazolesulfenamide; N- |
| | Cyclohexyl-N′-Phenyl-Para-Phenylenediamine; N-Dodecyl-N; N- |
| | Hydroxymethylsuccinimide; N-Isopropyl-N′-Phenyl-Para-Phenylenediamine; |
| | N-Tetradecyl-N; Nabilone; Nabumetone; Nadidum; Nadolol; Nadroparin |
| | Calcium; Nafarelin (Nafarelin Acetate); Nafarelin Acetate; Nafcillin (Nafcillin |
| | Sodium); Naftifine Hydrochloride; Naja; Nalbuphine Hydrochloride; |
| | Naldemedine Tosylate; Nalidixic Acid; Naloxegol (Naloxegol Oxalate); |
| | Naloxegol Oxalate; Naloxone (Naloxone Hydrochloride Dihydrate); Naloxone |
| | Hydrochloride; Naloxone Hydrochloride (Naloxone Hydrochloride Dihydrate); |
| | Naloxone Hydrochloride: Oxycodone Hydrochloride; Naltrexone; Naltrexone |
| | Hydrochloride; Naltrexone Hydrochloride: Oxycodone Hydrochloride; |
| | Nandrolone Decanoate; Naphazoline Hydrochloride; Naphazoline Nitrate; |
| | Naphthalene; Naproxen; Naproxen (Naproxen; Naproxen (Naproxen Sodium; |
| | Naproxen (Naproxen Sodium); Naproxen Sodium; Naproxen Sodium: |
| | Sumatriptan Succinate; Narasin; Naratriptan (Naratriptan Hydrochloride); |
| | Natalizumab; Nateglinide; Neatsfoot Oil; Nebivolol (Nebivolol Hydrochloride); |
| | Necitumumab; Nedocromil Sodium; Nefazodone Hydrochloride; Neisseria |
| | Catarrhalis; Neisseria Meningitidis Grp B Recombinant Lipoprotein 2086 |
| | Subfamily A; Neisseria Meningitidis Grp B Recombinant Lipoprotein 2086 |
| | Subfamily B; Neisseria Meningitidis Outer Membrane Protein Complex; |
| | Nelarabine; Nelfinavir (Nelfinavir Mesylate); Neomycin; Neomycin (Neomycin |
| | Sulfate); Neomycin Sulfate; Neon; Neostigmine Bromide; Neostigmine |
| | Methylsulfate; Nepafenac; Neratinib Maleate; Nesiritide; Netilmicin Sulfate; |
| | Netupitant; Nevirapine; New Jersey Tea; Niacinamide Ascorbate; Niaouli Oil; |
| | Nicarbazin; Nicardipine Hydrochloride; Nickel; Nickel (Nickel Sulfate); Nickel |
| | Carbonate Hydroxide; Nickel Sulfate; Nicoboxil; Nicotinamide; Nicotinamide |
| | (Niacin Ascorbate); Nicotinamide (Niacinamide Ascorbate); Nicotinamide |
| | (Nicotinamide; Nicotinamide (Nicotinamide Ascorbate); Nicotinamide |
| | (Riboflavin 5′-Phosphate Sodium); Nicotinamide Ascorbate; Nicotine; Nicotinic |
| | Acid; Nicotinic Acid (Inositol Niacinate); Nicotinic Acid (Niacinamide |
| | Ascorbate); Nicotinic Acid (Nicotinamide); Nicotinic Acid (Yeast); Nifedipine; |
| | Nilotinib (Nilotinib Hydrochloride Monohydrate); Nilotinib Hydrochloride; |
| | Nilutamide; Nimodipine; Ningo Figwort Root; Nintedanib (Nintedanib Esilate); |
| | Nitarsone; Nitenpyram; Nitisinone; Nitrazepam; Nitric Acid; Nitric Oxide; |
| | Nitrobenzene; Nitrofurantoin; Nitrofurazone; Nitrogen; Nitroglycerin; |
| | Nitroscanate; Nitrous Oxide; Nivolumab; Nizatidine; Non Pollen; Nonacog |
| | Gamma; Nonoxinol; Nonyl Phenoxy Polyoxyethylene Ethanol-Iodine Complex; |
| | Nonyl Phenoxypoly Ethoxy Ethanol-Iodine; Nonyl Phenoxypolyethoxyethanol- |
| | Iodine Complex; Norelgestromin; Norepinephrine (Norepinephrine Bitartrate); |
| | Norethindrone; Norethindrone Acetate; Norfloxacin; Norgestimate; Norgestrel; |
| | Norgestrel (Norgestrel); Normethadone Hydrochloride; Nortriptyline |
| | (Nortriptyline Hydrochloride); Nortriptyline Hydrochloride; Novobiocin; |
| | Novobiocin (Novobiocin Sodium); Nuphar Luteum; Nusinersen (Nusinersen |
| | Sodium); Nux Moschata; Nux Vomica; Nylidrin Hydrochloride; Nystatin |
| O | O-Benzyl-P-Chlorophenol Potassium Salt; O-Phenylphenol; O-Phenylphenol |
| | Potassium Salt; Oak Moss; Oats; Obeticholic Acid; Obinutuzumab; Ocimum |
| | Sanctum; Oclacitinib (Oclacitinib Maleate); Ocrelizumab; Ocriplasmin; |
| | Octadecyl Dimethyl Benzyl Ammonium Chloride; Octanoic Acid; Octinoxate; |
| | Octisalate; Octocrylene; Octreotide (Octreotide Acetate); Octyl Decyl Dimethyl |
| | Ammonium Chloride; Octyl Dimethyl Ammonium Chloride; Oenothera Biennis; |
| | Ofatumumab; Ofloxacin; Oil Of Anise; Oil Of Cajeput; Oil Of Camphor; Oil Of |
| | Camphor Rectified; Oil Of Cedar Leaf; Oil Of Cedar Wood; Oil Of Chamomile |
| | Roman; Oil Of Citronella; Oil Of Dill; Oil Of Fir; Oil Of Fleabane; Oil Of |
| | Hemlock Canadian; Oil Of Honeysuckle; Oil Of Lemon; Oil Of Mustard |
| | Expressed; Oil Of Orange; Oil Of Pine Needles; Oil Of Santal; Oil Of Sassafras; |
| | Oil Of Sweet Almond; Oil Of Thyme; Oil Of Turpentine; Oil Of Wormwood; |
| | Okra; Olanzapine; Olanzapine (Olanzapine Tartrate); Olanzapine Pamoate; |
| | Olaparib; Olaratumab; Old Tuberculin (Tuberculin Tine Test); Olea Europaea; |
| | Olea Europea Homeo Chemical; Oleander; Oleic Acid; Oleic Acid (Borage Oil); |
| | Oleic Acid (Cod Liver Oil); Oleic Acid (Evening Primrose Oil); Oleic Acid (Fish |
| | Oil); Oleic Acid (Soybean Oil); Oleum Animale; Olibanum; Olive Oil; |
| | Olmesartan Medoxomil; Olodaterol (Olodaterol Hydrochloride); Olodaterol |
| | Hydrochloride; Olodaterol Hydrochloride: Tiotropium Bromide; Olopatadine |
| | (Olopatadine Hydrochloride); Olsalazine Sodium; Omacetaxine Mepesuccinate; |
| | Omalizumab; Ombitasvir; Ombitasvir: Paritaprevir: Ritonavir; Omeprazole; |
| | Omeprazole (Omeprazole Magnesium); Omeprazole Magnesium; Omeprazole: |
| | Sodium Bicarbonate; Onabotulinumtoxina; Ondansetron; Ondansetron |
| | (Ondansetron Hydrochloride Dihydrate); Ondansetron (Ondansetron |
| | Hydrochloride); Ondansetron Hydrochloride Dihydrate; Onion; Ononis Spinosa; |
| | Onopordum; Onosmodium Virginianum; Oophorinum; Ophiopogon Japonicus; |
| | Opium; Orange; Orange Peel Bitter; Orange Peel Sweet; Orbifloxacin; Orchitic |
| | Extract; Orchitinum; Orciprenaline Sulfate; Oreodaphne Californica; Orgotein; |
| | Origanum Majorana; Orlistat; Ormetoprim; Ornithogalum Umbellatum; |
| | Orphenadrine Citrate; Orphenadrine Hydrochloride; Orris; Ortho- |
| | Phthalaldehyde; Orthosiphon Leaf; Oseltamivir (Oseltamivir Phosphate); |
| | Osimertinib (Osimertinib Mesylate); Osmium Metallicum; Outer Membrane |
| | Vesicles (Neisseria Meningitidis Group B Nz98/254 Strain); Ova Tosta; Ovary |
| | Tissue; Ovi Gallinae Pellicula; Ovine Follicle Stimulating Hormone (Ofsh); Ox |
| | Bile Extract; Ox Lip Flower; Oxalic Acid; Oxaliplatin; Oxalis Acetosella; |
| | Oxantel (Oxantel Pamoate); Oxantel Pamoate; Oxaprozin; Oxaprozin Potassium; |
| | Oxazepam; Oxcarbazepine; Oxetacaine; Oxfendazole; Oxibendazole; |
| | Oxiconazole (Oxiconazole Nitrate); Oxprenolol Hydrochloride; Oxtriphylline; |
| | Oxybenzone; Oxybutynin; Oxybutynin Chloride; Oxycodone; Oxycodone |
| | Hydrochloride; Oxydendrum Arboreum; Oxygen; Oxymetazoline |
| | Hydrochloride; Oxymetholone; Oxymorphone Hydrochloride; |
| | Oxyphenbutazone; Oxyquinoline; Oxyquinoline Benzoate; Oxyquinoline |
| | Sulfate; Oxytetracycline; Oxytetracycline (Oxytetracycline Dihydrate); |
| | Oxytetracycline (Oxytetracycline Hydrochloride); Oxytetracycline |
| | Hydrochloride; Oxytetracycline Hydrochloride (Oxytetracycline Dihydrate); |
| | Oxytocin; Oxyurus Vermicularis Homeopathic Animal; Oyster Shells; |
| | Ozenoxacin |
| P | P-Hydroxyephedrine Hydrochloride; P-Phenylenediamine; P-Tert-Amylphenol |
| | Potassium Salt; P-Tert-Butylphenol Formaldehyde Resin; P-Tert-Pentylphenol; |
| | Pacific Weeds; Paclitaxel; Padimate O; Paeonia Officinalis; Palbociclib; |
| | Palifermin; Paliperidone; Paliperidone (Paliperidone Palmitate); Palivizumab; |
| | Palladium; Palmitic Acid; Palmitic Acid (Borage Oil); Palmitic Acid (Evening |
| | Primrose Oil); Palmitic Acid (Fish Oil); Palmitic Acid (Soybean Oil); Palmitoleic |
| | Acid (Fish Oil); Palonosetron (Palonosetron Hydrochloride); Pamabrom; |
| | Pamidronate Disodium; Pancreas; Pancreatic (Ext.) Etc.; Pancreatin; Pancreatin |
| | (Protease; Pancrelipase; Pancrelipase (Amylase:Lipase:Protease); Pancuronium |
| | Bromide; Panitumumab; Panobinostat Lactate; Pantoprazole (Pantoprazole |
| | Magnesium Hemipentahydrate); Pantoprazole (Pantoprazole Magnesium); |
| | Pantoprazole (Pantoprazole Sodium Sesquihydrate); Pantoprazole (Pantoprazole |
| | Sodium); Pantoprazole Sodium; Papain; Papain (Papaya); Papain (Papayotin); |
| | Papaverine; Papaverine Hydrochloride; Papaya; Parachlorophenol; Paraffin; |
| | Paraformaldehyde; Paraldehyde; Parathyroid Hormone; Pareira Brava; |
| | Paricalcitol; Paris Quadrifolia; Paritaprevir; Paromomycin (Paromomycin |
| | Sulfate); Parotid Gland Tissue; Paroxetine (Paroxetine Hydrochloride; |
| | Paroxetine (Paroxetine Hydrochloride Acetone Solvate); Paroxetine (Paroxetine |
| | Hydrochloride Hemihydrate); Paroxetine (Paroxetine Hydrochloride); |
| | Paroxetine Hydrochloride; Paroxetine Mesylate; Parsley; Parsley Piert; |
| | Parthenolide (Feverfew); Pasireotide (Pasireotide Diaspartate); Pasireotide |
| | (Pasireotide Pamoate); Pasireotide Diaspartate; Pasireotide Pamoate; Passion |
| | Flower; Passion Flower (Passiflora Incarnata); Passion Flower (Passion Flower); |
| | Patent Blue Sodium; Patiromer (Patiromer Sorbitex Calcium); Pazopanib |
| | (Pazopanib Hydrochloride); Pazopanib Hydrochloride; Peanut Oil; Pectin; |
| | Pectinase; Pediculus Capitis; Peforelin (Peforelin Acetate); Pegaptanib; |
| | Pegaspargase; Pegbovigrastim; Pegfilgrastim; Peginesatide Acetate; |
| | Peginterferon Alfa-2a; Peginterferon Alfa-2b; Peginterferon Beta-1a; |
| | Pegloticase; Pegvaliase-Pqpz; Pegvisomant; Pembrolizumab; Pemetrexed |
| | (Pemetrexed Disodium Hemipentahydrate); Pemetrexed (Pemetrexed |
| | Disodium); Penciclovir; Penghawar; Penicillin; Penicillin (Penicillin G |
| | Potassium); Penicillin (Penicillin G Procaine); Penicillin G; Penicillin G |
| | (Penicillin G Potassium); Penicillin G (Penicillin G Procaine); Penicillin G |
| | (Penicillin G Sodium); Penicillin G Benzathine; Penicillin G Potassium; |
| | Penicillin G Procaine; Penicillin G Sodium; Penicillin V; Penicillin V (Penicillin |
| | V Benzathine); Penicillin V (Penicillin V Potassium); Penicillin V Benzathine; |
| | Penicillin V Potassium; Pentaerythritol Tetranitrate; Pentagastrin; Pentamidine |
| | Isetionate; Pentastarch; Pentazocine (Pentazocine Hydrochloride); Pentazocine |
| | (Pentazocine Lactate); Pentetate Calcium Trisodium; Pentetate Indium Disodium |
| | In 111; Pentetic Acid; Pentetreotide; Pentobarbital; Pentobarbital Sodium; |
| | Pentosan Polysulfate Sodium; Pentostatin; Pentoxifylline; Peppermint; |
| | Peppermint Oil; Pepsin; Peptone; Peracetic Acid; Peramivir; Perampanel; |
| | Perflutren; Pergolide (Pergolide Mesylate); Periciazine; Perindopril Arginine; |
| | Perindopril Erbumine; Periwinkle; Permethrin; Perphenazine; Pertactin; |
| | Pertactin Adsorbed; Pertussis Toxoid; Pertussis Toxoid Adsorbed; Pertussis |
| | Vaccine; Pertuzumab; Peruvian Balsam; Petrolatum; Petroleum; Phellandrium |
| | Aquaticum; Phenazopyridine Hydrochloride; Phenelzine (Phenelzine Sulfate); |
| | Phenelzine Sulfate; Pheniramine Maleate; Phenobarbital; Phenobarbital Sodium; |
| | Phenol; Phenol (Phenolate Sodium); Phenolate Sodium; Phenolphthalein; |
| | Phenothiazine; Phenoxyethanol; Phenoxyisopropanol; Phentermine |
| | Hydrochloride; Phentermine Hydrochloride: Topiramate; Phentermine Resin; |
| | Phentolamine Mesylate; Phenyl Salicylate; Phenylalanine; Phenylbutazone; |
| | Phenylbutazone (Phenylbutazone Sodium); Phenylbutazone Sodium; |
| | Phenylephrine (Phenylephrine Hydrochloride); Phenylephrine Hydrochloride; |
| | Phenylmercuric Nitrate (Basic); Phenylpropanolamine Bitartrate; |
| | Phenylpropanolamine Hydrochloride; Phenyltoloxamine; Phenyltoloxamine |
| | Citrate; Phenytoin; Phenytoin Sodium; Phleum Pratence; Phloridzinum; |
| | Phosphatidyl Choline; Phosphatidyl Choline (Soybean); Phospholipid; |
| | Phosphoric Acid; Phosphorus; Phosphorus (Bone Ash); Phosphorus (Bone |
| | Meal); Phosphorus (Calcium Glycerophosphate); Phosphorus (Calcium |
| | Hypophosphite); Phosphorus (Calcium Phosphate; Phosphorus (Calcium |
| | Phosphate (Dibasic); Phosphorus (Calcium Phosphate (Dibasic)); Phosphorus |
| | (Calcium Phosphate (Tribasic)); Phosphorus (Calcium Phosphate Monobasic); |
| | Phosphorus (Calcium Phosphate); Phosphorus (Dicalcium Orthophosphate); |
| | Phosphorus (Durapatite); Phosphorus (Hydroxyapatite); Phosphorus |
| | (Magnesium Hypophosphite); Phosphorus (Phosphorus Amino Acid Chelate); |
| | Phosphorus (Phosphorus Hvp Chelate; Phosphorus (Phosphorus Hvp Chelate); |
| | Phosphorus (Phosphorus Hydroxyapatite); Phosphorus (Phosphorus Proteinate); |
| | Phosphorus (Potassium Hypophosphite; Phosphorus (Potassium Phosphate |
| | Dibasic); Phosphorus (Potassium Phosphate Monobasic; Phosphorus (Potassium |
| | Phosphate); Phosphorus (Sodium Hypophosphite); Phosphorus (Sodium |
| | Phosphate Dibasic); Phosphorus (Sodium Phosphate); Phosphorus Amino Acid |
| | Chelate; Phosphorus Hvp Chelate; Phosphorus Proteinate; Phosphorus |
| | Triiodatus; Physostigmine Salicylate; Physostigminum; Phytase; Phytolacca |
| | Decandra; Pibrentasvir; Pichi; Picric Acid; Pilewort Herb; Pilocarpine; |
| | Pilocarpine Hydrochloride; Pilocarpine Nitrate; Pilocarpus; Pimecrolimus; |
| | Pimobendan; Pimozide; Pimpinella Saxifraga; Pinaverium Bromide; Pindolol; |
| | Pine Oil; Pine Tar; Pine Tar Oil; Pineapple; Pinene; Pinus Sylvestris; |
| | Pioglitazone (Pioglitazone Hydrochloride); Pioglitazone Hydrochloride; |
| | Piperacillin (Piperacillin Sodium); Piperazine; Piperazine (Piperazine Adipate); |
| | Piperazine (Piperazine Citrate); Piperazine (Piperazine Hydrobromide); |
| | Piperazine (Piperazine Hydrochloride); Piperazine (Piperazine Sulfate); |
| | Piperazine Adipate; Piperazine Citrate; Piperazine Dihydrochloride; Piperazine |
| | Hydrochloride; Piperazine Phosphate; Piperonyl Butoxide; Pipotiazine |
| | Palmitate; Pipradrol Hydrochloride; Pirbuterol (Pirbuterol Acetate); Pirenzepine |
| | Hydrochloride; Pirfenidone; Pirlimycin (Pirlimycin Hydrochloride); Piroxicam; |
| | Piroxicam B-Cyclodextrin; Pitcher Plant; Pituitary Posterior; Pituitary Whole; |
| | Pivampicillin; Pivmecillinam (Pivmecillinam Hydrochloride); Pivmecillinam |
| | Hydrochloride; Pizotifen (Pizotifen Malate); Placenta; Plantago Cordata; |
| | Plantago Major; Plantago Seed; Plasma Protein Fraction (Human); |
| | Plasmafibronectin (Cig); Plasminogen; Platanus Occidentalis; Platanus |
| | Orientalis; Platina Homeo Chemical; Platinum; Platinum Chloride; Plecanatide; |
| | Plectranthus Fruticosus; Plerixafor; Plumbago Littoralis; Plumbum Mellitum; |
| | Plumbum Metallicum; Pneumococcal Polysacc. Serot. 18c Conjug. To Tetanus |
| | Toxoid Carrier Prot.; Pneumococcal Polysaccharid Serot. 19f Conjug. To |
| | Diphtheria Toxoid Carrier Prot.; Pneumococcal Polysaccharide Serotype 1; |
| | Pneumococcal Polysaccharide Serotype 1 Conjugated To Protein D Carrier |
| | Protein; Pneumococcal Polysaccharide Serotype 10a; Pneumococcal |
| | Polysaccharide Serotype 11a; Pneumococcal Polysaccharide Serotype 12f; |
| | Pneumococcal Polysaccharide Serotype 14; Pneumococcal Polysaccharide |
| | Serotype 14 Conjugated To Protein D Carrier Protein; Pneumococcal |
| | Polysaccharide Serotype 15b; Pneumococcal Polysaccharide Serotype 17f; |
| | Pneumococcal Polysaccharide Serotype 18c; Pneumococcal Polysaccharide |
| | Serotype 19a; Pneumococcal Polysaccharide Serotype 19f; Pneumococcal |
| | Polysaccharide Serotype 2; Pneumococcal Polysaccharide Serotype 20; |
| | Pneumococcal Polysaccharide Serotype 22f; Pneumococcal Polysaccharide |
| | Serotype 23f; Pneumococcal Polysaccharide Serotype 23f Conjugated To Protein |
| | D Carrier Protein; Pneumococcal Polysaccharide Serotype 3; Pneumococcal |
| | Polysaccharide Serotype 33f; Pneumococcal Polysaccharide Serotype 4; |
| | Pneumococcal Polysaccharide Serotype 4 Conjugated To Protein D Carrier |
| | Protein; Pneumococcal Polysaccharide Serotype 5; Pneumococcal |
| | Polysaccharide Serotype 5 Conjugated To Protein D Carrier Protein; |
| | Pneumococcal Polysaccharide Serotype 6a; Pneumococcal Polysaccharide |
| | Serotype 6b; Pneumococcal Polysaccharide Serotype 6b Conjugated To Protein |
| | D Carrier Protein; Pneumococcal Polysaccharide Serotype 7f; Pneumococcal |
| | Polysaccharide Serotype 7f Conjugated To Protein D Carrier Protein; |
| | Pneumococcal Polysaccharide Serotype 8; Pneumococcal Polysaccharide |
| | Serotype 9n; Pneumococcal Polysaccharide Serotype 9v; Pneumococcal |
| | Polysaccharide Serotype 9v Conjugated To Protein D Carrier Protein; |
| | Pneumococcal Vaccine Polyvalent; Pneumococcus (Various Types Mixture); |
| | Podophyllin; Podophyllotoxin; Podophyllum; Poison Ivy; Poison Oak; Poison |
| | Sumac; Polidocanol; Poliomyelitis Vaccine (Inactivated); Poliovirus Vaccine |
| | Live Orl-Trival Human Type-1; Poliovirus Vaccine Live Orl-Trival Human |
| | Type-2; Poliovirus Vaccine Live Orl-Trival Human Type-3; Polistes Spp Venom |
| | Protein Extract; Pollen; Pollen And Non Pollen; Pollen-English Plantain; |
| | Poloxalene; Poloxamer (Poloxamer 188); Poloxamer Iodine Complex; |
| | Polyalkylene Glycol Ether Iodophor; Polyalkylene Glycol Iodine Complex; |
| | Polyaminopropyl Biguanide; Polycarbophil (Calcium Polycarbophil); |
| | Polyethoxy Polypropoxy Polyethoxy Ethanol - Iodine Complex; Polyethylene |
| | Glycol 3350; Polyethylene Glycol 3350: Potassium Chloride: Sodium |
| | Bicarbonate: Sodium Chloride; Polyethylene Glycol 3350: Potassium Chloride: |
| | Sodium Bicarbonate: Sodium Chloride: Sodium Sulfate Anhydrous; |
| | Polyethylene Glycol 400; Polyethylene Glycol P-Isooctylphenyl Ether; Polygala; |
| | Polygonum Punctatum; Polyhexanide; Polymyxin B; Polymyxin B (Polymyxin |
| | B Sulfate); Polymyxin B (Polymyxin B); Polymyxin B Sulfate; Polyoxyethylene |
| | Glycol Iodine Complex; Polyoxyethylene Lauryl Ether; Polyoxyethylene |
| | Nonylphenol Glycol Iodine Complex; Polyoxypropylene-Polyoxyethylene Block |
| | Copolymer; Polyoxypropylene-Polyoxyethylene Block Copolymer-Iodine |
| | Complex; Polypropoxy Polyethoxyn Nony Phenol Iodine; Polyquaternium 1; |
| | Polyribose Ribitol Phosphate Polysaccharide (Prp); Polysorbate 80; Polystyrene; |
| | Polyvinyl Alcohol; Pomalidomide; Pomegranate Bark; Ponatinib (Ponatinib |
| | Hydrochloride); Ponatinib Hydrochloride; Ponazuril; Poplar; Poplar Bud; Poppy; |
| | Populus Candicans; Populus Tremula; Populus Tremuloides; Poractant Alfa; |
| | Porcine Cartilage; Porcine Factor Viii-C; Porcine Pituitary Luteinizing Hormone; |
| | Porfimer Sodium; Poria Cocos; Posaconazole; Potassium; Potassium |
| | (Glycerophosphate Potassium; Potassium (Glycerophosphate Potassium); |
| | Potassium (Kelp; Potassium (Kelp); Potassium (Potassium Aminoacid Chelate); |
| | Potassium (Potassium Ascorbate); Potassium (Potassium Aspartate); Potassium |
| | (Potassium Bicarbonate); Potassium (Potassium Carbonate; Potassium |
| | (Potassium Carbonate); Potassium (Potassium Chloride; Potassium (Potassium |
| | Chloride); Potassium (Potassium Citrate; Potassium (Potassium Citrate); |
| | Potassium (Potassium Gluconate; Potassium (Potassium Gluconate); Potassium |
| | (Potassium Glycerophosphate); Potassium (Potassium Hvp Chelate; Potassium |
| | (Potassium Hvp Chelate); Potassium (Potassium Iodide); Potassium (Potassium |
| | Nitrate); Potassium (Potassium Organic Complex); Potassium (Potassium |
| | Phosphate Dibasic); Potassium (Potassium Proteinate; Potassium (Potassium |
| | Proteinate); Potassium (Potassium Sulfate); Potassium Acetate; Potassium |
| | Aspartate; Potassium Bicarbonate; Potassium Bitartrate; Potassium Bromide; |
| | Potassium Carbonate; Potassium Chlorate; Potassium Chloride; Potassium |
| | Chromate; Potassium Citrate; Potassium Dichromate; Potassium Gluconate; |
| | Potassium Guaiacol Sulphonate; Potassium Hvp Chelate; Potassium Hvp Chelate |
| | (Potassium Gluconate); Potassium Hydroxide; Potassium Hydroxyquinoline |
| | Sulfate; Potassium Iodate; Potassium Iodide; Potassium Nitrate; Potassium |
| | Ortho-Benzyl Parachlorophenate; Potassium Ortho-Phenylphenate; Potassium |
| | Oxalate; Potassium P-Aminobenzoate; Potassium Perchlorate; Potassium |
| | Permanganate; Potassium Peroxymonosulfate; Potassium Perrhenate; Potassium |
| | Phosphate Dibasic; Potassium Phosphate Monobasic; Potassium Proteinate; |
| | Potassium Silicate; Potassium Sodium Tartrate; Potassium Sulfate; Potassium |
| | Tartrate; Potassium Tertiary Amylphenate; Potentilla Anserina; Povidone; |
| | Povidone-Iodine; Povidone-Iodine (Povidone-Iodine); Ppg-12-Peg-50 Lanolin; |
| | Prabotulinumtoxina; Prabotulinumtoxina-Xvfs; Pradofloxacin; Pralatrexate; |
| | Pralidoxime Chloride; Pramipexole Dihydrochloride Monohydrate; Pramlintide |
| | Acetate; Pramoxine Hydrochloride; Prasugrel (Prasugrel Hydrochloride); |
| | Prasugrel Hydrochloride; Pravastatin Sodium; Praziquantel; Prazosin (Prazosin |
| | Hydrochloride); Prazosin Hydrochloride; Prednicarbate; Prednisolone; |
| | Prednisolone (Prednisolone 21-Succinate Sodium); Prednisolone (Prednisolone |
| | Acetate); Prednisolone (Prednisolone Sodium Phosphate); Prednisolone |
| | (Prednisolone Sodium Succinate); Prednisolone Acetate; Prednisolone Sodium |
| | Phosphate; Prednisone; Pregabalin; Prilocaine; Prilocaine Hydrochloride; |
| | Primaquine Phosphate; Primidone; Primrose; Primula Obconica; Primula |
| | Vulgaris; Privet (Leaves); Probenecid; Probucol; Procainamide Hydrochloride; |
| | Procaine Hydrochloride; Procarbazine (Procarbazine Hydrochloride); Procaterol |
| | Hydrochloride Hemihydrate; Prochlorperazine; Prochlorperazine |
| | (Prochlorperazine Maleate); Prochlorperazine (Prochlorperazine Mesylate); |
| | Prochlorperazine Maleate; Procinonide; Procyclidine Hydrochloride; Proflavine |
| | Hemisulfate; Progesterone; Proguanil (Proguanil Hydrochloride); Proguanil |
| | Hydrochloride; Proline; Promazine Hydrochloride; Promethazine; Promethazine |
| | (Promethazine Hydrochloride); Promethazine Hydrochloride; Propafenone |
| | Hydrochloride; Propantheline Bromide; Propiverine Hydrochloride; Propofol; |
| | Propolis; Propoxyphene Hydrochloride; Propoxyphene Napsylate; Propranolol |
| | (Propranolol Hydrochloride); Propranolol Hydrochloride; Propyl Alcohol; |
| | Propylene Glycol; Propylene Glycol Diacetate; Propylparaben; Propylthiouracil; |
| | Prostate Tissue; Protamine Sulfate; Protease; Protease (Aspergillus Niger); |
| | Protease (Aspergillus Oryzae); Protein; Protein C; Protein Hydrolysates; Protein |
| | Hydrolysates (Casein); Protein Hydrolysates (Yeast); Protein S; Proteolytic |
| | Enzyme From Pancreas; Proteolytic Enzymes From Carica Papaya; Proteus |
| | Mirabilis Antigen; Protirelin; Protriptyline Hydrochloride; Provitamin A; |
| | Proxymetacaine Hydrochloride; Prucalopride (Prucalopride Succinate); Prune; |
| | Prunus Cerasifera; Prunus Laurocerasus L.; Prunus Spinosa; Pseudoephedrine |
| | Hydrochloride; Pseudoephedrine Sulfate; Psorinum; Ptelea Trifoliata; Pueraria |
| | Lobata; Puerparia L.O.; Pulegium Oil; Pulex Irritans; Pulmo Vulpis; Pulsatilla; |
| | Purified Protein Derivative Battey Bacillus; Purified Protein Derivative Of |
| | Tuberculin; Purple Willow; Pyrantel (Pyrantel Pamoate); Pyrantel Pamoate; |
| | Pyrantel Tartrate; Pyrazinamide; Pyrethrins; Pyrethrins (Pyrethrum); Pyrethrum; |
| | Pyridostigmine Bromide; Pyridoxal-5-Phosphate; Pyridoxine Hydrochloride; |
| | Pyrilamine Maleate; Pyrimethamine; Pyrithione Disulfide; Pyrithione Zinc; |
| | Pyrvinium (Pyrvinium Pamoate); |
| Q | Quassia; Quaternium-15; Quazepam; Quercus Alba; Quercus Glandium Spiritus; |
| | Quercus Robur; Quetiapine (Quetiapine Fumarate); Quetiapine Fumarate; |
| | Quinagolide (Quinagolide Hydrochloride); Quinapril (Quinapril Hydrochloride); |
| | Quince; Quinidine Acid Sulfate; Quinidine Gluconate; Quinidine |
| | Phenylethylbarbiturate; Quinidine Polygalacturonate; Quinidine Sulfate; |
| | Quinine; Quinine Dihydrochloride; Quinine Hydrochloride; Quinine Sulfate; |
| | Quinupristin |
| R | Rabeprazole Sodium; Rabies Immunoglobulin (Human); Rabies Vaccine |
| | Inactivated (Human Diploid-Cell Culture); Rabies Vaccine Inactivated (Purified |
| | Chick Embryo Cell Culture); Racemic Epinephrine Hydrochloride; Ractopamine |
| | Hydrochloride; Radish; Radium Bromide; Radium Ra-223 Dichloride; Radix |
| | Angelicae Pubescentis; Radix Dispaci; Radix Gentiana Macrophyllae; Radix |
| | Ledebouriella Seseloides; Ragweed Giant; Ragweed Yellow; Raloxifene |
| | Hydrochloride; Raltegravir (Raltegravir Potassium); Raltitrexed (Raltitrexed |
| | Disodium); Ramelteon; Ramipril; Ramucirumab; Ranibizumab; Ranitidine |
| | (Ranitidine Hydrochloride); Ranitidine Bismuth Citrate; Ranitidine |
| | Hydrochloride; Ranunculus Acris; Ranunculus Bulbosus; Ranunculus Scleratus; |
| | Rasagiline (Rasagiline Mesylate); Rasburicase; Raspberry (Rubus Idaeus); |
| | Rattlesnake; Ravulizumab-Cwvz; Recombinant Cholera Toxin B Subunit; |
| | Recombinant Human Coagulation Factor Ix; Recombinant Human |
| | Papillomavirus Type 11 L1 Protein; Recombinant Human Papillomavirus Type |
| | 16 L1 Protein; Recombinant Human Papillomavirus Type 18 L1 Protein; |
| | Recombinant Human Papillomavirus Type 31 L1 Protein; Recombinant Human |
| | Papillomavirus Type 33 L1 Protein; Recombinant Human Papillomavirus Type |
| | 45 L1 Protein; Recombinant Human Papillomavirus Type 52 L1 Protein; |
| | Recombinant Human Papillomavirus Type 58 L1 Protein; Recombinant Human |
| | Papillomavirus Type 6 L1 Protein; Recombinant Neisseria Meningitidis Group |
| | B Fhbp Fusion Protein; Recombinant Neisseria Meningitidis Group B Nada |
| | Protein; Recombinant Neisseria Meningitidis Group B Nhba Fusion Protein; |
| | Regorafenib; Remestemcel-L; Remifentanil (Remifentanil Hydrochloride); |
| | Renal Extract; Rennin; Repaglinide; Repository Corticotropin; Reserpine; Resina |
| | Laricis; Reslizumab; Resorcinol; Resorcinol Monoacetate; Respiratory Syncytial |
| | Virus Immune Globulin Intravenous (Human); Retapamulin; Reteplase; Retinol |
| | Palmitate; Retinol Palmitate (Retinol Palmitate); Revefenacin; Rhamnus |
| | Californica; Rhizoma Alismatis; Rhizoma Atractylodis; Rhodium Metallicum; |
| | Rhododendron; Rhododendron Chrysanthum; Rhubarb; Rhus Aromatica; |
| | Ribavirin; Ribociclib (Ribociclib Succinate); Riboflavin 5′-Phosphate Sodium; |
| | Riboflavin-5-Phosphate; Ribonucleic Acid; Rice Bran; Ricinus Communis; |
| | Rifabutin; Rifampin; Rifapentine; Rifaximin; Rilpivirine (Rilpivirine |
| | Hydrochloride); Riluzole; Rimabotulinumtoxinb; Rimexolone; Riociguat; |
| | Risankizumab; Risankizumab-Rzaa; Risedronate Sodium; Risedronate Sodium |
| | (Risedronate Sodium Hemi-Pentahydrate); Risedronate Sodium (Risedronate |
| | Sodium Monohydrate); Risperidone; Risperidone (Risperidone Tartrate); |
| | Ritodrine Hydrochloride; Ritonavir; Rituximab; Rituximab-Abbs; Rituximab: |
| | Hyaluronidase (Human Recombinant); Rivaroxaban; Rivastigmine; |
| | Rivastigmine (Rivastigmine Hydrogen Tartrate); Rizatriptan (Rizatriptan |
| | Benzoate); Robenacoxib; Robenidine Hydrochloride; Robinia Pseudoacacia; |
| | Rocuronium Bromide; Rofecoxib; Roflumilast; Rolitetracycline; Romidepsin; |
| | Romifidine; Romiplostim; Romosozumab-Aqqg; Ropinirole (Ropinirole |
| | Hydrochloride); Ropivacaine Hydrochloride; Rosa Canina; Rosa Damascena; |
| | Rose Bengal; Rose Hips; Rose Pollen; Rosemary Oil; Rosiglitazone |
| | (Rosiglitazone Maleate); Rosiglitazone Maleate; Rosin; Rosmarinus Officinalis; |
| | Rosuvastatin (Rosuvastatin Calcium); Rotavirus Vaccine (Rix4414 Strain); |
| | Rotenone; Rotigotine; Rough Pigweed; Roxarsone; Rubella Virus Vaccine Live |
| | Attenuated (Wistar Ra27/3 Strain); Rubia Tinctorium; Rubidium Chloride Rb 82; |
| | Rubus; Rue Oil; Rufinamide; Rumex Acetosa; Rumex Obtusifolius; Rupatadine |
| | (Rupatadine Fumarate); Russian Thistle; Russula Foetens; Ruta Graveolens; |
| | Rutoside; Rutoside (Buckwheat); Ruxolitinib (Ruxolitinib Phosphate); Rye |
| S | Sabadilla; Sabina; Sacubitril (Sacubitril Valsartan Sodium Hydrate Complex); |
| | Safflower; Safflower Oil; Saffron; Safinamide (Safinamide Mesylate); Safrole; |
| | Salbutamol; Salbutamol (Salbutamol Sulfate); Salbutamol Sulfate; Salicin; |
| | Salicylamide; Salicylic Acid; Salinomycin Sodium; Salix Alba; Salix Discolor; |
| | Salix Nigra; Salix Vitellina; Salmeterol (Salmeterol Xinafoate); Salmeterol |
| | Xinafoate; Salmonella Typhi Vi Capsular Polysaccharide Vaccine; Salsalate; Salt |
| | Flower; Salvia; Samarium (153sm) Lexidronam; Sambucus Ebulus; Sambucus |
| | Nigra; Sandalwood White; Sanguinaria Canadensis; Sanguinarina Nitrica |
| | (Homeo); Sanguinarine Chloride; Sanicula; Santonin; Saponaria Officinalis |
| | Extract; Saponins; Sapropterin Dihydrochloride; Saquinavir; Saquinavir |
| | (Saquinavir Mesylate); Saquinavir Mesylate; Sarilumab; Sarolaner; Sarsaparilla; |
| | Sassafras; Satumomab Pendetide; Savin Oil; Saw Palmetto; Saxagliptin |
| | (Saxagliptin Hydrochloride); Saxagliptin Hydrochloride; Scarlet Red; Schinus |
| | Molle; Schisandra; Schizonepeta Tenuifolia; Scleranthus Annuus; Scopolamine; |
| | Scopolamine Hydrobromide; Scouring Rush; Scullcap; Scurvy Grass; SeaWater; |
| | Sealer Protein (Human); Sebelipase Alfa; Secobarbital Sodium; Secretin; |
| | Secukinumab; Sedum Acre; Sedum Telephium; Selamectin; Selegiline; |
| | Selegiline (Selegiline Hydrochloride); Selegiline Hydrochloride; Selenium; |
| | Selenium (D-Alpha Tocopheryl Acetate); Selenium (Dried Yeast); Selenium |
| | (Kelp; Selenium (Kelp); Selenium (Selenious Acid); Selenium (Selenium Amino |
| | Acid Chelate); Selenium (Selenium Aspartate); Selenium (Selenium Chelate); |
| | Selenium (Selenium Citrate; Selenium (Selenium Citrate); Selenium (Selenium |
| | Dioxide); Selenium (Selenium Hvp Amino Acid Chelate); Selenium (Selenium |
| | Hvp Chelate; Selenium (Selenium Hvp Chelate); Selenium (Selenium Organic |
| | Complex); Selenium (Selenium Oxide); Selenium (Selenium Proteinate); |
| | Selenium (Selenium Sulfide); Selenium (Selenium Yeast); Selenium (Sodium |
| | Selenate); Selenium (Sodium Selenite); Selenium (Yeast (Living Cells)); |
| | Selenium (Yeast); Selenium Aspartate; Selenium Citrate; Selenium Hvp Chelate; |
| | Selenium Proteinate; Selenium Proteinate (Selenium Proteinate); Selenium |
| | Sulfide; Selenium Yeast; Selexipag; Self Heal; Semaglutide; Semduramicin |
| | (Semduramicin Sodium); Semen Biotae Orientalis; Sempervivum Tectorum; |
| | Senecio Aureus; Senecio Cineraria; Senega; Senna; Sennosides; Sennosides |
| | (Calcium Sennosides A & B); Sennosides (Senna); Sennosides (Sennosides); |
| | Sepia Officinalis L; Serine; Sertraline (Sertraline Hydrochloride); Sertraline |
| | Hydrochloride; Sevelamer Carbonate; Sevelamer Hydrochloride; Sevoflurane; |
| | Shark Liver Oil; Sheep Sorrel; Shepherds Purse; Siberian Ginseng; Sibutramine |
| | Hydrochloride; Sibutramine Hydrochloride Monohydrate; Sildenafil (Sildenafil |
| | Citrate); Siler Divaricatum; Silica Marina; Silicon; Silicon (Calcium Silicate); |
| | Silicon (Equisetum Arvense); Silicon (Horsetail); Silicon (Silicon Dioxide; |
| | Silicon (Silicon Dioxide); Silicon (Silicon Hvp Chelate); Silicon (Silicon |
| | Proteinate); Silicon (Sodium Metasilicate); Silicon Dioxide; Silicon Dioxide |
| | (Equisetum Arvense); Silicon Dioxide (Silicon Hvp Chelate); Silicon Dioxide |
| | (Sodium Metasilicate); Silicone; Silodosin; Silphium Laciniatum; Siltuximab; |
| | Silver; Silver Acetate; Silver Cyanide; Silver Dihydrogen Citrate; Silver Nitrate; |
| | Silver Phosphate; Silver Sulfadiazine; Simeprevir (Simeprevir Sodium); |
| | Simethicone; Simoctocog Alfa; Simvastatin; Simvastatin: Sitagliptin Phosphate; |
| | Sinapis Alba; Sinapis Arvensis; Sincalide; Sinecatechins; Siponimod Fumaric |
| | Acid; Sirolimus; Sitagliptin (Sitagliptin Phosphate Monohydrate); Sitagliptin |
| | (Sitagliptin Phosphate); Sitagliptin Phosphate; Sitaxsentan Sodium; Skatolum; |
| | Skunk Cabbage; Smallpox Vaccine Dried; Smallpox Vaccine Liquid (Calf); |
| | Sodium; Sodium (4-Dimethylamino-O-Tolyl) Phosphonate Trihydrate; Sodium |
| | (Sodium Acetate; Sodium (Sodium Bicarbonate); Sodium (Sodium Chloride); |
| | Sodium (Sodium Glycerophosphate); Sodium (Sodium Sulfate); Sodium |
| | Acetate; Sodium Acetate (Sodium Acetate Trihydrate); Sodium Acetate |
| | Anhydrous; Sodium Acetate Hemodialysis; Sodium Acetate Trihydrate; Sodium |
| | Acid Citrate; Sodium Alginate; Sodium Alkyl Polyether Sulfate; Sodium |
| | Arsanilate; Sodium Ascorbate; Sodium Auro-Chloride; Sodium Bentonite; |
| | Sodium Benzoate; Sodium Bicarbonate; Sodium Bisulfite; Sodium Borate; |
| | Sodium Bromide; Sodium Cacodylate; Sodium Calcium Edetate; Sodium |
| | Camsylate; Sodium Carbonate (Anhydrous); Sodium Chloride; Sodium Chlorite; |
| | Sodium Citrate; Sodium Citrate Dihydrate; Sodium Diacetate; Sodium |
| | Dichloroisocyanurate; Sodium Dodecylbenzenesulfonate; Sodium Fluoride; |
| | Sodium Fluoride F 18; Sodium Gluconate; Sodium Glutamate; Sodium |
| | Glycerophosphate; Sodium Hexafluorosilicate; Sodium Hydroxide; Sodium |
| | Hypochlorite; Sodium Hypophosphite; Sodium Iodide; Sodium Iodide 123 I; |
| | Sodium Iodide 131 I; Sodium Ioxitalamate; Sodium Lactate; Sodium Lauryl |
| | Ether Sulfate; Sodium Lauryl Sulfate; Sodium Lauryl Sulfoacetate; Sodium |
| | Linear Alkylate Sulfonate; Sodium Metasilicate; Sodium Metavanadate; Sodium |
| | Molybdate; Sodium Monofluorophosphate; Sodium Nitrate; Sodium Nitrite; |
| | Sodium Nitroprusside; Sodium Ortho-Phenylphenate; Sodium Oxybate; Sodium |
| | Oxychlorosene; Sodium P-Dimethyl Amino Orthololuene Phosph Trihyd; |
| | Sodium P-Tert-Amylphenolate; Sodium Para-Aminosalicylate; Sodium |
| | Perborate; Sodium Perborate Monohydrate; Sodium Pertechnetate Tc-99m; |
| | Sodium Phenylbutyrate; Sodium Phosphate (Tribasic); Sodium Phosphate |
| | Dibasic; Sodium Phosphate Monobasic; Sodium Phosphate Monobasic (Sodium |
| | Phosphate Monobasic; Sodium Phosphate, Dibasic Anhydrous: Sodium |
| | Phosphate, Monobasic, Monohydrate; Sodium Phosphate, Dibasic, Anhydrous: |
| | Sodium Phosphate, Monobasic, Monohydrate; Sodium Picosulfate; Sodium |
| | Pidolate; Sodium Polystyrene Sulfonate; Sodium Propionate; Sodium Salicylate; |
| | Sodium Selenate; Sodium Selenite; Sodium Sulfate; Sodium Sulfate Anhydrous; |
| | Sodium Sulfide; Sodium Sulfite; Sodium Tartrate; Sodium |
| | Tetrachloroaurate(Iii); Sodium Tetradecyl Sulfate; Sodium Thiosulfate; Sodium |
| | Tripolyphosphate; Sodium Vanadate; Sofosbuvir; Soft Wax (Petrolatum); |
| | Solaninum; Solanum; Solanum Arrebenta; Solanum Mammosum; Solanum |
| | Nigrum; Solifenacin Succinate; Solomon's Seal; Solriamfetol; Soluble Liver |
| | Extract; Somatostatin (Somatostatin Acetate); Somatrem; Somatropin; Sonidegib |
| | Phosphate; Sorafenib (Sorafenib Tosylate); Sorbitol; Sotalol Hydrochloride; |
| | Soybean; Soybean Oil; Sparteine Sulfate; Spearmint; Spectinomycin; |
| | Spectinomycin (Spectinomycin Dihydrochloride Pentahydrate); Spectinomycin |
| | (Spectinomycin Dihydrochloride); Spectinomycin (Spectinomycin |
| | Hydrochloride); Spectinomycin (Spectinomycin Sulfate); Spectinomycin |
| | Hydrochloride; Spigelia; Spigelia Anthelmia; Spigelia Marylandica; Spilanthes |
| | Oleracea; Spinach; Spinosad; Spiramycin; Spirea Latifolia; Spironolactone; |
| | Spongia Tosta; Squalane; Squalene; Squill; Standardized Bermuda Grass Pollen; |
| | Standardized Cat Hair Acetone Precipitated; Standardized Cat Pelt; Standardized |
| | Cat Pelt Acetone Precipitated; Standardized Kentucky Blue/June Grass Pollen; |
| | Standardized Meadow Fescue Grass Pollen; Standardized Orchard Grass Pollen; |
| | Standardized Perennial Rye Grass Pollen; Standardized Redtop Grass Pollen; |
| | Standardized Short Ragweed Pollen Allergenic Extract; Standardized Sweet |
| | Vernal Grass Pollen; Standardized Timothy Grass Pollen; Stannic Iodide; |
| | Stannous Chloride; Stannous Chloride Dihydrate; Stannous Fluoride; Stannous |
| | Iodide; Stanozolol; Staphylococcus Albus; Staphylococcus Aureus; Star Anise; |
| | Starch; Stavudine; Stearic Acid; Stearic Acid (Borage Oil); Sterculia Acuminata; |
| | Sterculia Urens; Sterile Water; Sterile Water (Diluent); Sternone; Sticta |
| | Pulmonaria; Stillingia; Stiripentol; Storax; Stramonium; Strawberry; |
| | Streptococcus (Diplococcus) Pneumoniae; Streptococcus (Viridans And Non- |
| | Hemolytic); Streptococcus Antigen Group C; Streptococcus Non-Hemolytic; |
| | Streptococcus Pyogenes; Streptococcus Thermophilus; Streptococcus Viridans; |
| | Streptokinase; Streptomycin; Streptomycin (Streptomycin Sulfate); |
| | Streptomycin Sulfate; Streptozocin; Strontium Carbonate; Strontium Chloride; |
| | Strontium Chloride Sr 89; Strontium Nitrate Sr 85; Strophanthus; Strophanthus |
| | Hispidus; Strophanthus Sarmentosus; Strychnine Sulfate; Substituted Phenols; |
| | Succinic Acid; Succinylcholine Chloride; Succinylsulfathiazole; Sucralfate; |
| | Sucrose; Sufentanil (Sufentanil Citrate); Sugammadex (Sugammadex Sodium); |
| | Sulbactam (Sulbactam Benzathine); Sulesomab; Sulfabenzamide Sodium; |
| | Sulfacetamide; Sulfacetamide Sodium; Sulfadiazine; Sulfadiazine (Sulfadiazine |
| | Sodium); Sulfadimethoxine; Sulfadoxine; Sulfaguanidine; Sulfamerazine; |
| | Sulfamerazine Sodium; Sulfamethazine; Sulfamethazine Bisulfate; |
| | Sulfamethazine Sodium; Sulfamethoxazole; Sulfanilamide; |
| | Sulfanilylbenzamide; Sulfapyridine; Sulfaquinoxaline; Sulfaquinoxaline |
| | (Sulfaquinoxaline Sodium); Sulfasalazine; Sulfate (Sodium Sulfate); Sulfated |
| | Oils; Sulfathiazole; Sulfathiazole (Sulfathiazole Sodium); Sulfathiazole Sodium; |
| | Sulfathiazole Sodium (Sulfathiazole Sodium Sesquihydrate); Sulfinpyrazone; |
| | Sulfisoxazole; Sulfisoxazole (Sulfisoxazole Acetyl); Sulfur; Sulfur (Sodium |
| | Thiosulfate); Sulfur (Sodium Thiosulphate); Sulfur (Sulfoxide); Sulfur (Sulfur); |
| | Sulfur Hexafluoride; Sulfur Iodide; Sulfurated Potash; Sulfuric Acid; Sulfurous |
| | Acid; Sulindac; Sulisobenzone; Sumach; Sumatriptan (Sumatriptan |
| | Hemisulfate); Sumatriptan (Sumatriptan Succinate); Sumbul; Sunflower; |
| | Sunitinib (Sunitinib Malate); Sunitinib Malate; Surfactant-Associated Proteins |
| | Sp-B And Sp-C; Sutilains; Suvorexant; Sweet Vernal Grass; Sycamore; |
| | Symclosene; Symphoricarpus Racemosus |
| T | Tacrolimus; Tacrolimus (Tacrolimus Monohydrate); Tadalafil; Tafluprost; Talc; |
| | Taliglucerase Alfa; Tallow; Tamarinds; Tamoxifen (Tamoxifen Citrate); |
| | Tamoxifen Citrate; Tamsulosin Hydrochloride; Tanacetum Vulgare; Tannic |
| | Acid; Tapentadol (Tapentadol Hydrochloride); Tapentadol Hydrochloride; Tar |
| | (Polytar); Taraxacum Officinale Weber; Tarentula Cubensis; Tarentula Hispana; |
| | Tartaric Acid; Taurine; Tauroselcholic Acid (75se); Taxus Baccata; Tazarotene; |
| | Tazobactam (Tazobactam Sodium); Tea Tree Oil; Technetium Tc 99m; |
| | Technetium Tc 99m Red Blood Cells; Tedizolid Phosphate; Teduglutide; |
| | Teduglutide Recombinant; Teflubenzuron; Telaprevir; Telavancin; Telavancin |
| | (Telavancin Hydrochloride); Telavancin Hydrochloride; Telbivudine; |
| | Telithromycin; Tellurium Metallicum; Telmisartan; Telmisartan (Telmisartan |
| | Sodium); Telotristat Ethyl (Telotristat Etiprate); Temazepam; Temozolomide; |
| | Temsirolimus; Tenecteplase; Teniposide; Tenofovir Alafenamide (Tenofovir |
| | Alafenamide Hemifumarate); Tenofovir Disoproxil Fumarate; Tenoxicam; |
| | Terazosin (Terazosin Hydrochloride); Terazosin Hydrochloride; Terbinafine; |
| | Terbinafine (Terbinafine Hydrochloride); Terbinafine Hydrochloride; |
| | Terbutaline Sulfate; Terconazole; Terephthalylidene Dicamphor Sulfonic Acid; |
| | Terfenadine; Teriflunomide; Teriparatide; Teriparatide Recombinant Human; |
| | Terpin Hydrate; Tesamorelin (Tesamorelin Acetate); Testosterone; Testosterone |
| | Cypionate; Testosterone Enanthate; Testosterone Enanthate Benzilic Acid |
| | Hydrazone; Testosterone Propionate; Testosterone Undecanoate; Tetanus |
| | Immunoglobulin (Human); Tetanus Protein; Tetanus Toxoid; Tetanus Toxoid |
| | Adsorbed; Tetanus Toxoid Antigen; Tetrabenazine; Tetracaine; Tetracaine |
| | (Tetracaine Hydrochloride); Tetracaine Hydrochloride; Tetracosactide; |
| | Tetracosactide (Tetracosactide Zinc Hydroxide); Tetracycline (Base) |
| | (Tetracycline Hydrochloride); Tetracycline Hydrochloride; Tetradymite; |
| | Tetrahydrofurfuryl Alcohol; Tetrahydrozoline Hydrochloride; Tetrakis |
| | [Cu(Mibi)4]Bf4; Tetramethylthiuram Monosulfide; Tetramisole Hydrochloride; |
| | Tetrapotassium Ethylenediamine Tetraacetate; Tetrapotassium Pyrophosphate; |
| | Tetrasodium Pyrophosphate; Tetrofosmin; Teucrium Scorodonia; Tezacaftor; |
| | Thalamus; Thalidomide; Thallium Aceticum Homeo; Thallium Aceticum |
| | Homeo Chemical; Thallium Chloride(Ti 201); Thallium Metallicum; Thallium |
| | Sulfate; Thaspium Aureum; Thea Sinensis; Thenyldiamine Hydrochloride; |
| | Theophylline; Theridion; Thiabendazole; Thiacetarsamide Sodium; Thiamazole; |
| | Thiamine Hydrochloride; Thiamine Mononitrate; Thiamylal Sodium; |
| | Thimerosal; Thioctic Acid; Thioguanine; Thiopental Sodium; Thioproperazine |
| | (Thioproperazine Mesylate); Thioridazine; Thioridazine Hydrochloride; |
| | Thiosinamine; Thiostrepton (Antibiotic Of A Streptomyces Species); Thiotepa; |
| | Thiothixene; Thiram; Threonine; Thrombin; Thrombin (Bovine); Thrombin |
| | (Human); Thrombin Alfa; Thuja Lobbi; Thuja Occidentalis; Thyme; Thymol; |
| | Thymol (Thyme Oil); Thymol Iodide; Thymus Tissue Glands; Thyroid; |
| | Thyrotropin Alfa; Tiagabine Hydrochloride; Tiamulin (Tiamulin Fumarate); |
| | Tiamulin (Tiamulin Hydrogen Fumarate); Tiamulin Fumarate; Tiaprofenic Acid; |
| | Tibolone; Ticagrelor; Ticarcillin (Ticarcillin Disodium); Ticarcillin Disodium; |
| | Tick-Borne Encephalitis Vaccine (Inactivated); Ticlopidine Hydrochloride; |
| | Tigecycline; Tiger Lily; Tildipirosin; Tildrakizumab-Asmn; Tilia Tomentosa; |
| | Tillandsia; Tilmicosin; Tilmicosin (Tilmicosin Phosphate); Tiludronic Acid |
| | (Tiludronate Disodium); Timolol (Timolol Maleate); Timolol Maleate; Tin; Tin |
| | (Stannous Chloride); Tinzaparin Sodium; Tioconazole; Tiotropium (Tiotropium |
| | Bromide Monohydrate); Tipiracil (Tipiracil Hydrochloride); Tipranavir; |
| | Tirofiban (Tirofiban Hydrochloride); Tisagenlecleucel; Titanium Dioxide; |
| | Titanium Dioxide Coated Mica; Titanium Metallicum; Tixocortol Pivalate; |
| | Tizanidine (Tizanidine Hydrochloride); Tobacco; Tobramycin; Tobramycin |
| | (Tobramycin Sulfate); Tobramycin Sulfate; Tocainide Hydrochloride; Toceranib |
| | (Toceranib Phosphate); Tocilizumab; Tocophersolan; Tofacitinib (Tofacitinib |
| | Citrate); Tofacitinib Citrate; Tolazoline (Tolazoline Hydrochloride); |
| | Tolbutamide; Tolcapone; Tolfenamic Acid; Tolmetin (Tolmetin Sodium); |
| | Tolmetin Sodium; Tolnaftate; Tolterodine Tartrate; Toltrazuril; Tolvaptan; |
| | Tomato; Tonka Bean; Topiramate; Topotecan (Topotecan Hydrochloride); |
| | Tormentilla; Torsemide; Tositumomab; Total Protein; Toxicophis Pugnax; |
| | Trabectedin; Tradescantia Diuretica; Tramadol Hydrochloride; Trametinib; |
| | Trandolapril; Tranexamic Acid; Tranylcypromine (Tranylcypromine Sulfate); |
| | Tranylcypromine Sulfate; Trastuzumab; Trastuzumab Emtansine; Travoprost; |
| | Trazodone (Trazodone Hydrochloride); Trazodone Hydrochloride; Tree Of |
| | Heaven; Trenbolone Acetate; Treprostinil (Treprostinil Sodium); Tretinoin; |
| | Triamcinolone; Triamcinolone Acetonide; Triamcinolone Diacetate; |
| | Triamcinolone Hexacetonide; Triamterene; Triazolam; Tribromsalan; Tricaine; |
| | Trichlorfon; Trichlormethiazide; Trichlorofluoromethane; Trichophyton |
| | Mentagrophytes Antigen; Triclocarban; Triclosan; Triethanolamine Polypeptide |
| | Cocoate Condensate; Triethanolamine Polypeptide Oleate Condensate; |
| | Triethanolamine Stearate; Triethylene Glycol; Trifluoperazine (Trifluoperazine |
| | Hydrochloride); Trifluoperazine Hydrochloride; Trifluridine; Trifolium; |
| | Trifolium Pratense; Trifolium Repens; Trihexyphenidyl Hydrochloride; Trillium |
| | Pendulum; Trilostane; Trimebutine Maleate; Trimeprazine (Trimeprazine |
| | Tartrate); Trimeprazine Tartrate; Trimethadione; Trimethoprim; Trimethoprim |
| | (Sulfadiazine Sodium); Trimethoprim (Trimethoprim Sulfate); |
| | Trimethylaminum; Trimetrexate (Trimetrexate Glucuronate); Trimipramine |
| | (Trimipramine Maleate); Trimipramine Maleate; Triosteum Perfoliatum; |
| | Trioxsalen; Tripelennamine Hydrochloride; Triprolidine Hydrochloride; |
| | Triptorelin (Triptorelin Acetate); Triptorelin (Triptorelin Pamoate); Triptorelin |
| | Acetate; Triptorelin Pamoate; Tris Hydroxymethyl Nitromethane; Trolamine; |
| | Trolamine Salicylate; Trometamol; Tropaeolum Majus; Tropicamide; Trospium |
| | Chloride; Trovafloxacin (Alatrofloxacin Mesylate); Trovafloxacin |
| | (Trovafloxacin Mesylate); Trumpet Weed; Trypan Blue; Tryptophan; Tuberculin |
| | Old; Tubocurarine Chloride; Tulathromycin; Turmeric; Turnip; Turoctocog Alfa; |
| | Turpentine; Tussilago Fragrans; Tussilago Petasites; Tylosin; Tylosin (Tylosin |
| | Phosphate); Tylosin (Tylosin Tartrate); Tylosin Phosphate; Tylosin Tartrate; |
| | Tylvalosin (Tylvalosin Tartrate); Typhoid Vaccine; Typhoid Vaccine |
| | (Salmonella Typhi Ty21a); Tyrosine; Tyrothricin |
| U | Ubidecarenone; Ubidecarenone (Coenzyme Q10); Ubidecarenone (Ubiquinone- |
| | 10); Ulex Europaeus; Ulipristal Acetate; Ulmus Procera Flos; Umeclidinium |
| | (Umeclidinium Bromide); Umeclidinium Bromide: Vilanterol Trifenatate; |
| | Undecenoylcollagenic Acid; Undecylenic Acid; Unoprostone Isopropyl; |
| | Uranium Nitrate; Urea; Urea Hydrogen Peroxide; Uric Acid; Urofollitropin; |
| | Urokinase; Ursodiol; Urtica Dioica; Urtica Urens; Usnea Barbata; Ustekinumab; |
| | Ustilago Maydis; Uterus Tissue; Uva Ursi |
| V | Vaccinium Macrocarpon; Vaccinium Myrtillus; Valacyclovir (Valacyclovir |
| | Hydrochloride Monohydrate); Valacyclovir (Valacyclovir Hydrochloride); |
| | Valerian; Valerian (Valeriana Officinalis); Valganciclovir (Valganciclovir |
| | Hydrochloride); Valine; Valproic Acid; Valproic Acid (Divalproex Sodium); |
| | Valproic Acid (Sodium Valproate); Valrubicin; Valsartan; Valsartan (Sacubitril |
| | Valsartan Sodium Hydrate Complex); Vanadium; Vanadium (Sodium |
| | Metavanadate); Vanadium (Sodium Vanadate); Vanadium (Vanadium Citrate; |
| | Vanadium (Vanadium Citrate); Vanadium (Vanadium Hvp Chelate); Vanadium |
| | (Vanadium Organic Complex); Vanadium (Vanadium Pentoxide); Vanadium |
| | (Vanadium Proteinate); Vanadium (Vanadium Sulfate); Vanadium Hvp Chelate; |
| | Vanadium Pentoxide; Vanadium Proteinate; Vancomycin (Vancomycin |
| | Hydrochloride); Vancomycin Hydrochloride; Vandetanib; Vanillylnonamide; |
| | Vardenafil (Vardenafil Hydrochloride Trihydrate); Vardenafil (Vardenafil |
| | Hydrochloride); Varenicline (Varenicline Tartrate); Varicella Zoster |
| | Immunoglobulin (Human); Varicella-Zoster Virus Glycoprotein E Antigen; |
| | Varicella-Zoster Virus Vaccine Live Attenuated (Oka/Merck Strain); |
| | Vasopressin; Vasopressin (Arginine Vasopressin); Vecuronium Bromide; |
| | Vedaprofen; Vedolizumab; Velaglucerase Alfa; Velpatasvir; Vemurafenib; |
| | Venetoclax; Venlafaxine (Venlafaxine Hydrochloride); Venlafaxine |
| | Hydrochloride; Verapamil Hydrochloride; Verbascum Thapsus; Verbena |
| | Hastata; Verbena Officinalis; Vernakalant Hydrochloride; Veronica Beccabunga; |
| | Veronica Officinalis; Vertebra; Verteporfin; Vesicaria; Vespa Crabro; Vespula |
| | Spp Venom Protein Extract; Vibrio Cholerae Inaba 48 Classical Biotype; Vibrio |
| | Cholerae Inaba 6973 E1 Tor Biotype; Vibrio Cholerae Ogawa 50 Classical |
| | Biotype; Viburnum; Viburnum Lantana; Viburnum Opulus; Viburnum |
| | Prunifolium; Vigabatrin; Vilanterol (Vilanterol Trifenatate); Vilazodone |
| | Hydrochloride; Vinblastine Sulfate; Vincristine Sulfate; Vindesine Sulfate; |
| | Vinegar; Vinorelbine (Vinorelbine Tartrate); Viola Odorata; Viola Tricolor; |
| | Virginiamycin; Virola Sebifera; Vismodegib; Vitamin A; Vitamin A (Acetate); |
| | Vitamin A (Beta-Carotene); Vitamin A (Cod Liver Oil); Vitamin A (Dunaliella |
| | Salina; Vitamin A (Fish Liver Oil; Vitamin A (Fish Liver Oil); Vitamin A (Fish |
| | Oil); Vitamin A (Halibut Liver Oil); Vitamin A (Lemon Grass); Vitamin A |
| | (Retinol Palmitate; Vitamin A (Retinol Palmitate); Vitamin A (Retinol); Vitamin |
| | A (Retinyl Palmitate); Vitamin A (Retinyl Propionate); Vitamin A (Rosin Oil); |
| | Vitamin A (Shark Liver Oil); Vitamin A (Sodium Selenite); Vitamin A (Vitamin |
| | A Acetate; Vitamin A (Vitamin A Acetate); Vitamin A (Vitamin A Propionate); |
| | Vitamin A Acetate; Vitamin A Propionate; Vitamin B1; Vitamin B1 |
| | (Acetiamine); Vitamin B1 (Thiamine Diphosphate); Vitamin B1 (Thiamine |
| | Hydrochloride); Vitamin B1 (Thiamine Mononitrate; Vitamin B1 (Thiamine |
| | Mononitrate); Vitamin B1 (Thiamine); Vitamin B1 (Vitamin B1); Vitamin B1 |
| | (Yeast); Vitamin B12; Vitamin B12 (Cobalamin); Vitamin B12 (Cobamamide; |
| | Vitamin B12 (Cobamamide); Vitamin B12 (Cyanocobalamin; Vitamin B12 |
| | (Cyanocobalamin); Vitamin B12 (Hydroxocobalamin; Vitamin B12 |
| | (Hydroxocobalamin); Vitamin B12 (Methylcobalamin); Vitamin B12 (Vitamin |
| | B12); Vitamin B12 (Yeast); Vitamin B2; Vitamin B2 (Riboflavin; Vitamin B2 |
| | (Riboflavin 5′-Phosphate Sodium; Vitamin B2 (Riboflavin 5′-Phosphate |
| | Sodium); Vitamin B2 (Riboflavin Hydrochloride; Vitamin B2 (Riboflavin |
| | Sodium Phosphate); Vitamin B2 (Riboflavin); Vitamin B2 (Riboflavin-5- |
| | Phosphate; Vitamin B2 (Riboflavin-5-Phosphate); Vitamin B2 (Vitamin B2); |
| | Vitamin B2 (Yeast); Vitamin B3 (Nicotinamide); Vitamin B3 (Nicotinic Acid); |
| | Vitamin B6; Vitamin B6 (Pyridoxal-5-Phosphate; Vitamin B6 (Pyridoxal-5- |
| | Phosphate); Vitamin B6 (Pyridoxine 5′ Phosphate); Vitamin B6 (Pyridoxine |
| | Hydrochloride; Vitamin B6 (Pyridoxine Hydrochloride); Vitamin B6 |
| | (Pyridoxine); Vitamin B6 (Pyridoxol Hydrochloride); Vitamin B6 (Yeast); |
| | Vitamin C; Vitamin C (Acerola; Vitamin C (Acerola); Vitamin C (Ascorbic Acid; |
| | Vitamin C (Ascorbic Acid); Vitamin C (Calcium Ascorbate; Vitamin C (Calcium |
| | Ascorbate); Vitamin C (Dextrose); Vitamin C (Dog Rose; Vitamin C |
| | (Magnesium Ascorbate; Vitamin C (Magnesium Ascorbate); Vitamin C |
| | (Niacinamide Ascorbate; Vitamin C (Niacinamide Ascorbate); Vitamin C |
| | (Nicotinamide Ascorbate); Vitamin C (Potassium Ascorbate); Vitamin C (Rose |
| | Hips; Vitamin C (Rose Hips); Vitamin C (Sago Palm); Vitamin C (Sodium |
| | Ascorbate; Vitamin C (Sodium Ascorbate); Vitamin C (Vitamin C; Vitamin C |
| | (Vitamin C); Vitamin C (Zinc Ascorbate; Vitamin C (Zinc Ascorbate); Vitamin |
| | D; Vitamin D (Calciferol); Vitamin D (Cholecalciferol); Vitamin D (Cod Liver |
| | Oil); Vitamin D (Dexpanthenol); Vitamin D (Ergocalciferol); Vitamin D (Fish |
| | Liver Oil); Vitamin D (Fish Oil); Vitamin D (Halibut Liver Oil); Vitamin D |
| | (Magnesium Carbonate); Vitamin D (Vitamin D Acetate); Vitamin D (Vitamin |
| | D2); Vitamin D (Vitamin D3); Vitamin D2; Vitamin D2 (Ergocalciferol); |
| | Vitamin D2 (Ergosterol); Vitamin D2 (Vitamin D2); Vitamin D3; Vitamin D3 |
| | (Cholecalciferol); Vitamin D3 (Fish Liver Oil); Vitamin D3 (Halibut Liver Oil); |
| | Vitamin D3 (Vitamin D3); Vitamin E; Vitamin E (Acetate); Vitamin E (Alpha |
| | Tocopherol Acetate); Vitamin E (Alpha Tocopherol); Vitamin E (D-Alpha |
| | Tocopherol; Vitamin E (D-Alpha Tocopherol); Vitamin E (D-Alpha Tocopheryl |
| | Acetate; Vitamin E (D-Alpha Tocopheryl Acetate); Vitamin E (D-Alpha |
| | Tocopheryl Acid Succinate; Vitamin E (D-Alpha Tocopheryl Acid Succinate); |
| | Vitamin E (D-Alpha Tocopheryl); Vitamin E (Dl-Alpha Tocopherol; Vitamin E |
| | (Dl-Alpha Tocopherol); Vitamin E (Dl-Alpha Tocopheryl Acetate; Vitamin E |
| | (Dl-Alpha Tocopheryl Acetate); Vitamin E (Dl-Alpha Tocopheryl Acid |
| | Succinate); Vitamin E (Fish Oil); Vitamin E (Mixed Tocopherols; Vitamin E |
| | (Mixed Tocopherols); Vitamin E (Soybean Oil; Vitamin E (Soybean Oil); |
| | Vitamin E (Vegetable Oils); Vitamin E (Vitamin E Acetate); Vitamin E (Vitamin |
| | E); Vitamin K (Menadione Sodium Bisulfite); Vitamin K1; Vitex Agnus Castus; |
| | Vitis Vinifera; Von Willebrand Factor (Human); Von Willebrand Factor |
| | (Recombinant); Vorapaxar Sulfate; Voriconazole; Vorinostat; Vortioxetine |
| | (Vortioxetine Hydrobromide); Vortioxetine Hydrobromide; Voxilaprevir |
| W | Wall Pellitory; Wallflower; Warfarin Sodium; Wasp; Wasp Venom Protein; |
| | Water; Water (Diluent); Water Cress; Water Hemlock; Water Lily; Watermelon; |
| | Western Ragweed; Wheat; Wheat Germ (Wheat); Wheat Germ Oil; White Cedar |
| | Oil; White Faced Hornet Venom Protein; White Hellebore Root; White |
| | Mulberry; White Petrolatum; White Pine; Wiesbaden; Wild Cherry; Wild |
| | Lettuce; Wild Radish; Wild Thyme; Willow; Willow Bark; Wine; Winter Cherry; |
| | Wood Betony; Woodruff Herb; Wool Alcohols; Wyethia Helenioides |
| X | X-Ray; Xanthinol Niacinate; Xanthoxylum; Xenon 133 Xe; Xylazine (Xylazine |
| | Hydrochloride); Xylazine Hydrochloride; Xylitol; Xylometazoline |
| | Hydrochloride; Xylose |
| Y | Yeast; Yellow Dock; Yellow Fever Vaccine; Yellow Hornet Venom Protein; |
| | Yellow Jacket Venom Protein; Yellow Phenolphthalein; Yohimbine; Yohimbine |
| | (Yohimbine Hydrochloride); Yohimbine Hydrochloride; Yttrium-90 Chloride; |
| | Yucca Filamentosa |
| Z | Zafirlukast; Zalcitabine; Zaleplon; Zanamivir; Zea Mays; Zeranol; Zidovudine; |
| | Zilpaterol Hydrochloride; Zinc; Zinc (Sea Proteinate); Zinc (Zinc Acetate); Zinc |
| | (Zinc Amino Acid Chelate); Zinc (Zinc Ascorbate); Zinc (Zinc Chelate); Zinc |
| | (Zinc Chloride); Zinc (Zinc Citrate; Zinc (Zinc Citrate); Zinc (Zinc Diammonium |
| | Edta); Zinc (Zinc Fumarate; Zinc (Zinc Fumarate); Zinc (Zinc Gluconate; Zinc |
| | (Zinc Gluconate); Zinc (Zinc Glutarate); Zinc (Zinc Hvp Chelate; Zinc (Zinc Hvp |
| | Chelate); Zinc (Zinc Malate); Zinc (Zinc Maleate); Zinc (Zinc Organic |
| | Complex); Zinc (Zinc Oxide; Zinc (Zinc Oxide); Zinc (Zinc Proteinate; Zinc |
| | (Zinc Proteinate); Zinc (Zinc Succinate; Zinc (Zinc Succinate); Zinc (Zinc |
| | Sulfate; Zinc (Zinc Sulfate); Zinc Acetate; Zinc Aspartate; Zinc Carbonate; Zinc |
| | Chloride; Zinc Citrate; Zinc Cyanide; Zinc Dibutyldithiocarbamate; Zinc |
| | Diethyldithiocarbamate; Zinc Gluconate; Zinc Hvp Chelate; Zinc Hvp Chelate |
| | (Zinc Gluconate); Zinc Iodide; Zinc Oxide; Zinc Oxide (Zinc Oxide); Zinc |
| | Peroxide; Zinc Phenol sulphonate; Zinc Phosphate; Zinc Phosphide; Zinc |
| | Proteinate; Zinc Sulfate; Zinc Sulfate (Zinc Sulfate Monohydrate); Zinc Sulfate |
| | (Zinc Sulfate); Zinc Sulfate Heptahydrate; Zinc Sulfate Monohydrate; Zinc |
| | Undecylenate; Zinc Valerate; Zincum Bromatum; Zincum Picricum; Ziprasidone |
| | (Ziprasidone Hydrochloride Monohydrate); Zirconium Hydroxychloride; |
| | Zizyphus Spinosa; Zoledronic Acid; Zoledronic Acid (Zoledronic Acid |
| | Hemipentahydrate); Zoledronic Acid (Zoledronic Acid Monohydrate); |
| | Zolmitriptan; Zolpidem Tartrate; Zonisamide; Zopiclone; Zucapsaicin; |
| | Zuclopenthixol (Zuclopenthixol Hydrochloride); Zuclopenthixol Acetate; |
| | Zuclopenthixol Decanoate |

It should be understood that certain of the listed materials may be used for topical application of vapor (for example, capsasin should not be used for inhalation but may provide a benefit if applied topically as a vapor). Some of the listed materials may be appropriate for topical, inhalation, and/or one or more additional modes of application.

While there are a variety of pharmaceutical uses, examples may be helpful. In one application, following a cardiac event or stroke, it is desirable to have aspirin while awaiting medical care. However, an aspirin ingested by oral route may take 20 or 30 minutes before reaching a desirable point of efficacy. In an urgent situation where blood thinning is critical, the risks of inhaling vaporized aspirin may be outweighed by the benefits of more rapid uptake. In such a case, aspirin may be vaporized using one or more of the inventions herein. Similarly, erectile dysfunction drugs are commonly slow to reach efficacy. The most rapidly efficacious of the popular drugs within this class of drugs is thought to be Sildenafil, or Viagra®. With an onset time of approximately 20 minutes, for many “the mood” will have passed before the medication becomes effective. By vaporizing and inhaling such medication, efficacy should be reached an order of magnitude more quickly. In one implementation, the device may be operably connected to a communication modality, such as a wide area network or a phone, and send a request for help upon actuation of the device.

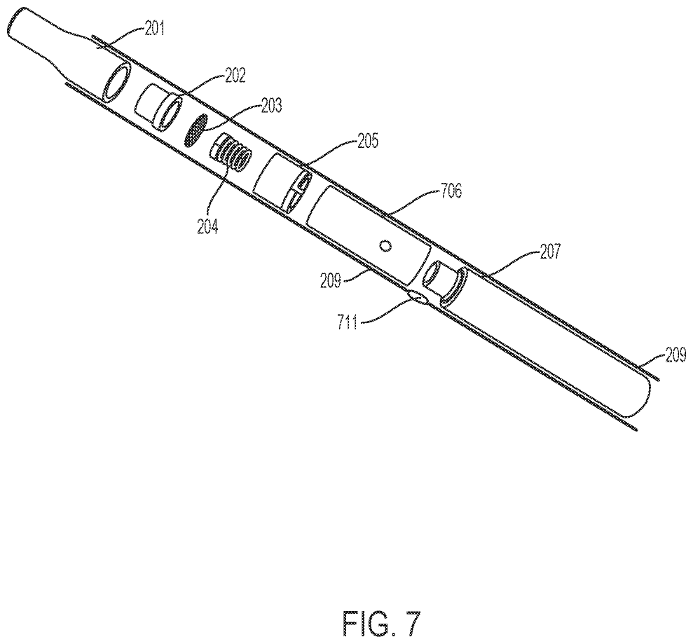

Referring to FIG. 7, therein is shown a diagram of a self-contained medication vaporizer. The medication vaporizer of FIG. 7 is similar in structure to the vaporizer structure described in relation to FIG. 2, except that the vaporizer of FIG. 2 contains an external source of non-oxygenated gas, whereas the non-oxygenated gas source of vaporizer of FIG. 7 is self-contained within the vaporization chamber 706.

201-205, 207 and 209 are as described in FIG. 2 (see paras. [0036]-[0040], [0042] and [0044] above).

706 is a self-contained heating or vaporization chamber, comprising a non-oxygenated gas source contained within the chamber.

711 is a gas inlet for replacement gas, to allow the replacement gas to flow into the vaporizer to avoid creating a vacuum as the vaporized elements are inhaled through mouth piece 201. In one aspect, the replacement gas may be a non-oxygenated replacement gas.

In an embodiment, an apparatus for rapidly administering a drug comprises a chamber containing non-oxygenated gas and an amount of the drug, the chamber operably coupled to a heat source, wherein heat is applied from the heat source upon actuation of the apparatus until a boiling temperature of the drug is reached or exceeded, and wherein when the apparatus provides an indication that sufficient drug has been vaporized, the apparatus ceases heating, and a tube or other conduit to allow inhalation of the gas and vaporized drug from the apparatus.

In some aspects, the drug is a blood thinner, salicylic acid, or an erectile dysfunction drug. In other aspects, the drug may be naloxone or a benzodiazepine.

The apparatus may also be operably connected to a communication modality and upon actuation of the apparatus, the apparatus actuates the communication modality and requests assistance. In addition, flavor and smell sources may be added. For example, a person on a diet may desire a chocolate flavor. Various flavors, such as chocolate/cocoa bean, vanilla, coffee, and others may be incorporated into the mixture before vaporizing. Flavor and smell sources may be utilized independently as well. In addition, caffeine may be vaporized.

The boiling point of caffeine is around 178 degrees C.

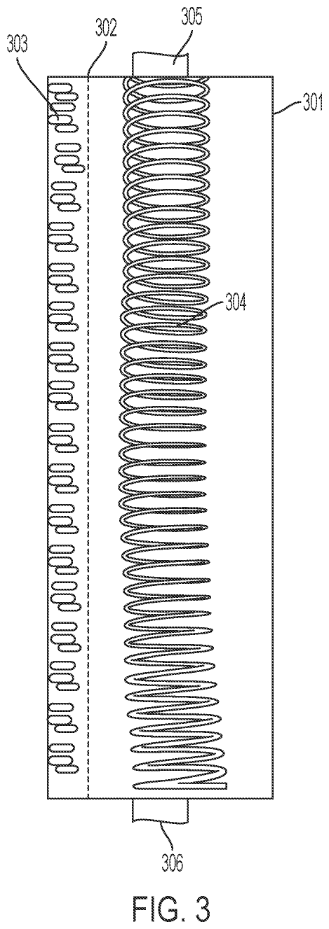

Turning to FIG. 3:

301 is a cartridge.

302 is an optional element to hold the material to be vaporized 303 in place.

303 is the material to be vaporized.

304 is a resistance or other heating element.

305 is a connector (terminal) to allow power to be provided to the heating element.

306 is a second connector (terminal) to allow power to be provided to the heating element.

In another aspect, the material to be vaporized may be pre-packaged in a disposable or reusable container that can be inserted into the vaporization chamber (a “cartridge”) 301. A cartridge may be labelled with the ingredients and/or labelled with vaporization efficacy. In one aspect, the data is incorporated into a memory contained within the cartridge. The memory may be electronic or may take the form of something that may be read by a machine, such as a bar code or QR code. In one aspect, the vaporizer may be programmed to measure the flow of gas over the material to be vaporized and the temperature at which the gas is flowing in order to calculate the likely contents of the vapor. For example, if 250 mg of cannabis contains 50 mg of THC, at a flow rate of X, with the temperature moving linearly from A to B, and stopping after Y seconds, the device may reference tables (which may be contained in the device, downloaded, be present in the cartridge, or otherwise) to determine how much THC has been delivered. In addition, or in the alternative, chromatography may be utilized in conjunction with flow rate to determine the amount of THC that has been delivered. In another aspect, the full cartridge may be vaporized into a container, such as a balloon or bag. The device would indicate how much THC is estimated to be present in the container. The user could then “ballpark” how much of the container to consume to reach the desired level of THC. Alternatively, the container may be connected to a metering device, or discharged in part into a smaller container of a known volume. In one simple implementation, the measured percentage of THC (and THCA) by weight may be added to the estimated percentage by weight of compounds that will boil off at the temperature being used. The percentage by weight of compounds expected to boil off may then be multiplied by the weight of the plant matter prior to heating. Vaporization would be considered complete, and in one aspect the heating or other elements deactivated or modified, as the weight of the remaining plant matter nears and/or reaches the expected weight post-boil-off. As the device would have calculated the volume of gas in the container, it would be possible to determine how much THC is delivered by each metered dose. For example, if 10 cubic centimeters of gas is present in the container, each cubic centimeter of gas delivered by the metered device would be 10% of the total THC content in the container. It is important to note that while we discuss this in the context of cartridges, it may also be utilized in cartridge-free configurations.

In one aspect, the cartridges may be accompanied by an amount of a known gas. For example, if it is known that the cannabis in cartridge A will require 25 cubic centimeters of gas to vaporize the THC, cartridge A may have a chamber filled with 25 (or more) cubic centimeters of compressed gas. Alternatively, the compressed gas may be separate (either as a multi-use tank of gas or a small, single-use tank). The label on the cartridge would indicate how much gas was required to complete the vaporization, and the device would deliver that much gas. In one aspect, a device capable of puncturing the cartridge is actuated. In another aspect, the puncturing device is actuated in combination with, or in preparation for, inhalation of the contents of the chamber. A second puncture may be made so that the gas can flow out of the cartridge. A regulator may be used to change the outflow pressure from a cartridge where the pressure within the cartridge is different than the ambient pressure.

In another aspect, the cartridges may have a heating element contained internally 304. For example, KANTHAL® or Nickel-Chromium wire may be run (coiled or not) through the center of the cartridge. In such a case, concerns about replacing heating elements are eliminated, as a fresh heating element accompanies each cartridge. In another aspect, reuse of the cartridge may be precluded by sending an electrical charge down the heating element after the vaporization is complete, where the electrical charge is sufficient to render the heating element non-functional. In the resistance heating variant, this may involve a charge sufficient to cause the heating element to break so that it can no longer carry a current through the length of the cartridge.

Generally, an apparatus for vaporizing plant matter, may comprise, (i) a cartridge-receiving fitting, (ii) a cartridge containing plant matter for vaporization, (iii) a heating element internal or external to the cartridge, the cartridge filled with a gas having an amount of oxygen less than a LOC for combustion of the plant materials, and (iv) a gas-bearing connector capable of connecting to the cartridge-receiving fitting, thereby allowing the gas to be inhaled.

The apparatus may further comprise a resistance heating element contained within the cartridge and a power source for the resistance heating element. Additionally, the apparatus may further comprise a penetrating device to puncture one or more surfaces of the cartridge and/or a conduit for transport of contents of the cartridge for inhalation. In some aspects the apparatus may comprise a penetrating device to puncture a portion of the cartridge operably connected to the conduit

It should be understood that a cartridge bearing compressed gas as well as plant matter 303 may be used as the heating chamber itself, whether the heat is internally supplied 304 (such as via a KANTHAL® element) or externally supplied. If externally supplied, one mechanism is to have an effective heat-conductive substance, such as copper, form at least one wall or a portion of a wall of the cartridge. In one aspect, the heat conducting substance may extend into the chamber. If internally supplied, power may be provided through terminals 305, 306 on the cartridge 301.

In another aspect, the vapor may be discharged into a container that is lined with oleophobic material, hydrophobic material, and/or a water-based lubricant in order to impede the speed with which the materials in the vapor precipitate out and affix to the walls of the container.

In another aspect, materials may have different boiling points. For example, Aspirin's boiling point is approximately 140 degrees C., while Codeine's boiling point is approximately 250 degrees C. In such a case, where it is desirable to separate the aspirin from the codeine, it may be appropriate to heat the combination to 240 degrees C., causing the Aspirin to boil off and leave only the Codeine (for Aspirin with Codeine and Caffeine, the 240 degree mark exceeds the 178 degree boiling point of caffeine). Because the temperature does not reach the temperature required to fully break down the structure of a pill, it may be preferable to reduce the compound to very small pieces and/or to leave the compound in the heat for a long period of time. Without limiting the foregoing, one range of particle size may be between 3 and 100 microns. Another range would be from 100 microns to 1 mm. While it is often preferable to heat such material in a non-oxygenated atmosphere, the presence of oxygen may sometimes be acceptable, particularly where the heating chamber is not heated above combustion temperature.

With regard to heating cannabis to vaporize THC, using a non-oxygenated atmosphere with a maximum temperature of between 300 and 400 degrees C. may be desirable. We note that this temperature may be exceeded, and such excess may be desirable in order to break down plant matter trapping THC. It should also be noted that at temperatures nearing 400 degrees C., in many cases the plant matter is fully vaporized, leaving only a carbon shell. This makes it possible to vaporize a tobacco leaf, a cannabis flower, or other materials without drying them and/or without crushing or grinding them.

In another aspect, a weight measurement device may be associated with the contents of the vaporization chamber. The device would cease attempting to extract further materials from the vaporization chamber when the weight of the materials in the chamber stabilizes. That is, once the materials that can be vaporized have been vaporized, the weight should stabilize, and the device may utilize that property to determine when to cease attempting to vaporize materials.

It should be noted that different gases have different temperature conductivity characteristics. For example, nitrogen has a thermal conductivity of 0.026 W/mK at 300 degrees K (i.e., room temperature, approx. 26.8 degrees C.). Under the same conditions, Argon has a thermal conductivity of 0.018; atmospheric air has a thermal conductivity of 0.026, and helium has a thermal conductivity of 0.182. The general rule is that gas thermal conductivity decreases with increases in molecular weight. As a result, it is desirable to utilize light gases in order to better conduct heat from the heat source to the matter the user wishes to vaporize.

In one aspect, a light gas, such as helium, is utilized at the outset in order to spread the heat throughout the material as quickly as possible. Once the desired temperature is reached, the light gas would be replaced with a heavier gas, such as argon (or even nitrogen). With a constant gas flow over the material to be vaporized, such a switch of gases allows the use of less power to reach and maintain the desired temperature. To quickly cool the material, it may further be desirable to switch back to a light gas. While we discuss this in the context of a vaporizer, it should be noted that the same properties may be utilized in other applications.

In one set of implementations, one or more of atmospheric air, substantially pure oxygen (preferably in the O₂ form), or a gas mixture containing oxygen, such as a helium/oxygen mixture, may be introduced into the post-heating vapor. For example, using nitrogen gas as the non-oxygenated gas to heat the target material in, it is desirable to add enough oxygen to prevent or delay hypoxia. In one case, approximately 21 units of oxygen may be added for every 79 units of non-oxygenated gas, resulting in an end result where the gas inhaled by the user has approximately the same level of oxygen as regular atmospheric air. Similarly, atmospheric air may be added. In a preferred mode, atmospheric air is added in at least a 1 to 1 ratio, and as high as a 9 parts air to 1-part non-oxygenated gas ratio. Of course, a ratio lower than 1 to 1 may also be used if the increased risk of hypoxia is deemed acceptable.

In terms of flow rate, using nitrogen, a preferred flow rate is 8 liters per minute using a heating chamber with a capacity of 50 ml to 100 ml. A faster flow rate, such as 9, 10, 11, 12, 13, 14, 15, 16, 17, 18, 19 or 20 liters per minute results in a less dense vapor that is less likely to cause respiratory irritation. A slower flow rate uses the nonoxygenated gas more efficiently and results in a higher density vapor. A flow rate of 1, 2, 3, 4, 5, 6 or 7 liters per minute may be optimal in such a case. Thus flow rates may range anywhere between 0.1 liters per minute and 20.0 liters per minute. After the heating chamber is cleared of either all, substantially all, or enough oxygen to avoid combustion and/or oxidation, use of a valve system to prevent backflow of oxygenated gas (or simply closing the chamber) permits heating with a very small amount of non-oxygenated gas.

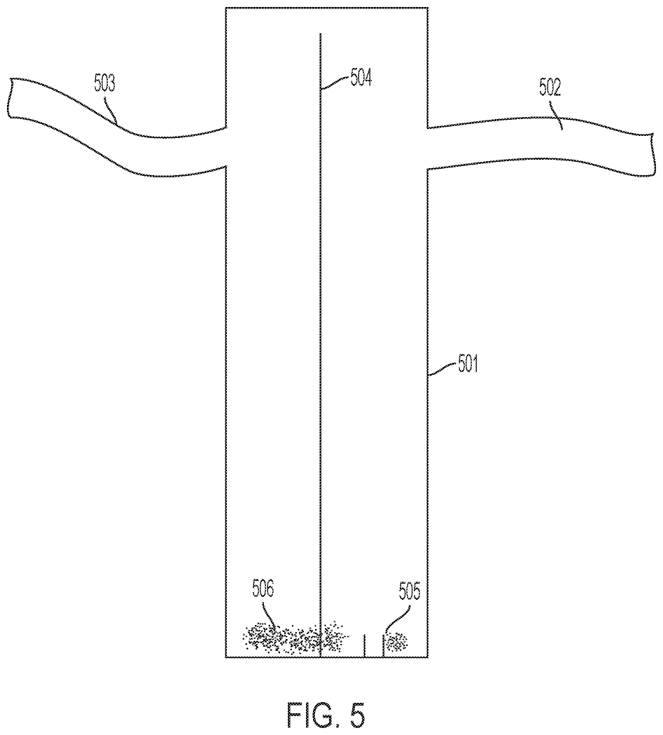

Turning to FIG. 5:

501 is a chamber used in the extraction of concentrated previously aerosolized matter.

502 is a gas input. In many use cases, the gas input will be oxygen.

503 is a gas input for gas plus vaporized matter. In many use cases, the gas input will be hydrogen.

504 is an optional catalyst to trigger combination of the gasses, normally oxygen and hydrogen combining to form water. In some cases the catalyst may be platinum.

505 is an optional spark generator.

506 represents previously vaporized matter that has become a concentrate.

In a different modality, the system may also be used to create concentrated versions of the aerosolized particulates. One method for doing this is to utilize a pure or substantially pure hydrogen atmosphere (although a non-pure hydrogen atmosphere may also be utilized, though with the problem of having remaining gas) in the heating chamber. In one aspect, the heating chamber temperature may be required to be less than a set amount and/or the heating elements turned off prior to any intermixing. In another aspect, the heating chamber is separate from (but normally operatively connected to the extraction chamber 501. As the hydrogen gas with aerosolized particles is released from the heating chamber, it is intermixed with oxygen in an approximate ratio of 2 hydrogen atoms for every oxygen atom (although it should be noted that some applications may utilize a different ratio). It should be noted that some additional gas, or an imperfect ratio slightly different than 2 to 1, may be used. To avoid having a vacuum within the chamber, it may be desirable to allow a replacement gas, in one aspect a non-oxygenated replacement gas, to flow into the chamber as the combination of the hydrogen and oxygen will remove those elements as gases (because they are converted to water).

The intermixed gas is then ignited, such as with a spark 505. Alternatively, a chemical reaction combining the hydrogen and oxygen is otherwise catalyzed, such as by utilizing platinum 504. As many of the suspended particles are hydrophobic or otherwise incapable of remaining in a water solution, the suspended particles are easily separated from the water. Such is the case with many cannabinoids. Rather than (or in addition to) separating the particles from the water, the water may also be evaporated, and the particles recovered from the surface the water was evaporated over. In one aspect, a wax paper or other substrate is placed in the oxygen/hydrogen combination chamber (either before the reaction or after) and the particles come out of the resulting water solution on the substrate. In this way, we can make a concentrate (such as a cannabis concentrate) without the use of dangerous solvents. Such a reaction will produce very little water. For example, approximately 620 liters of oxygen (uncompressed at room temperature) combines with approximately 1245 liters of hydrogen (uncompressed at room temperature) to produce 1 liter of water. As one gram of cannabis can be aerosolized in 1.245 liters of hydrogen (obviously this number is used for mathematical simplicity; one gram of cannabis can be aerosolized in less or more hydrogen gas), the water resulting from the reaction would be one milliliter. Indeed, because some moisture is normally expected of concentrates, it may be desirable to use higher amounts of hydrogen (by using a higher flow rate and/or a larger heating chamber) in order to generate additional moisture. Evaporation can be done but may not be necessary for many uses. Because high temperatures can eliminate many of the undesirables tastes and smells of plant matter, a concentrate generated in this manner is suitable not just for re-aerosolizing, but for consumption in food or liquids or by itself as well.

In another aspect, a very small filter may be utilized through which small atoms or molecules may pass (such as He or N₂). In this manner, the concentration of vaporized material in the gas (which can be any one of non-oxygenated gases, or gases combinations with oxygen at an insufficient level to support combustion) may be increased. Whether or not such a filter is used, the gas with suspended particles may be bubbled through a solution in which the particles of interest will be captured. For example, if capturing THC particles, an oil or alcohol solution may be utilized.

It should be understood that other substances can react to convert from a gas form to a liquid form. Such substances may also be used in place of oxygen and hydrogen. In addition, it should be understood that no additional substances may be required to accomplish the creation of a concentrate. Taking a nitrogen atmosphere as the heating atmosphere in this example, we use nitrogen as the gas into which 1 gram of cannabis is aerosolized. The gas is then cooled to at least −196 degrees C. At that point, the nitrogen becomes a liquid and the particulates may precipitate out of solution and be recovered in that manner.

In another aspect, it may be desirable to have a souvenir of use of the invention. As the primary (and in some cases, sole) material left behind after the process is carbon, we teach that some or all of the remaining carbon, whether cured of some impurities, all impurities, or no impurities, may be compressed into an industrial diamond.

Thus, an apparatus for creation of a cannabis concentrate may comprise, (i) a heating chamber containing cannabis to be concentrated, (ii) a heat source, (iii) a source of hydrogen gas, the source operably connected to the heating chamber and the heating chamber cleared of oxygen, the clearing being accomplished by replacing some or all of the oxygenated gas with hydrogen gas to below a LOC for substances being heated, including the hydrogen, wherein the cannabis is heated to a temperature above a boiling point of one or more cannabinoids, the hydrogen gas containing vaporized cannabis components are vented into a mixing chamber, oxygen in the form of O₂ is added to the mixing chamber; and a reaction is catalyzed in which the oxygen and the hydrogen gas combine to form water.

In some aspects, the catalyst may be combustion, platinum, or pressure, and in some embodiments a substance remaining after the oxygen and hydrogen have been substantially combined to form water is recovered.

In a related embodiment, one problem in cooking with certain oils is that the smoking point is too low for many applications. For example, coconut oil has a smoke point of approximately 177 degrees C., while MCT oil has a smoke point of 150 degrees C., rendering both a poor choice for deep frying. By contrast, oils typically associated with frying, have a higher smoking point, such as canola oil (smoking point ˜205 degrees C.) or peanut oil (smoking point ˜233 degrees C.). A related problem, also solved herein, is that of burning matter in an oven or microwave.

For persons with certain metabolic diseases, such as the CPT2 deficiency both inventors have, cooking with long chain fatty acids is strongly contraindicated. While MCT oil is the recommended fat for those with CPT2, it cannot even be used to brush vegetables for baking, as the smoking point is simply too low. Many “healthy fats” have a low smoking point.

The smoking point is the point at which oxidation of the fat begins. We teach utilizing a non-oxygenated atmosphere to cook, bake, or even deep fry with oils having a low smoking point.

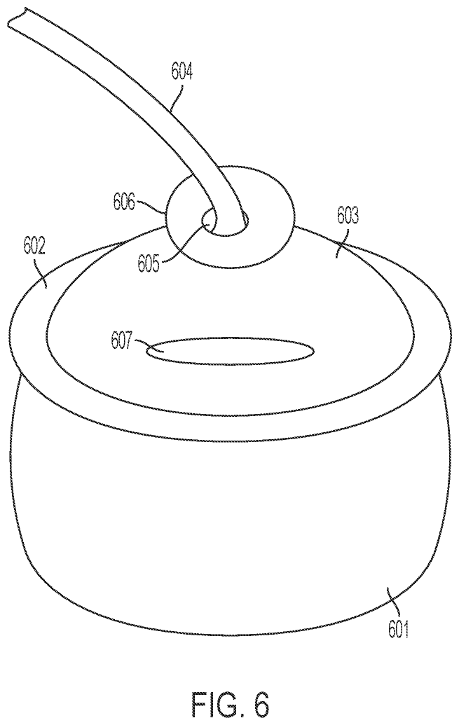

The first experiment showing the efficacy of this system is illustrated in FIG. 6. A pot 601 having a well fitting lid 603, the pot 601 or the lid 603 comprising a gasket, other material or surface 602 to substantially seal the lid 603 to the pot 601, with a hole 605 in the lid is used. A low smoking point oil (in this case, it was MCT oil) is placed in the pot 601 together with a substance to be cooked (in this case, potatoes). Piping 604 from a non-oxygenated gas source (in this case nitrogen) is placed in the hole 605 in the lid 603, preferably tightly fit to avoid leakage. The pot is placed on a heating surface and the non-oxygenated gas source is turned on. The food may be moved through the oil by shaking the pot. In an alternative embodiment, there may also be a large sealed passage 607 through the pot 601 or lid 603 for a stirring device. Alternatively, there may be a remotely controlled stirring device within the pot and/or lid (not shown).

A constant flow of non-oxygenated gas is one approach, but is not necessary in all instances. For example, a fully sealed container, such as a pressure cooker, may be voided or substantially voided of oxygen and sealed in that state. In another embodiment, the amount of oxygen present in the pot or heating chamber is below the LOC.

Thus, an apparatus for cooking with low smoke point oils may comprise (i) a heating chamber, (ii) a heat source, (iii) a source of non-oxygenated gas, the source operably connected to the heating chamber, wherein the heating chamber is capable of being cleared of oxygen to below a LOC for substances being heated by the heat source, such clearing accomplished by replacing atmospheric air with the source of non-oxygenated gas.

In some embodiments the heating chamber may be a pot that may be subjected to a constant flow of non-oxygenated gas, and the heating chamber may be sealed after being filled with non-oxygenated gas.

In another embodiment, a cooking device such as a microwave oven may be filled with non-oxygenated gas. This would allow very high temperatures to be reached without causing smoke. In one implementation, the contents of the microwave are allowed to cool before the door is allowed to open, thereby preventing a flash fire. Such a delay mechanism may be utilized in conjunction with the other methods for cooking in a non-oxygenated atmosphere. In a preferred implementation for a microwave application, the gas utilized is non-polar.

Whether a microwave, oven, or other cooking or heating device, replacement of the internal gas to below the LOC permits use without concern for accidental combustion or smoke.

The described systems and processes merely exemplify various embodiments of enhanced features. The present technology is not limited by these examples.

What is claimed is:

1. An apparatus for inducing sleep, comprising:
a vaporization chamber for placement of one or more sedating substances capable of becoming at least partially vaporized, the vaporization chamber sealed except for two or more conduits;
a first conduit of the two or more conduits operably coupled to a first source of a first gas that is comprised of Xenon;
a heating element capable of heating the vaporization chamber; and
a second conduit of the two or more conduits, the second conduit configured to transport vaporization gases and vaporized elements of the one or more sedating substances out of the vaporization chamber.

2. The apparatus of claim 1, wherein the one or more sedating substances comprises cannabis.

3. The apparatus of claim 1, wherein the vaporization chamber contains an amount of oxygen below a limiting oxygen concentration (LOC) for combustion of materials within the vaporization chamber.

4. The apparatus of claim 1, wherein the one or more sedating substances comprise a benzodiazepine.

5. An apparatus for rapidly administering a drug, comprising:
a chamber containing non-oxygenated gas and an amount of the drug;
the chamber operably coupled to a heat source;
applying heat from the heat source upon actuation of the apparatus until a boiling temperature of the drug is reached or exceeded;
the apparatus providing an indication that sufficient drug has been vaporized;
the apparatus ceasing heating when the indication is provided; and
a tube or other conduit to allow inhalation of the gas and vaporized drug from the apparatus.

6. The apparatus of claim 5, where the drug is a blood thinner.

7. The apparatus of claim 5, where the drug is salicylic acid.

8. The apparatus of claim 5, where the drug is an erectile dysfunction drug.

9. The apparatus of claim 5, where the drug is naloxone.

10. The apparatus of claim 5, where the drug is a benzodiazepine.

11. The apparatus of claim 5, where the apparatus is operably connected to a communication modality and upon actuation of the apparatus, the apparatus actuating the communication modality and requesting assistance.

12. An apparatus for vaporizing plant matter, comprising:
a cartridge-receiving fitting;
a cartridge containing plant matter for vaporization;
a heating element internal or external to the cartridge;
the cartridge filled with a gas having an amount of oxygen less than a LOC for combustion of the plant materials;
a gas-bearing connector capable of connecting to the cartridge-receiving fitting, thereby allowing the gas to be inhaled.

13. The apparatus of claim 12, further comprising a resistance heating element contained within the cartridge and a power source for the resistance heating element.

14. The apparatus of claim 12, further comprising a penetrating device to puncture one or more surfaces of the cartridge.

15. The apparatus of claim 12, further comprising a conduit for transport of contents of the cartridge for inhalation.

16. The apparatus of claim 15, further comprising a penetrating device to puncture a portion of the cartridge operably connected to the conduit.

17. An apparatus for vaporizing plant matter, comprising:
a vaporization chamber;
a pump for removal of an amount of gas from the chamber;
the apparatus calibrated to remove enough gas to lower the boiling point of one or more substances within the chamber to a temperature at or below a set temperature;
the apparatus, upon actuation, lowering a pressure to match the temperature that the vaporization chamber is at or will be heated to;
the apparatus equalizing the pressure after boiling to substantially match ambient atmospheric pressure; and
a gas-bearing connector capable of connecting to the vaporization chamber and allowing the gas to be inhaled.

18. The apparatus of claim 17, further comprising a heating element capable of raising the temperature within the vaporization chamber.

19. The apparatus of claim 17, wherein the apparatus contains a computer programmed to calculate an optimum combination of temperature and pressure for vaporization of a given substance, and to actuate pressure reduction or heating elements.

20. The apparatus of claim 17, where the gas remaining in the chamber is non-oxygenated.

21. The apparatus of claim 17, where the gas remaining in the chamber is below a LOC for the pressure within the chamber.

22. An apparatus for cooking with low smoke point oils, comprising:
a heating chamber;
a heat source;
a source of non-oxygenated gas, the source operably connected to the heating chamber;
the heating chamber capable of being cleared of oxygen to below a LOC for substances being heated by the heat source, such clearing accomplished by replacing atmospheric air with the source of non-oxygenated gas.

23. The apparatus of claim 22, wherein the heating chamber is a pot.

24. The apparatus of claim 22, wherein the heating chamber is subjected to a constant flow of non-oxygenated gas.

25. The apparatus of claim 22, wherein the heating chamber is sealed after being filled with non-oxygenated gas.

26. An apparatus for creation of cannabis concentrate, comprising:
a heating chamber containing cannabis;
a heat source;
a source of hydrogen gas, the source operably connected to the heating chamber;
the heating chamber cleared of oxygen, the clearing being accomplished by replacing some or all of the oxygenated gas with hydrogen gas to below a LOC for substances being heated, including the hydrogen;
heating the cannabis to above a boiling point of one or more cannabinoids;
venting the hydrogen gas containing vaporized cannabis components into a mixing chamber;
adding oxygen in the form of O₂ to the mixing chamber; and
catalyzing a reaction in which the oxygen and the hydrogen gas combine to form water.

27. The apparatus of claim 26, wherein the catalyst is combustion.

28. The apparatus of claim 26, wherein the catalyst is platinum.

29. The apparatus of claim 26, wherein a substance remaining after the oxygen and hydrogen have been substantially combined to form water is recovered.

30. The apparatus of claim 26, where the catalyst is pressure.
